(19) United States
(12) Patent Application Publication
(10) Pub. No.: US 2024/0228494 A1
(43) Pub. Date: Jul. 11, 2024

(54) COMPOUNDS

(71) Applicant: PATHIOS THERAPEUTICS LIMITED, ABINGDON (GB)

(72) Inventors: David MILLER, ABINGDON, UK (GB); Tom MCCARTHY, ABINGDON, UK (GB); Alan NAYLOR, ABINGDON, UK (GB); Gavin MILNE, ABINGDON, UK (GB)

(21) Appl. No.: 18/556,260

(22) PCT Filed: Apr. 25, 2022
(86) PCT No.: PCT/GB22/51042

§ 371 Date: Oct. 19, 2023

(30) Foreign Application Priority Data
Apr. 26, 2021 (GB) 2105942.3
Dec. 2, 2021 (GB) 2117433.9

(51) Int. Cl. C07D 471/18; A61K 31/4353; A61K 31/519

(52) U.S. Cl. CPC C07D 471/18; A61K 31/4353; A61K 31/519

(57) ABSTRACT

One aspect of the invention relates to a compound of formula (I), or a pharmaceutically acceptable salt or solvate thereof, (I) ring A is an is optionally substituted 5 or 6 membered aromatic or heteroaromatic ring; Y is selected from CH₂, C═N—OH, and CR₁₀R_{10′}; Rₐ and R_{b} are each independently selected from H and alkyl; Z is selected from O, S, NR₁₇ and CR₁₈; X is selected from O and NH; p is 0, 1 or 2; q is 0 or 1; and r is 0 or 1; wherein at least one of p, q and r is other than zero, and with the proviso that: (i) when r is 0, p is 1 and q is 1, when X is NH, Z is other than O; (ii) when r and q are both 0, and p is 1, Z is not O; R₁, R₄, and R₅ are each independently selected from H, alkyl, alkoxy, OH, F, Cl, Br, and I; R₂ and R₃ are each independently selected from H, F, Cl, Br, I, CN, methoxy, haloalkyl, haloalkoxy and CO₂-alkyl; R₁₀ and R_{10′} are each independently selected from H, F, alkyl, and haloalkyl; R₁₅ and R₁₆ are each independently selected from H, alkoxy, alkyl and OH; R₁₇ is selected from H, CN, OH, alkoxy and alkyl; R₁₈ is NO₂ or CN; and wherein the compound is other than: (6S,9R)-10-benzyl-4-chloro-6,7,8,9-tetrahydro-5H-6,9-epiminocyclohepta[d]-pyrimidine; (6S,9R)-10-benzyl-1,5,6,7,8,9-hexahydro-4H-6,9-epiminocyclohepta[d]pyrimidin-4-one; (6R,9S)-10-benzyl-1,5,6,7,8,9-hexahydro-4H-6,9-epiminocyclohepta[d]pyrimidin-4-one; 10-benzyl-1,5,6,7,8,9-hexahydro-4H-6,9-epiminocyclohepta[d]pyrimidin-4-one; 10-(2,4,6-trimethoxybenzyl)-6,7,8,9-tetrahydro-5H-5,8-epiminocyclohepta[d]-pyrimidine; and 2-methoxy-5-((6,7,8,9-tetrahydro-5H-5,8-epiminocyclohepta[d]pyrimidin-10-yl)methyl)-benzonitrile. Further aspects of the invention relate to compounds of formula (I) for use in the field of immuno-oncology, immunology, and related applications.

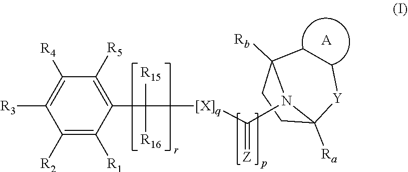

The present invention relates to compounds that are capable of modulating GPR65. The compounds have potential therapeutic applications in the treatment of a variety of disorders including proliferative and immune disorders.

### BACKGROUND TO THE INVENTION

GPR65 is a Gs-coupled G protein-coupled receptor (GPCR) that is primarily expressed in immune cells and is activated by acidic extracellular pH to cause increases in cytoplasmic cyclic adenosine monophosphate (cAMP) (Wang, 2004). It has long been known that tumours typically undergo a switch in cellular metabolism from oxidative phosphorylation to aerobic glycolysis, which in turn results in an acidic extracellular microenvironment (Damaghi, 2013). Recently, it has been shown that this acidic microenvironment causes GPR65 activation in tumour-associated macrophages, resulting in an increase in cytoplasmic cAMP leading to transcription of the inducible cAMP early repressor (ICER). This, in turn, suppresses the secretion of tumour necrosis factor alpha (TNFα) to bias the macrophages toward an anti-inflammatory, tumour-permissive phenotype (Bohn, 2018). This GPR65-dependent pathway therefore appears to represent a mechanism by which tumours exploit their acidic microenvironment to evade detection by the immune system.

Autoimmune diseases are also often associated with an acidic local microenvironment (for instance, an inflamed joint). Recent studies also suggest that GPR65 acts through ICER in CD4+ T cells, to suppress IL-2 and hence bias cells toward an inflammatory Th17 phenotype, which is associated with increased pathogenicity in the context of autoimmune disease (Korn, 2009). Supporting this is the recent finding that ICER is required for Th17 differentiation (Yoshida, 2016) as well as that agonism of GPR65 leads to an increase in Th17 differentiation (Hernandez, 2018). Indeed, mutations in the GPR65 locus are associated with several autoimmune diseases, such as multiple sclerosis, ankylosing spondylitis, inflammatory bowel disease, and Crohn's disease (Gaublomme, 2015). One recent study found that mice with CD4+ T cells lacking GPR65 were protected from developing the disease autoimmune encephalomyelitis (EAE) (Gaublomme, 2015).

Thus, GPR65 appears to act through ICER to promote an anti-inflammatory and tumour-permissive phenotype in tumour associated macrophages and an inflammatory Th17 phenotype in CD4+ T cells that is associated with autoimmune disease. GPR65 signalling, therefore, represents an attractive pathway for therapeutic intervention for the treatment of both cancer and autoimmune diseases. There is therefore an ongoing need to develop new small molecule GPR65 modulators.

The present invention seeks to provide compounds that are capable of modulating GPR65. As made clear from the above discussion, such compounds have potential therapeutic applications in the treatment of a variety of disorders, including proliferative disorders and immune disorders, as well as asthma and chronic obstructive pulmonary disease.

### STATEMENT OF INVENTION

A first aspect of the invention relates to a compound of formula (I), or a pharmaceutically acceptable salt or solvate thereof,

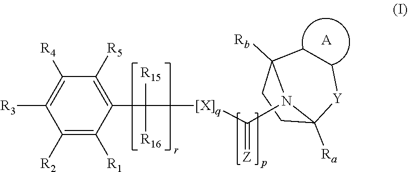

wherein:
ring A is a 5 or 6 membered aromatic or heteroaromatic ring, wherein said aromatic or heteroaromatic ring is optionally substituted with one or more substituents selected from halo, CN, alkoxy, NR₁₁R_{11′}, OH, CONR₁₉R₂₀, SO₂NR₁₉R₂₀, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R_{11′}, OH, alkyl, haloalkyl, and aralkyl;
Y is selected from CH₂, C═N—OH, and CR₁₀R_{10′};
Rₐ and R_{b} are each independently selected from H and alkyl;
Z is selected from O, S, NR₁₇ and CR₁₈;
X is selected from O and NH;
p is 0, 1 or 2;
q is 0 or 1; and
r is 0 or 1;
wherein at least one of p, q and r is other than zero, and with the proviso that:
(i) when r is 0, p is 1 and q is 1, when X is NH, Z is other than 0;
(ii) when r and q are both 0, and p is 1, Z is not O;
R₁, R₄, and R₅ are each independently selected from H, alkyl, alkoxy, OH, F, Cl, Br, and I;
R₂ and R₃ are each independently selected from H, F, Cl, Br, I, CN, methoxy, haloalkyl, haloalkoxy and CO₂-alkyl;
R₁₀ and R_{10′} are each independently selected from H, F, alkyl, and haloalkyl;
R₁₁ and R_{11′} are each independently selected from H, alkyl, haloalkyl, COR₁₂, CO₂R₁₂ and SO₂R₁₃;
R₁₂ and R₁₃ are each independently alkyl;
R₁₅ and R₁₆ are each independently selected from H, alkoxy, alkyl and OH;
R₁₇ is selected from H, CN, OH, alkoxy and alkyl;
R₁₈ is NO₂ or CN;
each R₁₉ is independently H or alkyl; and
each R₂₀ is independently alkyl;
   and wherein the compound is other than:
(6S,9R)-10-benzyl-4-chloro-6,7,8,9-tetrahydro-5H-6,9-epiminocyclohepta[d]-pyrimidine;
(6S,9R)-10-benzyl-1,5,6,7,8,9-hexahydro-4H-6,9-epiminocyclohepta[d]pyrimidin-4-one;
(6R,9S)-10-benzyl-1,5,6,7,8,9-hexahydro-4H-6,9-epiminocyclohepta[d]pyrimidin-4-one;
10-benzyl-1,5,6,7,8,9-hexahydro-4H-6,9-epiminocyclohepta[d]pyrimidin-4-one;
10-(2,4,6-trimethoxybenzyl)-6,7,8,9-tetrahydro-5H-5,8-epiminocyclohepta[d]-pyrimidine; and
2-methoxy-5-((6,7,8,9-tetrahydro-5H-5,8-epiminocyclohepta[d]pyrimidin-10-yl)methyl)-benzonitrile.

Advantageously, the presently claimed compounds are capable of modulating GPR65, thereby rendering the compounds of therapeutic interest in the treatment of various disorders, for example, in the field of oncology, immuno-oncology, and immunology.

Another aspect of the invention relates to a compound of formula (Ii), or a pharmaceutically acceptable salt or solvate thereof,

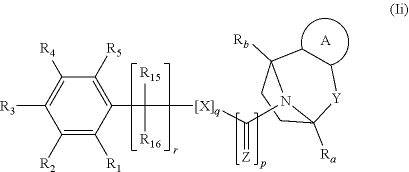

wherein:
ring A is a 5 or 6 membered aromatic or heteroaromatic ring, wherein said aromatic or heteroaromatic ring is optionally substituted with one or more substituents selected from halo, CN, alkoxy, NR₁₁R_{11′}, OH, CONR₁₉R₂₀, SO₂NR₁₉R₂₀, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R_{11′}, OH, alkyl, haloalkyl, and aralkyl;
Y is selected from CH₂, C═N—OH, and CR₁₀R_{10′};
Rₐ and R_{b} are each independently selected from H and alkyl;
Z is selected from O, S, NR₁₇ and CR₁₈;
X is selected from O and NH;
p is 0
q is 0; and
r is 1;
R₁, R₄, and R₅ are each independently selected from H, alkyl, alkoxy, OH, F, C, Br, and I;
R₂ is selected from H, F, Cl, Br, I, CN, methoxy, haloalkyl, haloalkoxy and CO₂-alkyl;
R₃ is selected from H, F, Cl, Br, I, CN, haloalkyl, haloalkoxy and CO₂-alkyl
with the proviso that at least one of R₁-R₅ must be other than H;
R₁₀ and R_{10′} are each independently selected from H, F, alkyl, and haloalkyl;
R₁₁ and R₁₁ are each independently selected from H, alkyl, haloalkyl, COR₁₂, CO₂R₁₂ and SO₂R₁₃;
R₁₂ and R₁₃ are each independently alkyl;
R₁₅ and R₁₆ are each H;
R₁₇ is selected from H, CN, OH, alkoxy and alkyl;
R₁₈ is NO₂ or CN;
each R₁₉ is independently H or alkyl; and
each R₂₀ is independently alkyl.

Another aspect of the invention relates to a compound of formula (Ih), or a pharmaceutically acceptable salt or solvate thereof,

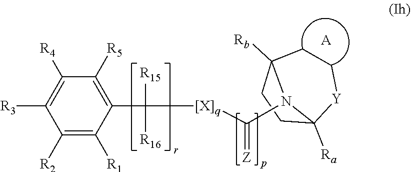

wherein:
ring A is a 5 or 6 membered aromatic or heteroaromatic ring, wherein said aromatic or heteroaromatic ring is optionally substituted with one or more substituents selected from halo, CN, alkoxy, NR₁₁R_{11′}, OH, CONR₁₉R₂₀, SO₂NR₁₉R₂₀, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R_{11′}, OH, alkyl, haloalkyl, and aralkyl;
Y is selected from CH₂, C═N—OH, and CR₁₀R_{10′};
Rₐ and R_{b} are each independently selected from H and alkyl;
Z is selected from O, S, NR₁₇ and CR₁₃;
X is selected from O and NH;
p is 0, 1 or 2;
q is 0 or 1; and
r is 0 or 1;
wherein at least one of p, q and r is other than zero, and with the proviso that:
(i) when r is 0, p is 1 and q is 1, when X is NH, Z is other than O;
(ii) when r and q are both 0, and p is 1, Z is not O;
R₁, R₄, and R₅ are each independently selected from H, alkyl, alkoxy, OH, F, C, Br, and I;
R₂ and R₃ are each independently selected from H, F, C, Br, I, CN, methoxy, haloalkyl, haloalkoxy and CO₂-alkyl;
R₁₀ and R_{10′} are each independently selected from H, F, alkyl, and haloalkyl;
R₁₁ and R_{11′} are each independently selected from H, alkyl, haloalkyl, COR₁₂, CO₂R₁₂ and SO₂R₁₃;
R₁₂ and R₁₃ are each independently alkyl;
R₁₅ and R₁₆ are each independently selected from H, alkoxy, alkyl and OH;
R₁₇ is selected from H, CN, OH, alkoxy and alkyl;
R₁₈ is NO₂ or CN;
each R₁₉ is independently H or alkyl; and
each R₂₀ is independently alkyl;
for use as a medicament.

Another aspect of the invention relates to a compound as described above for use in treating or preventing a disorder selected from a proliferative disorder, an immune disorder, asthma, chronic obstructive pulmonary disease (COPD) and acute respiratory distress syndrome (ARDS).

Another aspect of the invention relates to a pharmaceutical composition comprising a compound as described above and a pharmaceutically acceptable diluent, excipient, or carrier.

Another aspect of the invention relates to a pharmaceutical composition as described above for use as a medicament.

Another aspect of the invention relates to a pharmaceutical composition as described above for use in treating or preventing a disorder selected from a proliferative disorder, an immune disorder, asthma, chronic obstructive pulmonary disease (COPD) and acute respiratory distress syndrome (ARDS).

Another aspect of the invention relates to a method of treating a disorder, comprising administering to a subject a compound or a pharmaceutical composition as described above.

Another aspect of the invention relates to a compound as defined herein, or a pharmaceutically acceptable salt or solvate thereof, for use in treating or preventing a GPR65-associated disease or disorder.

Another aspect of the invention relates to the use of a compound as defined herein, or a pharmaceutically acceptable salt or solvate thereof, in the preparation of a medicament for treating or preventing a GPR65-associated disease or disorder in a subject.

Another aspect of the invention relates to the use of a compound as defined herein, or a pharmaceutically acceptable salt or solvate thereof, in the preparation of a medicament for treating or preventing a disorder selected from a proliferative disorder, an immune disorder, asthma, chronic obstructive pulmonary disease (COPD) and acute respiratory distress syndrome (ARDS).

### DETAILED DESCRIPTION

The present invention relates to compounds that are capable of modulating GPR65.

“Alkyl” is defined herein as a straight-chain or branched alkyl radical, preferably C₁₋₂₀ alkyl, more preferably C₁₋₁₂ alkyl, even more preferably C₁₋₁₀ alkyl or C₁₋₆ alkyl, for example, methyl, ethyl, propyl, isopropyl, butyl, isobutyl, tert-butyl, pentyl, hexyl. More preferably, the alkyl is a C₁₋₃ alkyl.

As used herein, the term “aryl” or “aromatic” refers to a C₆₁₂ aromatic group, which may be benzocondensed, for example, phenyl or naphthyl.

“Haloalkyl” is defined herein as a straight-chain or branched alkyl radical as defined above, for example, methyl, ethyl, propyl, isopropyl, butyl, isobutyl, tert-butyl, pentyl, hexyl, that is substituted with one or more halogen atoms (that may be the same or different), such as fluorine, chlorine, bromine, and iodine. Preferred examples are CF₃ and CHF₂, with CF₃ being particularly preferred.

“Alkoxy” is defined herein as an oxygen atom bonded to an alkyl group as defined above. Preferably the alkoxy group is a C₁₋₂₀ alkoxy, more preferably C₁₋₁₂ alkoxy, even more preferably C₁₋₁₀ alkoxy or C₁₋₆ alkoxy, for example methoxy, ethoxy, propoxy, isopropoxy, butoxy, isobutoxy, tert-butoxy, pentoxy and hexoxy. A particularly preferred example is methoxy (—OCH₃).

“Heteroaryl” or “heteroaromatic” is defined herein as a monocyclic or bicyclic C₂₋₁₂ aromatic ring comprising one or more heteroatoms (that may be the same or different), such as oxygen, nitrogen or sulphur. Examples of suitable heteroaryl groups include thienyl, furanyl, pyrrolyl, pyridinyl, oxazolyl, thiazolyl, imidazolyl, pyrazolyl, isoxazolyl, isothiazolyl, oxadiazolyl, triazolyl, thiadiazolyl etc. and benzo derivatives thereof, such as benzofuranyl, benzothienyl, benzimidazolyl, indolyl, isoindolyl, indazolyl etc.; or pyridyl, pyrazinyl, pyrimidinyl, pyridazinyl, triazinyl etc. and benzo derivatives thereof, such as quinolinyl, isoquinolinyl, cinnolinyl, phthalazinyl, quinazolinyl, quinoxalinyl, naphthyridinyl etc.

“Aralkyl” is defined herein as an alkyl group as defined above substituted by one or more aryl groups as defined above.

As used herein, preferably alkyl is C₁-C₆ alkyl, haloalkyl is C₁-C₆ haloalkyl, and alkoxy is C₁-C₆ alkoxy.

### Structural Representation of the Compounds

The compounds of the invention comprise a structure wherein ring A is an optionally substituted 5- or 6-membered aromatic or heteroaromatic ring fused to a bicyclic nitrogen-containing moiety to form a tricyclic structure. The resulting tricyclic structure can exist in two different configurations as depicted below (bridge substituents Rₐ and R_{b} are omitted in the following representations for clarity):

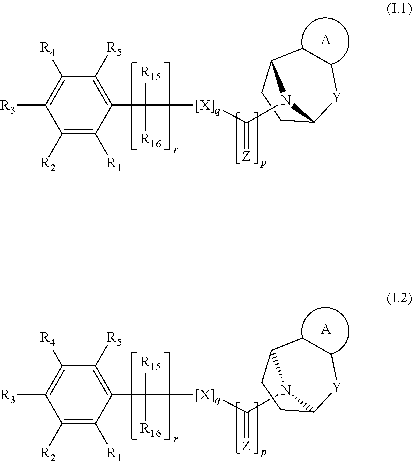

For the avoidance of doubt, the invention encompasses the compounds in either of the above configurations, as well as mixtures thereof, including racemic mixtures.

The compounds described herein contain an optionally substituted 5 or 6-membered aromatic or heteroaromatic ring A, which is fused to the bicyclic nitrogen-containing moiety to form a tricyclic structure. The optional substituents are selected from halo, CN, alkoxy, NR₁₁R_{11′}, OH, CONR₁₉R₂₀, SO₂NR₁₉R₂₀, alkyl, haloalkyl, aralkyl, aryl and heteroaryl. In some instances, ring A can exist in more than one tautomeric form. By way of illustration, where the heteroaromatic ring is a 6 membered ring substituted by an OH group, ring A can exist as two possible tautomers as shown below:

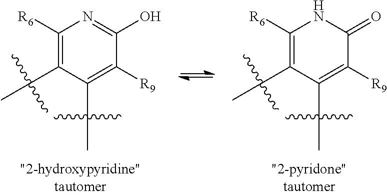

The 2-pyridone tautomer is believed to be the predominant solid state form. In solution, the energy difference between the two tautomeric forms is understood to be very small and is dependent on the polarity of the solvent. The skilled person would appreciate that other hydroxy substituted N-containing heteroaromatic groups (e.g. pyrimidine, other pyridine regioisomers) can be similarly represented in tautomeric form as shown above. The term “heteroaromatic” as used herein encompasses all tautomeric forms of the compounds.

Preferably, ring A is as defined herein, where the wavy lines denote attachment to the ring containing N and Y:

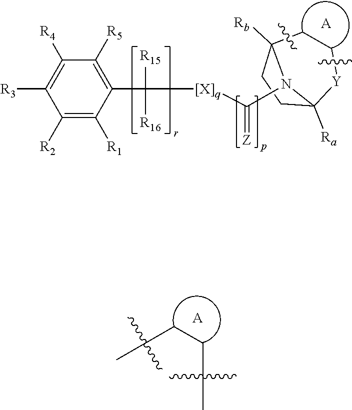

### Compounds of Formula (I), (I′) and (Ii)

One aspect of the invention relates to a compound of formula (I′), or a pharmaceutically acceptable salt or solvate thereof:

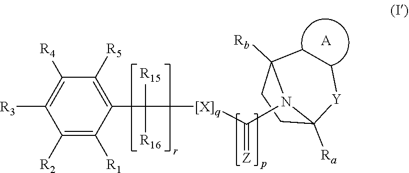

wherein:
ring A is a 5 or 6 membered aromatic or heteroaromatic ring, wherein said aromatic or heteroaromatic ring is optionally substituted with one or more substituents selected from halo, CN, alkoxy, NR₁₁R_{11′}, OH, CONR₁₉R₂₀, SO₂NR₁₉R₂₀, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R_{11′}, OH, alkyl, haloalkyl, and aralkyl;
Y is selected from CH₂, C═N—OH, and CR₁₀R_{10′};
Rₐ and R_{b} are each independently selected from H and alkyl;
Z is selected from O, S, NR₁₇ and CR₁₈;
X is selected from O and NH;
p is 0, 1 or 2;
q is 0 or 1; and
r is 0 or 1;
wherein at least one of p, q and r is other than zero, and with the proviso that when r is 0, p is 1 and q is 1, when X is NH, Z is other than O;
R₁, R₄, and R₅ are each independently selected from H, alkyl, alkoxy, OH, F, Cl, Br, and I;
R₂ and R₃ are each independently selected from H, F, Cl, Br, I, CN, methoxy, haloalkyl, haloalkoxy and CO₂-alkyl;
R₁₀ and R_{10′} are each independently selected from H, F, alkyl, and haloalkyl;
R₁₁ and R_{11′} are each independently selected from H, alkyl, haloalkyl, COR₁₂, CO₂R₁₂ and SO₂R₁₃;
R₁₂ and R₁₃ are each independently alkyl;
R₁₅ and R₁₆ are each independently selected from H, alkoxy, alkyl and OH;
R₁₇ is selected from H, CN, OH, alkoxy and alkyl;
R₁₈ is NO₂ or CN;
each R₁₉ is independently H or alkyl; and
each R₂₀ is independently alkyl.

Preferably, the compound is other than:

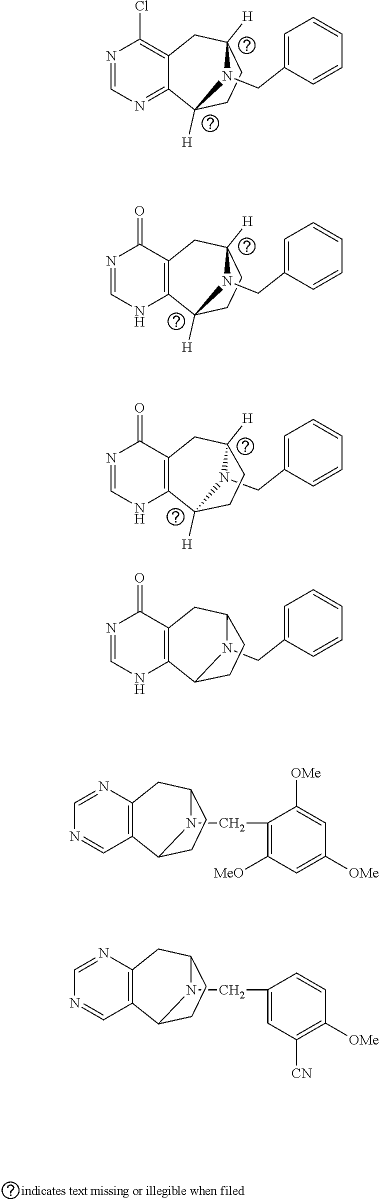 [SMILES: Clc1ncnc2c1C[C@@H]1CC[C@H]2N1Cc1ccccc1 ; O=c1nc[nH]c2c1C[C@@H]1CC[C@H]2N1Cc1ccccc1 ; O=c1nc[nH]c2c1C[C@H]1CC[C@@H]2N1Cc1ccccc1 ; O=c1nc[nH]c2c1CC1CCC2N1Cc1ccccc1 ; COc1cc(OC)c(CN2C3CCC2c2cncnc2C3)c(OC)c1 ; COc1ccc(CN2C3CCC2c2cncnc2C3)cc1C#N]

One aspect of the invention relates to a compound of formula (I), or a pharmaceutically acceptable salt or solvate thereof,

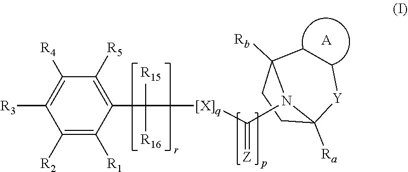

wherein:
ring A is a 5 or 6 membered aromatic or heteroaromatic ring, wherein said aromatic or heteroaromatic ring is optionally substituted with one or more substituents selected from halo, CN, alkoxy, NR₁₁R_{11′}, OH, CONR₁₉R₂₀, SO₂NR₁₉R₂₀, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R_{11′}, OH, alkyl, haloalkyl, and aralkyl;
Y is selected from CH₂, C═N—OH, and CR₁₀R_{10′};
Rₐ and R_{b} are each independently selected from H and alkyl;
Z is selected from O, S, NR₁₇ and CR₁₈;
X is selected from O and NH;
p is 0, 1 or 2;
q is 0 or 1; and
r is 0 or 1;
wherein at least one of p, q and r is other than zero, and with the proviso that:
(i) when r is 0, p is 1 and q is 1, when X is NH, Z is other than 0;
(ii) when r and q are both 0, and p is 1, Z is not O;
R₁, R₄, and R₅ are each independently selected from H, alkyl, alkoxy, OH, F, Cl, Br, and I;
R₂ and R₃ are each independently selected from H, F, Cl, Br, I, CN, alkoxy, haloalkyl, haloalkoxy and CO₂-alkyl;
R₁₀ and R_{10′} are each independently selected from H, F, alkyl, and haloalkyl;
R₁₁ and R_{11′} are each independently selected from H, alkyl, haloalkyl, COR₁₂, CO₂R₁₂ and SO₂R₁₃;
R₁₂ and R₁₃ are each independently alkyl;
R₁₁ and R_{11′} are each independently selected from H, alkoxy, alkyl and OH;
R₁₇ is selected from H, CN, OH, alkoxy and alkyl;
R₁₈ is NO₂ or CN;
each R₁₉ is independently H or alkyl; and
each R₂₀ is independently alkyl;
   and wherein the compound is other than:
(6S,9R)-10-benzyl-4-chloro-6,7,8,9-tetrahydro-5H-6,9-epiminocyclohepta[d]-pyrimidine;
(6S,9R)-10-benzyl-1,5,6,7,8,9-hexahydro-4H-6,9-epiminocyclohepta[d]pyrimidin-4-one;
(6R,9S)-10-benzyl-1,5,6,7,8,9-hexahydro-4H-6,9-epiminocyclohepta[d]pyrimidin-4-one;
10-benzyl-1,5,6,7,8,9-hexahydro-4H-6,9-epiminocyclohepta[d]pyrimidin-4-one;
10-(2,4,6-trimethoxybenzyl)-6,7,8,9-tetrahydro-5H-5,8-epiminocyclohepta[d]-pyrimidine; and
2-methoxy-5-((6,7,8,9-tetrahydro-5H-5,8-epiminocyclohepta[d]pyrimidin-10-yl)methyl)-benzonitrile.

In one preferred embodiment, Z is selected from O, S and NR₁₇.

In one preferred embodiment, p is 1, q is 1 and r is 0.

In one preferred embodiment, p is 1, q is 1 and r is 0, X is NH, and Z is selected from S and NR₁₇, i.e. the tricyclic group is linked to the aryl group by a —C(═S)—NH— or —C(═NR₁₇)—NH— group.

In one preferred embodiment, p is 1, q is 1 and r is 0, X is NH, and Z is S, i.e. the tricyclic group is linked to the aryl group by a —C(═S)—NH— group.

Thus, in one particularly preferred embodiment, the compound of the invention is of formula (Ia):

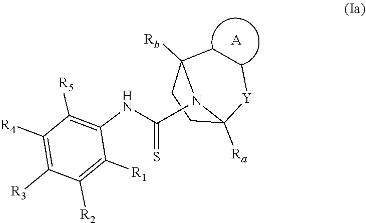

or a pharmaceutically acceptable salt or solvate thereof, where R₁₋₅, Rₐ, R_{b}, Y and A are as defined above.

In one preferred embodiment, p is 1, q is 1 and r is 0, X is NH, and Z is NR₁₇, i.e. the tricyclic group is linked to the aryl group by a —C(═NR₁₇)—NH— group.

Thus, in one particularly preferred embodiment, the compound of the invention is of formula (Ib):

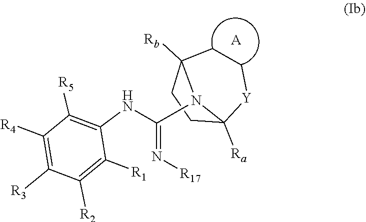

or a pharmaceutically acceptable salt or solvate thereof, where R₁₋₅, Rₐ, R_{b}, R₁₇, Y and A are as defined above.

More preferably, R₁₇ is selected from H, CN, OH, Me and MeO, and is even more preferably CN.

In one preferred embodiment, p is 1, q is 1 and r is 0, X is 0 and Z is O, i.e. the tricyclic group is linked to the aryl group by a —C(═O)—O— group.

Thus, in one particularly preferred embodiment, the compound of the invention is of formula (Ic):

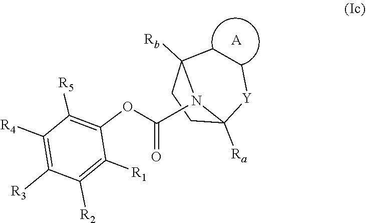

or a pharmaceutically acceptable salt or solvate thereof, where R₁₋₅, Rₐ, R_{b}, Y and A are as defined above.

In one preferred embodiment, p is 1, q is 1 and r is 0, X is O or NH, and Z is CR₁₈, preferably where R₁₈ is NO₂, i.e. the tricyclic group is linked to the aryl group by a —C(═CR₁₈)—NH— or —C(═CR₁₈)—O— group.

In one preferred embodiment, p is 1, q is 1 and r is 0, X is NH, and Z is CR₁₈.

Thus, in one particularly preferred embodiment, the compound of the invention is of formula (Id):

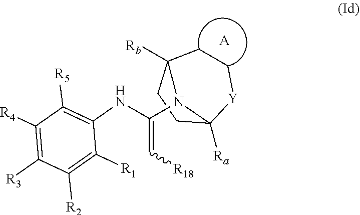

or a pharmaceutically acceptable salt or solvate thereof, where R₁₋₅, Rₐ, R_{b}, R₁₈, Y and A are as defined above.

In one preferred embodiment, p is 1, q is 1 and r is 0, X is NH, Z is CR₁₈, and R₁₈ is NO₂.

In another preferred embodiment, p is 1, q is 1 and r is 0, X is NH, Z is CR₁₈, and R₁₈ is CN.

In another preferred embodiment, p is 1, q is 0 and r is 1.

In one preferred embodiment, p is 1, q is 0 and r is 1, Z is O, and R₁₅ and R₁₆ are each independently selected from H, OMe, Me and OH, i.e. the tricyclic group is linked to the aryl group by a —C(═O)—CR₁₅R₁₆— group.

Thus, in one particularly preferred embodiment, the compound of the invention is of formula (Ie):

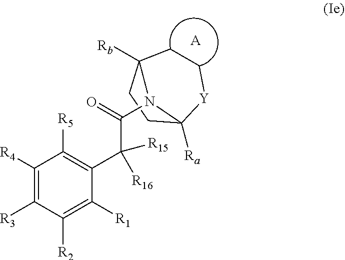

or a pharmaceutically acceptable salt or solvate thereof, where R₁₋₅, Rₐ, R_{b}, R₁₅, R₁₆, Y and A are as defined above.

In a more preferred embodiment, p is 1, q is 0 and r is 1, Z is O and R₁₅ and R₁₆ are both H, or one of R₁₅ and R₁₆ is H and the other is selected from H and OH.

In another preferred embodiment, p is 0, q is 0 and r is 1, i.e. the tricyclic group is linked to the aryl group by a —CR₁₅R₁₆— group.

Thus, in one particularly preferred embodiment, the compound of the invention is of formula (If):

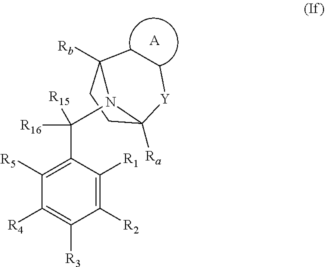

or a pharmaceutically acceptable salt or solvate thereof, where R₁₋₅, R₁₅, R₁₆, Rₐ, R_{b}, Y and A are as defined above.

Preferably, for this embodiment, R₁₅ and R₁₆ are both H. In another preferred embodiment, at least one of R₁₅ and R₁₆ is other than H. Preferably, for compounds of formula (If), at least one of R₁-R₅ is other than H. More preferably, for compounds of formula (If), R₁, R₄, and R₅ are each independently selected from H, alkyl, alkoxy, OH, F, Cl, Br, and I; R₂ is selected from H, F, Cl, Br, I, CN, methoxy, haloalkyl, haloalkoxy and CO₂-alkyl; R₃ is selected from H, F, Cl, Br, I, CN, haloalkyl, haloalkoxy and CO₂-alkyl, with the proviso that at least one of R₁-R₅ must be other than H.

In another preferred embodiment, p is 2, q is 0 and r is 0. Preferably, for this embodiment, Z is O, i.e. the tricyclic group is linked to the aryl group by a —C(═O)—C(═O)— group.

Thus, in one particularly preferred embodiment, the compound of the invention is of formula (Ig):

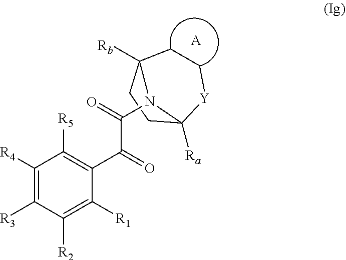

or a pharmaceutically acceptable salt or solvate thereof, where R₁₋₅, Rₐ, R_{b}, Y and A are as defined above.

Preferably, when p and q are both 0, r is not 1.

Preferably, when p and q are both 0, and r is 1, R₁₅ and R₁₆ are not both H.

Preferably, when r and q are both 0, p is not 1 when Z is O.

The following definitions and preferred embodiments apply to each of subformulae (I) and (Ia)-(Ii) described herein.

In one preferred embodiment of the invention, ring A is a benzene, pyridine, pyridone, pyridine N-oxide, pyrimidine, pyrimidone, pyridazine, pyrazine, or isoxazole ring that is optionally substituted with one or more substituents selected from F, Cl, Br, I, CN, C₁-C₆ alkoxy, NR₁₁R_{11′}, CONR₁₉R₂₀, SO₂NR₁₉R₂₀, OH, C₁-C₆ alkyl, phenyl, and C₁-C₆ haloalkyl.

In one preferred embodiment, ring A is selected from a pyridine moiety and a pyrimidine moiety, each of which may be optionally substituted. Particularly preferred substituents are as defined above for ring A, and more preferably, include halo and OH.

In one particularly preferred embodiment, the compound of the invention is of formula (I) as defined above, with the proviso that:
when r is 0, p is 1 and q is 1, when X is NH, Z is other than 0;
when r is 1, p is 0, and q is 0, R₁₅ and R₁₆ are not both H; and
when r is 0, p is 1, and q is 0, Z is other than 0.

In one preferred embodiment, ring A is selected from the groups (i)-(xix):

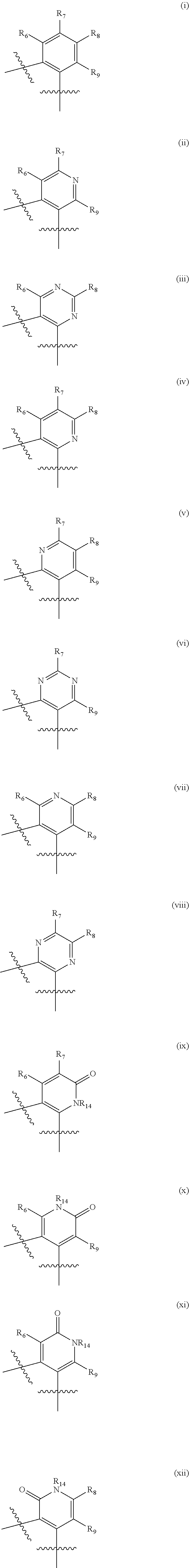

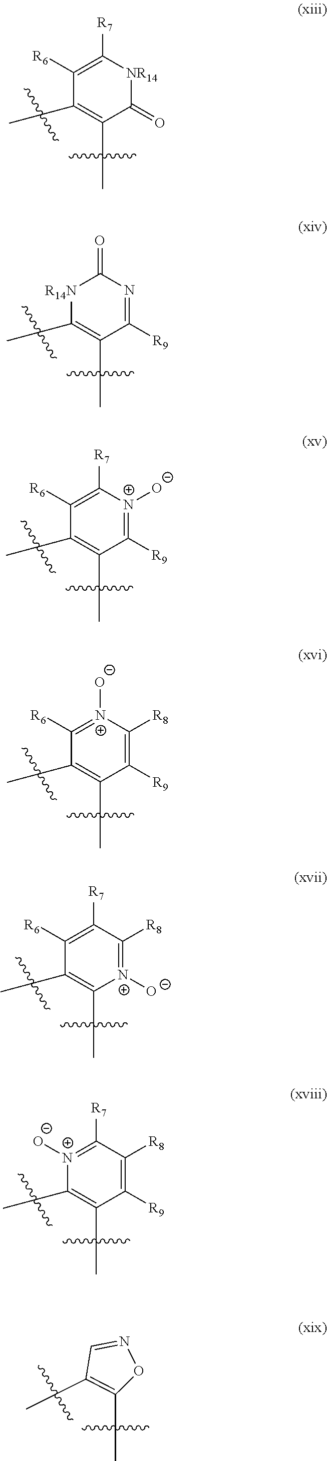

wherein R₆, R₇, R₃, and R₉ are each independently selected from H, F, Cl, Br, I, CN, alkoxy, NR₁₁R_{11′}, OH, alkyl, phenyl, CONR₁₉R₂₀, SO₂NR₁₉R₂₀ and haloalkyl, and R₁₄ is H or alkyl, more preferably H.

In one preferred embodiment, R₆, R₇, R₃, and R₉ are each independently selected from H, F, Cl, Br, CN, OMe, NR₁₁R_{11′} and OH.

In one preferred embodiment, R₆, R₇, R₃, and R₉ are each independently selected from H, F, Cl, Br, CN, OMe, NH₂, NHBu, NHCO₂Bu and OH. More preferably, R₆, R₇, R₃, and R₉ are each independently selected from H and F.

In one preferred embodiment, R₁₄ is H or methyl. More preferably, R₁₄ is H.

In one preferred embodiment, R₁₀ and R_{10′} are each independently selected from H, F, Me, and CF₃.

In one preferred embodiment, R₁₀ and R_{10′} are each independently selected from H, Me, CF₃, COR₁₂, CO₂R₁₂ and SO₂R₁₃, preferably wherein R₁₂ and R₁₃ are each Me.

In one preferred embodiment each R₁₉ is independently C₁₋₆alkyl, more preferably Me.

In one preferred embodiment each R₂₀ is independently C₁₋₆alkyl, more preferably Me.

In one preferred embodiment, ring A is selected from (ii), (iii) and (x):

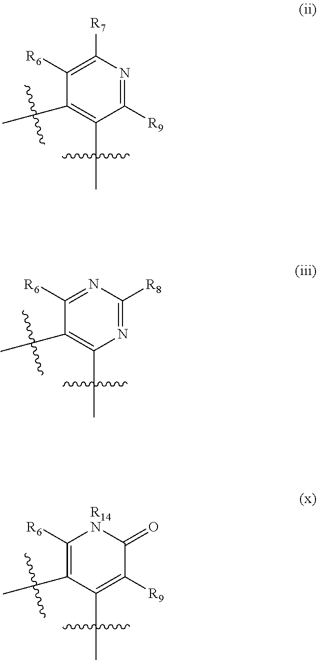

In one preferred embodiment, ring A is of group (ii). Preferably, where A is of group (ii), R₆, R₇ and R₉ are each independently selected from H and F. More preferably, R₉ is F and R₆ and R₇ are both H.

In one preferred embodiment, ring A is of group (iii). Preferably, where A is of group (iii), R₆ and R₈ are each independently selected from H and F, and are more preferably both H.

In one preferred embodiment, ring A is of group (x). Preferably, where A is of group (x), R₆, R₉ and R₁₄ are all H.

In one preferred embodiment, Rₐ and R_{b} are each independently selected from H and methyl.

In one preferred embodiment, one of Rₐ and R_{b} is alkyl (more preferably methyl) and the other is H.

In one particularly preferred embodiment, Rₐ and R_{b} are both H.

In one preferred embodiment, Y is selected from CH₂ and C═N—OH, and is more preferably CH₂.

In one preferred embodiment, Y is selected from CH₂ and CR₁₀R_{10′}, and is more preferably CH₂.

In one preferred embodiment, at least one of R₁, R₂, R₃, R₄ and R₅ is other than H.

In one preferred embodiment, R₂ and R₃ are each independently selected from F, Cl, Br, I, CN, CO₂-alkyl, C₁-C₆ haloalkyl and C₁-C₆ haloalkoxy.

In one preferred embodiment, R₂ and R₃ are each independently selected from F, C, Br, I, CN, and C₁-C₆ haloalkyl.

In one preferred embodiment, R₂ and R₃ are each independently selected from C, Br, and CF₃, and more preferably independently selected from C and CF₃.

In one preferred embodiment, R₂ and R₃ are both Cl, or one of R₂ and R₃ is CF₃ and the other is selected from Cl and Br.

In one preferred embodiment, one of R₂ and R₃ is Cl and the other is OCF₃ or OCHF₂.

In one preferred embodiment, one of R₂ and R₃ is Cl and the other is CO₂Me.

In one preferred embodiment, R₂ and R₃ are both Cl, or one of R₂ and R₃ is Cl and the other is CF₃.

In one preferred embodiment, R₂ and R₃ are both Cl.

In one preferred embodiment, one of R₂ and R₃ is CF₃ and the other is Cl.

In one preferred embodiment, one of R₂ and R₃ is CF₃ and the other is Br.

In one preferred embodiment, R₁, R₄, and R₅ are each independently selected from H, Me, OMe, OH, F, Cl, Br, and I.

In one preferred embodiment, R₁ is H.

In one preferred embodiment, R₄ is H.

In one preferred embodiment, R₂ is selected from Br and Cl.

In one preferred embodiment, R₃ is selected from CF₃ and Cl.

In one preferred embodiment, R₁ and R₄ are both H.

In one preferred embodiment, R₅ is selected from H, F and CN, and is preferably H or F.

In one preferred embodiment:
R₁ is H;
R₂ is selected from Br and Cl;
R₃ is selected from CF₃ and Cl;
R₄ is H; and
R₅ is selected from H and F.

In one preferred embodiment:
A is selected from a group (ii), (iii) and (x) as defined above;
Y is CH₂;
Rₐ and R_{b} are H;
R₁ is H;
R₂ is selected from Br and Cl;
R₃ is selected from CF₃ and Cl;
R₄ is H; and
Rₛ is selected from H and F.

Another aspect of the invention relates to a compound of formula (Ii), or a pharmaceutically acceptable salt or solvate thereof,

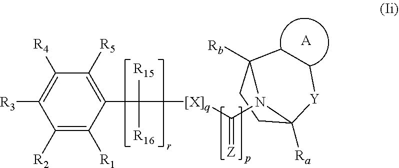

wherein:
ring A is a 5 or 6 membered aromatic or heteroaromatic ring, wherein said aromatic or heteroaromatic ring is optionally substituted with one or more substituents selected from halo, CN, alkoxy, NR₁₁R_{11′}, OH, CONR₁₉R₂₀, SO₂NR₁₉R₂₀, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R_{11′} OH, alkyl, haloalkyl, and aralkyl;
Y is selected from CH₂, C═N—OH, and CR₁₀R_{10′};
Rₐ and R_{b} are each independently selected from H and alkyl;
Z is selected from O, S, NR₁₇ and CR₁₈;
X is selected from O and NH;
p is 0
q is 0; and
r is 1;
R₁, R₄, and R₅ are each independently selected from H, alkyl, alkoxy, OH, F, Cl, Br, and I;
R₂ is selected from H, F, Cl, Br, I, CN, methoxy, haloalkyl, haloalkoxy and CO₂-alkyl;
R₃ is selected from H, F, Cl, Br, I, CN, haloalkyl, haloalkoxy and CO₂-alkyl
with the proviso that at least one of R₁-R₅ must be other than H;
R₁₀ and R_{10′} are each independently selected from H, F, alkyl, and haloalkyl;
R₁₁ and R_{11′} are each independently selected from H, alkyl, haloalkyl, COR₁₂, CO₂R₁₂ and SO₂R₁₃;
R₁₂ and R₁₃ are each independently alkyl;
R₁₅ and R₁₆ are each H;
R₁₇ is selected from H, CN, OH, alkoxy and alkyl;
R₁₈ is NO₂ or CN;
each R₁₉ is independently H or alkyl; and
each R₂₀ is independently alkyl.

Thus, the compound of the invention is of formula:

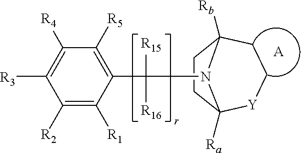

or a pharmaceutically acceptable salt or solvate thereof,
wherein:
Y, A, Rₐ and R_{b} are as defined above;
r is 1;
R₁, R₄, and R₅ are each independently selected from H, alkyl, alkoxy, OH, F, Cl, Br, and I;
R₂ is selected from H, F, Cl, Br, I, CN, methoxy, haloalkyl, haloalkoxy and CO₂-alkyl;
R₃ is selected from H, F, Cl, Br, I, CN, haloalkyl, haloalkoxy and CO₂-alkyl
with the proviso that at least one of R₁-R₅ must be other than H; and
R₁₅ and R₁₆ are each H.

Preferred definitions for A, Y, Rₐ, R_{b} and R₁-R₅ are as set out above for compounds of formula (I).

In one particularly preferred embodiment, the compound is of formula (I.1):

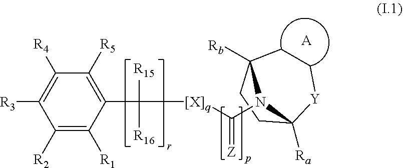

wherein ring A, and groups X, Y, Z, p, q, r, Rₐ, R_{b} and R₁-R₅ are as described in any of the above embodiments.

In one preferred embodiment, the compound is in enantiomerically pure form. In one preferred embodiment, the compound is in the form of a mixture that is enantiomerically enriched with a compound of formula (I.1).

In another embodiment, the compound is of formula (I.2):

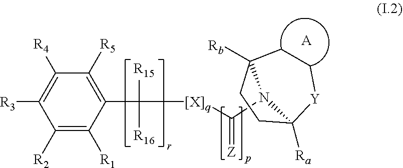

wherein ring A, and groups X, Y, Z, p, q, r, Rₐ, R_{b} and R₁-R₅ are as described in any of the above embodiments.

In one preferred embodiment, the compound is in enantiomerically pure form. In one preferred embodiment, the compound is in the form of a mixture that is enantiomerically enriched with a compound of formula (I.2).

In one preferred embodiment, the compound is in the form of a mixture comprising a compound of formula (I.1) and its corresponding enantiomer of formula (I.2). In one preferred embodiment, the mixture is a racemic mixture, i.e. a 50:50 mixture of a compound of formula (I.1) and its corresponding enantiomer of formula (I.2).

Racemic mixtures can be used to prepare enantiomerically pure compounds of formula (I.1) or (I.2) by separating the compounds of formula (I.1) or (I.2) by standard methods, for example by chemical resolution using optically active acid or by the use of column chromatography or reverse-phase column chromatography using a substantially optically active (or “chiral”) stationary phase as known to those skilled in the art. Racemic mixtures can also be used to prepare enantiomerically enriched mixtures of compounds of formula (I.1) or (I.2). Mixtures enriched with either a compound of formula (I.1) or (I.2) can also be obtained from the appropriate enantiomerically enriched precursors.

In one preferred embodiment of the invention, the compound is in the form of a mixture comprising enantiomers wherein the weight:weight ratio is at least approximately 2:1 or greater, preferably at least approximately 5:1 or greater, most preferably at least approximately 10:1 or greater in favour of the enantiomer that displays significant in vitro and/or in vivo activity (the eutomer).

In one particularly preferred embodiment, the compound is in the form of a mixture comprising a compound of formula (I.1) and its corresponding enantiomer of formula (I.2), wherein the weight:weight ratio of said compound of formula (I.1) to said compound of formula (I.2) is greater than 1.05:1, more preferably, greater than 2:1, even more preferably greater than 5:1, even more preferably greater than 10:1.

In one particularly preferred embodiment, the compound is in the form of a mixture comprising a compound of formula (I.1) and its corresponding enantiomer of formula (I.2), which is substantially enriched with said compound of formula (I.1).

In one embodiment, the compound is in the form of a mixture comprising a compound of formula (I.1) and its corresponding enantiomer of formula (I.2), wherein the weight:weight ratio of said compound of formula (I.2) to said compound of formula (I.1) is greater than 1.05:1, more preferably, greater than 2:1, even more preferably greater than 5:1, even more preferably greater than 10:1.

In one embodiment, the compound is in the form of a mixture comprising a compound of formula (I.1) and its corresponding enantiomer of formula (I.2), which is substantially enriched with said compound of formula (I.2).

In one preferred embodiment, the compound is selected from the following:

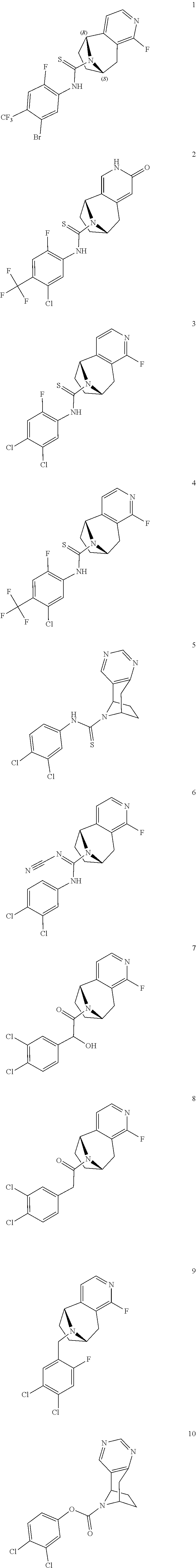

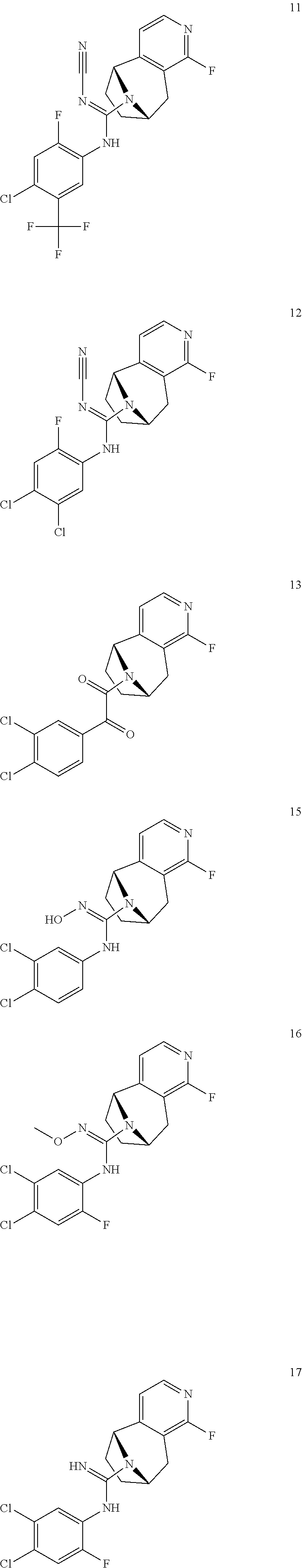 [SMILES: N#C/N=C(/Nc1cc(C(F)(F)F)c(Cl)cc1F)N1[C@H]2CC[C@@H]1c1ccnc(F)c1C2 ; N#C/N=C(/Nc1cc(Cl)c(Cl)cc1F)N1[C@H]2CC[C@@H]1c1ccnc(F)c1C2 ; O=C(C(=O)N1[C@H]2CC[C@@H]1c1ccnc(F)c1C2)c1ccc(Cl)c(Cl)c1 ; O/N=C(\Nc1ccc(Cl)c(Cl)c1)N1[C@H]2CC[C@@H]1c1ccnc(F)c1C2 ; CO/N=C(\Nc1cc(Cl)c(Cl)cc1F)N1[C@H]2CC[C@@H]1c1ccnc(F)c1C2 ; N=C(Nc1cc(Cl)c(Cl)cc1F)N1[C@H]2CC[C@@H]1c1ccnc(F)c1C2]

and enantiomers thereof, and mixtures of enantiomers thereof, including racemic mixtures, and pharmaceutically acceptable salts and solvates thereof.

In one preferred embodiment, the compound of formula (I) is selected from the following compounds as shown herein: 1-6, 8-16, and enantiomers thereof, and mixtures of enantiomers thereof, including racemic mixtures, and pharmaceutically acceptable salts and solvates thereof.

In one preferred embodiment, the compound of formula (I) is selected from the following compounds as shown herein: 1-6, 11, 12, 15 and 16, and enantiomers thereof, and mixtures of enantiomers thereof, including racemic mixtures, and pharmaceutically acceptable salts and solvates thereof.

In another preferred embodiment, the compound of formula (I) is selected from the following:
(5R,8S)—N-(5-bromo-2-fluoro-4-(trifluoromethyl)phenyl)-1-fluoro-6,7,8,9-tetrahydro-5H-5,8-epiminocyclohepta[c]pyridine-10-carbothioamide;
(5S,8R)—N-(5-bromo-2-fluoro-4-(trifluoromethyl)phenyl)-1-fluoro-6,7,8,9-tetrahydro-5H-5,8-epiminocyclohepta[c]pyridine-10-carbothioamide;
(6S,9R)—N-(5-chloro-2-fluoro-4-(trifluoromethyl)phenyl)-3-oxo-3,5,6,7,8,9-hexahydro-2H-6,9-epiminocyclohepta[c]pyridine-10-carbothioamide;
(6R,SR)—N-(5-chloro-2-fluoro-4-(trifluoromethyl)phenyl)-3-oxo-3,5,6,7,8,9-hexahydro-2H-6,9-epiminocyclohepta[c]pyridine-10-carbothioamide;
(5R,8S)—N-(4,5-dichloro-2-fluorophenyl)-1-fluoro-6,7,8,9-tetrahydro-5H-5,8-epiminocyclo-hepta[c]pyridine-10-carbothioamide;
(5S,8R)—N-(4,5-dichloro-2-fluorophenyl)-1-fluoro-6,7,8,9-tetrahydro-5H-5,8-epiminocyclo-hepta[c]pyridine-10-carbothioamide;
(5R,8S)—N-(5-chloro-2-fluoro-4-(trifluoromethyl)phenyl)-1-fluoro-6,7,8,9-tetrahydro-5H-5,8-epiminocyclohepta[c]pyridine-10-carbothioamide;
(5S,8R)—N-(5-chloro-2-fluoro-4-(trifluoromethyl)phenyl)-1-fluoro-6,7,8,9-tetrahydro-5H-5,8-epiminocyclohepta[c]pyridine-10-carbothioamide;
(5R,8S)—N-(3,4-dichlorophenyl)-6,7,8,9-tetrahydro-5H-5,8-epiminocyclohepta[d]pyrimidine-10-carbothioamide;
(5S,8R)—N-(3,4-dichlorophenyl)-6,7,8,9-tetrahydro-5H-5,8-epiminocyclohepta[d]pyrimidine-10-carbothioamide;
(5R,8S)—N′-cyano-N-(3,4-dichlorophenyl)-1-fluoro-6,7,8,9-tetrahydro-5H-5,8-epiminocyclo-hepta[c]pyridine-10-carboximidamide;
(5S,8R)—N′-cyano-N-(3,4-dichlorophenyl)-1-fluoro-6,7,8,9-tetrahydro-5H-5,8-epiminocyclo-hepta[c]pyridine-10-carboximidamide;
2-(3,4-dichlorophenyl)-1-((5R,8S)-1-fluoro-6,7,8,9-tetrahydro-5H-5,8-epiminocyclohepta-[c]pyridin-10-yl)-2-hydroxyethan-1-one;
2-(3,4-dichlorophenyl)-1-((5S,8R)-1-fluoro-6,7,8,9-tetrahydro-5H-5,8-epiminocyclohepta-[c]pyridin-10-yl)-2-hydroxyethan-1-one;
2-(3,4-dichlorophenyl)-1-((5R,8S)-1-fluoro-6,7,8,9-tetrahydro-5H-5,8-epiminocyclohepta-[c]pyridin-10-yl)ethan-1-one;
2-(3,4-dichlorophenyl)-1-((5S,8R)-1-fluoro-6,7,8,9-tetrahydro-5H-5,8-epiminocyclohepta-[c]pyridin-10-yl)ethan-1-one;
(5R,8S)-10-(4,5-dichloro-2-fluorobenzyl)-1-fluoro-6,7,8,9-tetrahydro-5H-5,8-epiminocyclo-hepta[c]pyridine;
(5S,8R)-10-(4,5-dichloro-2-fluorobenzyl)-1-fluoro-6,7,8,9-tetrahydro-5H-5,8-epiminocyclo-hepta[c]pyridine;
3,4-dichlorophenyl (5R,8S)-6,7,8,9-tetrahydro-5H-5,8-epiminocyclohepta[d]pyrimidine-10-carboxylate;
3,4-dichlorophenyl (5S,8R)-6,7,8,9-tetrahydro-5H-5,8-epiminocyclohepta[d]pyrimidine-10-carboxylate
(5R,8S)—N-(4-Chloro-2-fluoro-5-(trifluoromethyl)phenyl)-N′-cyano-1-fluoro-6,7,8,9-tetrahydro-5H-5,8-epiminocyclohepta[c]pyridine-10-carboximidamide
(5S,8R)—N-(4-Chloro-2-fluoro-5-(trifluoromethyl)phenyl)-N′-cyano-1-fluoro-6,7,8,9-tetrahydro-5H-5,8-epiminocyclohepta[c]pyridine-10-carboximidamide
(5R,8S)—N′-cyano-N-(4,5-dichloro-2-fluorophenyl)-1-fluoro-6,7,8,9-tetrahydro-5H-5,8-epiminocyclohepta[c]pyridine-10-carboximidamide
(5S,8R)—N′-cyano-N-(4,5-dichloro-2-fluorophenyl)-1-fluoro-6,7,8,9-tetrahydro-5H-5,8-epiminocyclohepta[c]pyridine-10-carboximidamide
1-(3,4-Dichlorophenyl)-2-((5R,8S)-1-fluoro-6,7,8,9-tetrahydro-5H-5,8-epiminocyclohepta-[c]pyridin-10-yl)ethane-1,2-dione
1-(3,4-Dichlorophenyl)-2-((5S,8R)-1-fluoro-6,7,8,9-tetrahydro-5H-5,8-epiminocyclohepta-[c]pyridin-10-yl)ethane-1,2-dione
(5R,8S)—N-(4,5-dichloro-2-fluorophenyl)-1-fluoro-N′-hydroxy-6,7,8,9-tetrahydro-5H-5,8-epiminocyclohepta[c]pyridine-10-carboximidamide
(5S,8R)—N-(4,5-dichloro-2-fluorophenyl)-1-fluoro-N′-hydroxy-6,7,8,9-tetrahydro-5H-5,8-epiminocyclohepta[c]pyridine-10-carboximidamide
(5R,8S)—N-(4,5-dichloro-2-fluorophenyl)-1-fluoro-N′-methoxy-6,7,8,9-tetrahydro-5H-5,8-epiminocyclohepta[c]pyridine-10-carboximidamide
(5S,8R)—N-(4,5-dichloro-2-fluorophenyl)-1-fluoro-N′-methoxy-6,7,8,9-tetrahydro-5H-5,8-epiminocyclohepta[c]pyridine-10-carboximidamide
(5R,8S)—N′-(4,5-dichloro-2-fluorophenyl)-1-fluoro-6,7,8,9-tetrahydro-5H-5,8-epiminocyclo-hepta[c]pyridine-10-carboximidamide
(5S,8R)—N′-(4,5-dichloro-2-fluorophenyl)-1-fluoro-6,7,8,9-tetrahydro-5H-5,8-epiminocyclo-hepta[c]pyridine-10-carboximidamide;
(±)-N-(5-bromo-2-fluoro-4-(trifluoromethyl)phenyl)-1-fluoro-6,7,8,9-tetrahydro-5H-5,8-epiminocyclohepta[c]pyridine-10-carbothioamide;
(±)-N-(5-chloro-2-fluoro-4-(trifluoromethyl)phenyl)-3-oxo-3,5,6,7,8,9-hexahydro-2H-6,9-epiminocyclohepta[c]pyridine-10-carbothioamide;
(±)-N-(4,5-dichloro-2-fluorophenyl)-1-fluoro-6,7,8,9-tetrahydro-5H-5,8-epiminocyclo-hepta[c]pyridine-10-carbothioamide;
(±)-N-(5-chloro-2-fluoro-4-(trifluoromethyl)phenyl)-1-fluoro-6,7,8,9-tetrahydro-5H-5,8-epiminocyclohepta[c]pyridine-10-carbothioamide;
(±)-N-(3,4-dichlorophenyl)-6,7,8,9-tetrahydro-5H-5,8-epiminocyclohepta[d]pyrimidine-10-carbothioamide;
(±)-N′-cyano-N-(3,4-dichlorophenyl)-1-fluoro-6,7,8,9-tetrahydro-5H-5,8-epiminocyclo-hepta[c]pyridine-10-carboximidamide;
2-(3,4-dichlorophenyl)-1-(±)-(1-fluoro-6,7,8,9-tetrahydro-5H-5,8-epiminocyclohepta-[c]pyridin-10-yl)-2-hydroxyethan-1-one;
2-(3,4-dichlorophenyl)-1-(±)-(1-fluoro-6,7,8,9-tetrahydro-5H-5,8-epiminocyclohepta-[c]pyridin-10-yl)ethan-1-one;
(±)-10-(4,5-dichloro-2-fluorobenzyl)-1-fluoro-6,7,8,9-tetrahydro-5H-5,8-epiminocyclo-hepta[c]pyridine;
3,4-dichlorophenyl-(±)-6,7,8,9-tetrahydro-5H-5,8-epiminocyclohepta[d]pyrimidine-10-carboxylate (±)-N-(4-chloro-2-fluoro-5-(trifluoromethyl)phenyl)-N′-cyano-1-fluoro-6,7,8,9-tetrahydro-5H-5,8-epiminocyclohepta[c]pyridine-10-carboximidamide (±)-N′-cyano-N-(4,5-dichloro-2-fluorophenyl)-1-fluoro-6,7,8,9-tetrahydro-5H-5,8-epimino-cyclohepta[c]pyridine-10-carboximidamide
1-(3,4-Dichlorophenyl)-2-(±)-(1-fluoro-6,7,8,9-tetrahydro-5H-5,8-epiminocyclohepta-[c]pyridin-10-yl)ethane-1,2-dione
(±)-N-(4,5-dichloro-2-fluorophenyl)-1-fluoro-N′-hydroxy-6,7,8,9-tetrahydro-5H-5,8-epimino-cyclohepta[c]pyridine-10-carboximidamide (±)-N-(4,5-dichloro-2-fluorophenyl)-1-fluoro-N′-methoxy-6,7,8,9-tetrahydro-5H-5,8-epimino-cyclohepta[c]pyridine-10-carboximidamide; and
(±)-N′-(4,5-dichloro-2-fluorophenyl)-1-fluoro-6,7,8,9-tetrahydro-5H-5,8-epiminocyclohepta-[c]pyridine-10-carboximidamide.
and pharmaceutically acceptable salts and solvates thereof.

### Process

A further aspect of the invention relates to a process for preparing a compound as defined herein wherein p is 1, q is 1, r is 0, X is NH, and Z is S, said process comprising treating a compound of formula (III), where R₁-R₅ are as defined above, with a solution of silver trifluoromethanethiolate and KBr in a suitable solvent to form a compound of formula (IV). Preferably, the solvent is MeCN. The compound of formula (IV) is then reacted with a compound of formula (II), wherein A, Y, Rₐ and R_{b} are as defined herein, in a suitable solvent (preferably DCM), in the presence of a base (preferably DIPEA):

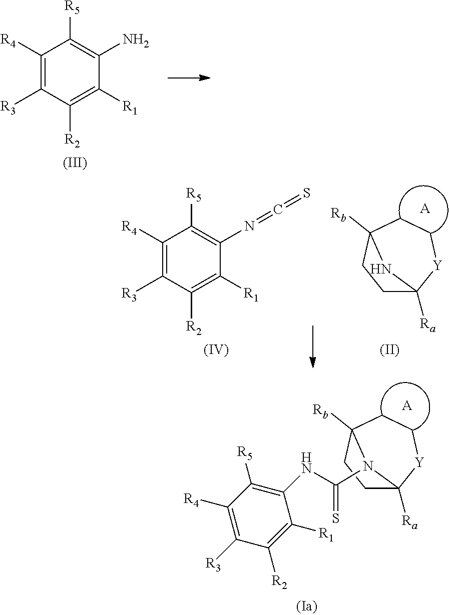

A further aspect of the invention relates to a process for preparing a compound as defined herein wherein p is 1, q is 1, r is 0, X is NH, and Z is S, said process comprising treating a compound of formula (III) above, where R₁-R₅ are as defined above, with a solution of elemental sulfur, potassium fluoride and trimethyl(trifluoromethyl)silane in a suitable solvent to form a compound of formula (IV). The compound of formula (IV) is then reacted with a compound of formula (II), wherein A, Y, Rₐ and R_{b} are as defined above, in a suitable solvent. Preferably, the reaction takes place in an organic solvent. Suitable organic solvents include, but are not limited to, dichloromethane, tetrahydrofuran and dimethylformamide, or mixtures of two or more thereof. The skilled person would understand that other solvents would also be suitable.

A further aspect of the invention relates to a process for preparing a compound as defined herein wherein p is 1, q is 1, r is 0, X is NH, Z is NR₁₇ and R₁₇ is CN, said process comprising treating a compound of formula (IV), where R₁-R₅ are as defined above, with a solution of sodium hydrogencyanamide in ethanol to form a compound of formula (V). The compound of formula (V) is then treated with a compound of formula (II) in a suitable solvent, such as DMF, for example. The skilled person would understand that other solvents would also be suitable. Preferably, the reaction between the compound of formula (II) and the compound of formula (V) takes place in the presence of a carboxyl activating agent, more preferably a carbodiimide such as 1-ethyl-3-(3-dimethylaminopropyl)carbodiimide, e.g. EDCl·HCl.

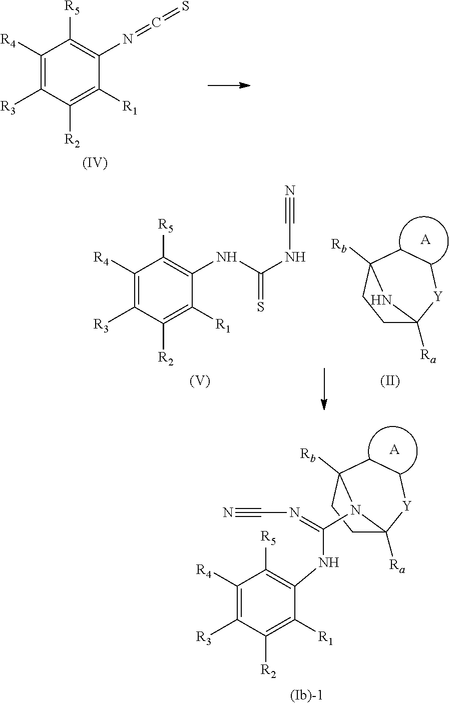

A further aspect of the invention relates to a process for preparing a compound as defined herein wherein p is 1, q is 0, r is 1 and Z is O, said process comprising treating a compound of formula (VI), where R₁-R₅, R₁₅ and R₁₆ are as defined herein, with a compound of formula (II) in a suitable solvent.

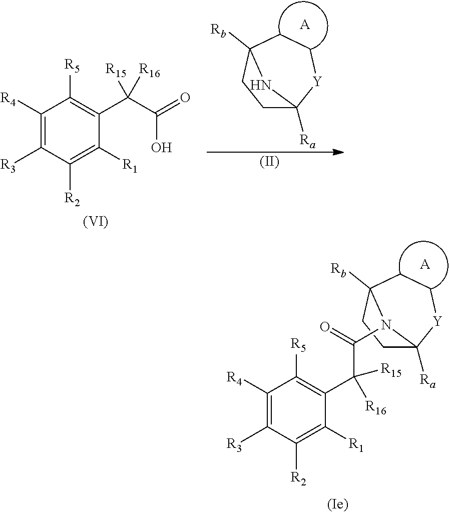

Preferably the reaction takes place in the presence of an activating agent, for example, (1-[bis(dimethylamino)methylene]-1H-1,2,3-triazolo[4,5-b]pyridinium 3-oxide hexafluoro-phosphate, (hexafluorophosphate azabenzotriazole tetramethyl uronium; HATU) which generates an active ester from a carboxylic acid. Preferably, the reaction takes place in the presence of a base, for example, N,N-diisopropylethylamine (DIPEA), or triethylamine. Typically DMF is used as solvent, although other polar aprotic solvents can also be used. Compounds in which one of R₁₅ and R₁₆ is OH can be subsequently oxidised by treating with a suitable oxidising agent (for example 2,2,6,6-tetramethylpiperidinyloxy (2,2,6,6-tetramethylpiperidine 1-oxyl or “TEMPO”) and calcium hypochlorite, or Dess-Martin periodinane; see Reddy, Sabbasani Rajasekhara; et al Synthetic Communications (2012), 42(23), 3493-3503; Kitagawa, Osamu; et al Journal of Organic Chemistry (2003), 68(25), 9851-9853).

A further aspect of the invention relates to a process for preparing a compound as defined herein wherein p is 0, q is 0, r is 1, and R₁₅ and R₁₆ are both H, said process comprising treating a compound of formula (VII), where R₁-R₅ are as defined above, with a compound of formula (II) in the presence of a reducing agent in a suitable solvent.

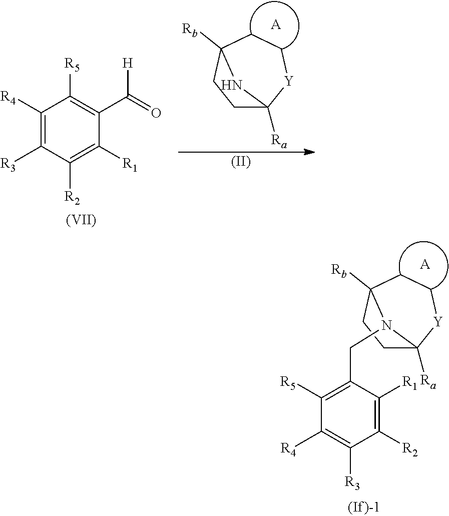

Preferably, the reducing agent is sodium triacetoxyborohydride (STAB) although the skilled person would understand that other reducing agents would also be suitable. Preferably, the solvent is dichoromethane, although the skilled person would understand that other organic solvents would also be suitable.

A further aspect of the invention relates to a process for preparing a compound as defined herein wherein p is 1, q is 1, r is 0, X is O and Z is O, said process comprising treating a compound of formula (VIII), where R₁-R₅ are as defined above, with trisphosgene in the presence of a suitable solvent, and adding thereto a base (preferably triethylamine). The resulting mixture is then reacted with a compound of formula (II) in a suitable solvent. Preferably, the solvent is dichoromethane, although the skilled person would understand that other organic solvents would also be suitable.

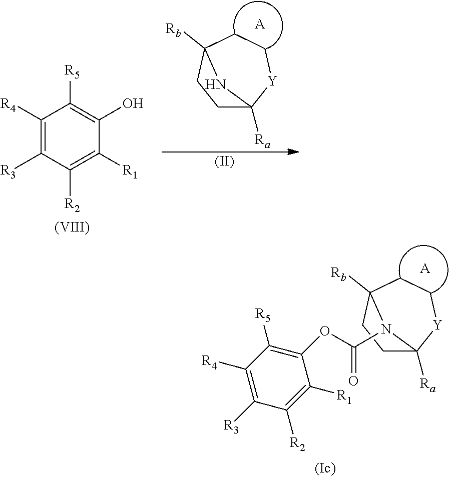

A further aspect of the invention relates to a process for preparing a compound as defined herein wherein p is 1, q is 1, r is 0, X is NH, Z is CR₁₈ and R₁₈ is NO₂, said process comprising treating a compound of formula (III), where R₁-R₅ are as defined above, with a compound of formula (IX) (CAS:13623-94-4) in suitable solvent (e.g. EtOH) to form a compound of formula (X), and then reacting said compound of formula (X) with a compound of formula (II) (for example, in EtOH). Suitable reaction conditions are described, for example, in WO2005/066130 (Euro Celtique SA).

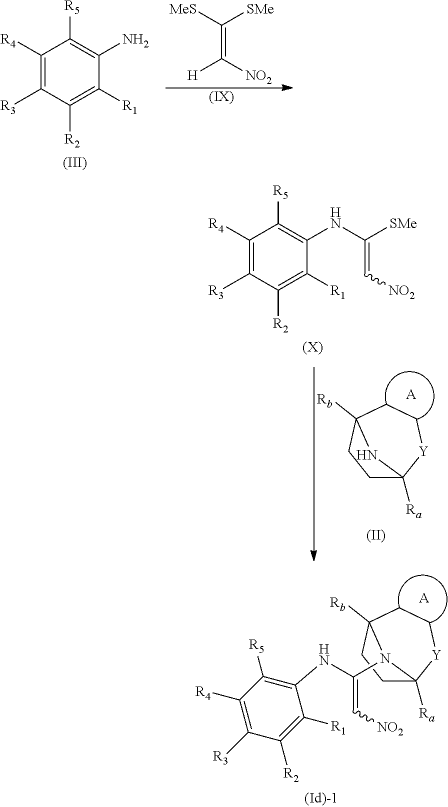

A further aspect of the invention relates to a process for preparing a compound as defined herein wherein p is 1, q is 1, r is 0, X is NH, Z is NR₁₇ and R₁₇ is H, said process comprising reacting benzotriazole with cyanogen bromide to form a compound of formula (XI), and then reacting said compound of formula (XI) with a compound of formula (II) in a suitable solvent (e.g. THF) to form a compound of formula (XII) Said compound of formula (XII) can then be reacted with a compound of formula (III), wherein R₁-R₅ are as defined herein. Further details of suitable reaction conditions and reagents are described in J. Org. Chem. 2000, 65, 8080-8082; Katritzky et al.

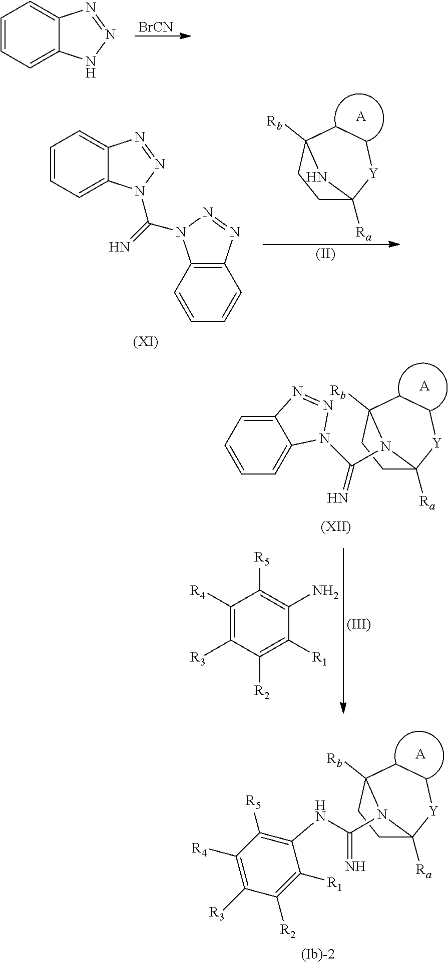

A further aspect of the invention relates to a process for preparing a compound as defined herein wherein p is 1, q is 1, r is 0, X is NH, Z is NR₁₇ and R₁₇ is OH or alkoxy, said process comprising reacting a compound of formula (IV) with a compound of formula (II), for example, in accordance with the reaction conditions and methodology described in WO2007/095050 (Incyte Corporation). Typical conditions include treating with Mel, K₂CO₃, followed by NH₂OH in water. The N-hydroxyguanidine group can then be optionally alklylated (for example, by treating with NaH/Mel) to give the corresponding alkoxy derivative, i.e. where R₁₇ is alkoxy (see, for example, the reaction conditions and methodology described in WO2004/029031; Euro Celtique SA).

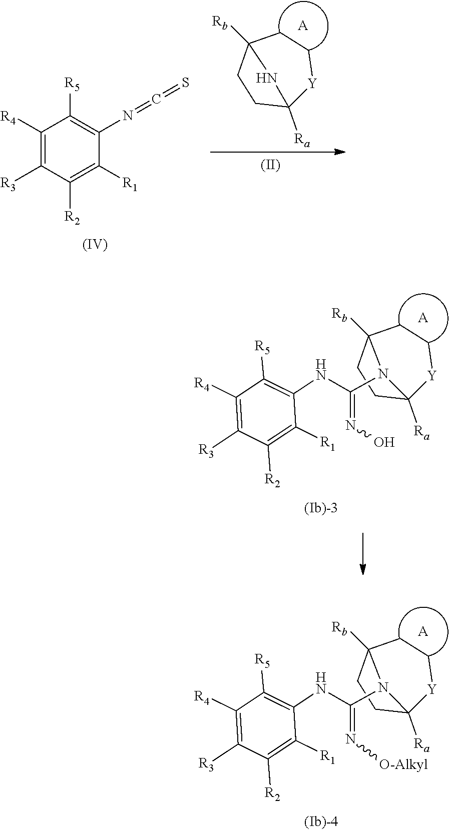

A further aspect of the invention relates to a process for preparing a compound as defined herein wherein p is 1, q is 1, r is 0, X is NH, Z is NR₁₇ and R₁₇ is H, OH or alkoxy, said process comprising reacting a compound of formula (Ia) with Mel and sodium hydride to form a compound of formula (XIII) in a suitable solvent (preferably DMF). Said compound of formula (XIII) can then be converted into a compound of formula (Ib)-2 by treating said compound of formula (XIII) with NH₃ in methanol and aqueous NH₄Cl, preferably under microwave conditions. Preferably, the reaction is carried out in a mixture of DMAP and EtOH.

Alternatively, said compound of formula (XIII) can converted into a compound of formula (Ib)-3 by treating said compound of formula (XIII) with NH₂OH in water, preferably in the presence of a suitable solvent (for example, EtOH).

Alternatively, said compound of formula (XIII) can converted into a compound of formula (Ib)-4 by treating said compound of formula (XIII) in the presence of pyridine/EtOH with NH₂O-alkyl hydrochloride (e.g. O-methylhydroxylamine hydrochloride).

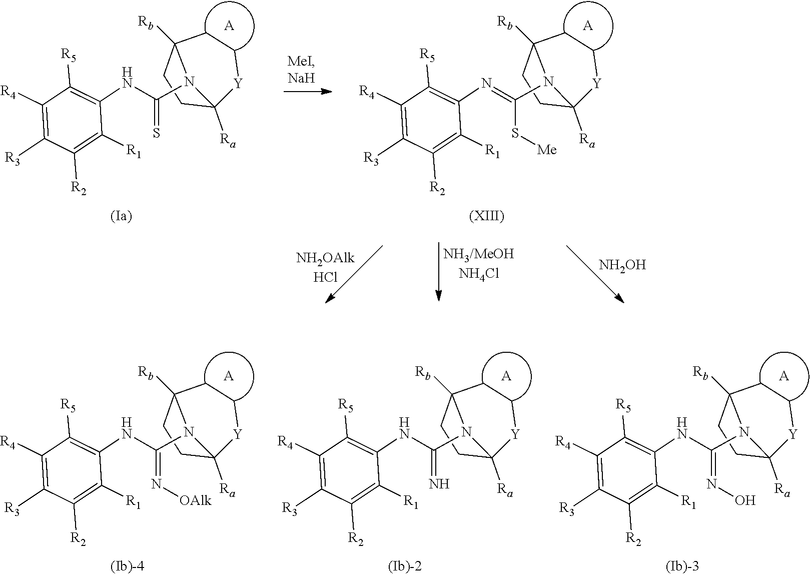

### Therapeutic Applications

A further aspect of the invention relates to compounds as described herein for use in medicine. The compounds have particular use in the field of oncology, immuno-oncology, and immunology as described in more detail below. In a preferred embodiment, the compound of the invention modulates GPR65, and more preferably inhibits GPR65 signalling.

A further aspect of the invention relates to a compound of formula (Ih), or a pharmaceutically acceptable salt or solvate thereof,

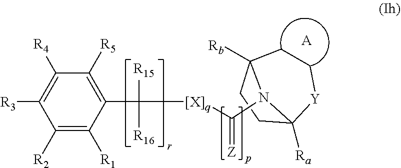

wherein:
ring A is a 5 or 6 membered aromatic or heteroaromatic ring, wherein said aromatic or heteroaromatic ring is optionally substituted with one or more substituents selected from halo, CN, alkoxy, NR₁₁R_{11′}, OH, CONR₁₉R₂₀, SO₂NR₁₉R₂₀, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R_{11′}, OH, alkyl, haloalkyl, and aralkyl;
Y is selected from CH₂, C═N—OH, and CR₁₀R_{10′};
Rₐ and R_{b} are each independently selected from H and alkyl;
Z is selected from O, S, NR₁₇ and CR₁₃;
X is selected from O and NH;
p is 0, 1 or 2;
q is 0 or 1; and
r is 0 or 1;
wherein at least one of p, q and r is other than zero, and with the proviso that:
(i) when r is 0, p is 1 and q is 1, when X is NH, Z is other than O;
(ii) when r and q are both 0, and p is 1, Z is not O;
R₁, R₄, and R₅ are each independently selected from H, alkyl, alkoxy, OH, F, C, Br, and I;
R₂ and R₃ are each independently selected from H, F, C, Br, I, CN, methoxy, haloalkyl, haloalkoxy and CO₂-alkyl;
R₁₀ and R_{10′} are each independently selected from H, F, alkyl, and haloalkyl;
R₁₁ and R_{11′} are each independently selected from H, alkyl, haloalkyl, COR₁₂, CO₂R₁₂ and SO₂R₁₃;
R₁₂ and R₁₃ are each independently alkyl;
R₁₅ and R₁₆ are each independently selected from H, alkoxy, alkyl and OH;
R₁₇ is selected from H, CN, OH, alkoxy and alkyl;
R₁₈ is NO₂ or CN;
each R₁₉ is independently H or alkyl; and
each R₂₀ is independently alkyl;
for use as a medicament.

Preferred definitions for A, X, Y, Z, p, q, r, Rₐ, R_{b} and R₁-R₅ are as set out above for compounds of formulae (I) and (1a)-(Ig).

Preferably, the compound of formula (Ih) is for use in treating or preventing a disorder selected from a proliferative disorder, an immune disorder, asthma, chronic obstructive pulmonary disease (COPD) and acute respiratory distress syndrome (ARDS).

Another aspect of the invention relates to compounds as described herein for use as a medicament, preferably for use in treating or preventing a disorder selected from a proliferative disorder and an immune disorder.

Another aspect of the invention relates to compounds as described herein for use in treating or preventing asthma and/or chronic obstructive pulmonary disease (COPD). GPR65 variant/SNP (rs6574978) has been shown to be associated with asthma/COPD syndrome with almost GWAS significant p value (1.18×10 e-7) (Hardin, 2014). Furthermore, GPR65 activation by pH (pH is low/acidic in asthmatic lungs) promotes eosinophil viability in a cAMP-dependent manner, contributing to disease progression/exacerbation. It is further known that GPR65 KO mice have attenuated asthma symptoms (Kottyan, 2009).

Another aspect of the invention relates to compounds as described herein for use in treating or preventing acute respiratory distress syndrome (ARDS). GPR65 has been shown to be protective in a model of LPS-induced acute lung injury model (Tsurumaki, 2015).

One aspect of the invention relates to a compound as described herein for use in treating a proliferative disorder. Preferably, the proliferative disorder is a cancer or leukemia.

In one preferred embodiment, the cancer is a solid tumour and/or metastases thereof.

In another preferred embodiment, the cancer is selected from melanoma, renal cell carcinoma (RCC), gastric cancer, acute myeloid leukaemia (AML), triple negative breast cancer (TNBC), head and neck cancer, colorectal cancer, colorectal adenocarcinoma, pancreatic adenocarcinoma, lung cancer, sarcoma, ovarian cancer, and gliomas, preferably glioblastoma (GBM).

Without wishing to be bound by theory, it is understood that GPR65 modulators are capable of preventing the increase in cytoplasmic cAMP in tumour-associated macrophages (TAMs) that would typically result from their exposure to the acidic tumour microenvironment and concomitant GPR65 activation. This reduction in the level of cytoplasmic cAMP in turn reduces the levels of ICER and TNFα, preventing the polarization of TAMs that are associated with non-inflammatory and tumour-permissive environment. Therefore, GPR65 modulators are expected to result in an increase in the visibility of the tumour to the immune system leading to increased immune-mediated tumour clearance. This suggests that modulation of GPR65 activity could be an effective treatment for cancer as stand-alone therapy or in combination with cancer immunotherapies (vaccines, agents that promote T cell mediated immune responses) or in patients that do not respond to immunomodulatory approaches such as PD1/PDL-1 blockade.

Another aspect of the invention relates to a compound as described herein for use in treating or preventing an immune disorder, preferably an autoimmune disease.

In one embodiment, the autoimmune disease is selected from psoriasis, psoriatic arthritis, rheumatoid arthritis (RA), multiple sclerosis (MS), systemic lupus erythematosus (SLE), autoimmune thyroiditis (Hashimoto's thyroiditis), Graves' disease, uveitis (including intermediate uveitis), ulcerative colitis, Crohn's disease, autoimmune uveoretinitis, systemic vasculitis, polymyositis-dermatomyositis, systemic sclerosis (scleroderma), Sjogren's Syndrome, ankylosing spondylitis and related spondyloarthropathies, sarcoidosis, autoimmune hemolytic anemia, immunological platelet disorders, autoimmune polyendocrinopathies, autoimmune myocarditis, type I diabetes and atopic dermatitis.

In a particularly preferred embodiment, the autoimmune disease is selected from psoriasis, psoriatic arthritis, ankylosing spondylitis, Crohn's disease, and multiple sclerosis (MS).

Without wishing to be bound by theory, it is understood that GPR65 modulators will prevent the upregulation of ICER in CD4+ T cells. This, in turn, is expected to prevent the ICER-associated suppression of IL-2 that biases CD4+ T cells toward the inflammatory Th17 phenotype associated with increased pathogenicity in the context of autoimmune disease. This is supported by the fact that mutations in the GPR65 locus are associated with several autoimmune diseases, such as multiple sclerosis, ankylosing spondylitis, inflammatory bowel disease, and Crohn's disease (Gaublomme, 2015). This suggests that modulation of GPR65 activity could be an effective treatment for autoimmune diseases.

Another aspect relates to a compound as described herein for use in treating or preventing a disorder caused by, associated with or accompanied by abnormal activity against GPR65.

Another aspect relates to a compound as described herein for use in treating or preventing a GPR65-associated disease or disorder.

Another aspect of the invention relates to a method of treating a disorder as described above comprising administering a compound as described herein to a subject.

Another aspect of the invention relates to a method of treating a GPR65-associated disease or disorder in a subject. The method according to this aspect of the present invention is effected by administering to a subject in need thereof a therapeutically effective amount of a compound of the present invention, as described hereinabove, either per se, or, more preferably, as a part of a pharmaceutical composition, mixed with, for example, a pharmaceutically acceptable carrier, as is detailed hereinafter.

Yet another aspect of the invention relates to a method of treating a subject having a disease state alleviated by modulation of GPR65 wherein the method comprises administering to the subject a therapeutically effective amount of a compound according to the invention.

Another aspect relates to a method of treating a disease state alleviated by modulation of GPR65, wherein the method comprises administering to a subject a therapeutically effective amount of a compound according to the invention.

Preferably, the subject is a mammal, more preferably a human.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein, the term “treating” includes abrogating, substantially inhibiting, slowing or reversing the progression of a disease or disorder, substantially ameliorating clinical symptoms of a disease or disorder or substantially preventing the appearance of clinical symptoms of a disease or disorder.

Herein, the term “preventing” refers to a method for barring an organism from acquiring a disorder or disease in the first place.

The term “therapeutically effective amount” refers to that amount of the compound being administered which will relieve to some extent one or more of the symptoms of the disease or disorder being treated.

For any compound used in this invention, a therapeutically effective amount, also referred to herein as a therapeutically effective dose, can be estimated initially from cell culture assays.

For example, a dose can be formulated in animal models to achieve a circulating concentration range that includes the IC₅₀ or the IC₁₀₀ as determined in cell culture. Such information can be used to more accurately to determine useful doses in humans. Initial dosages can also be estimated from in vivo data. Using these initial guidelines one of ordinary skill in the art could determine an effective dosage in humans.

Moreover, toxicity and therapeutic efficacy of the compounds described herein can be determined by standard pharmaceutical procedures in cell cultures or experimental animals, e.g., by determining the LD₅₀ and the ED₅₀. The dose ratio between toxic and therapeutic effect is the therapeutic index and can be expressed as the ratio between LD₅₀ and ED₅₀. Compounds which exhibit high therapeutic indices are preferred. The data obtained from these cell cultures assays and animal studies can be used in formulating a dosage range that is not toxic for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition (see, e.g., Fingl et al, 1975, The Pharmacological Basis of Therapeutics, chapter 1, page 1).

Dosage amount and interval may be adjusted individually to provide plasma levels of the active compound which are sufficient to maintain therapeutic effect. Usual patient dosages for oral administration range from about 50-2000 mg/day, commonly from about 100-1000 mg/day, preferably from about 150-700 mg/day and most preferably from about 250-500 mg/day or from 50-100 mg/day. Preferably, therapeutically effective serum levels will be achieved by administering multiple doses each day. In cases of local administration or selective uptake, the effective local concentration of the drug may not be related to plasma concentration. One skilled in the art will be able to optimize therapeutically effective local dosages without undue experimentation.

As used herein, “GPR65-related disease or disorder” refers to a disease or disorder characterized by inappropriate GPR65 activity. Inappropriate GPR65 activity refers to either an increase or decrease in GPR65 activity as measured by enzyme or cellular assays, for example, compared to the activity in a healthy subject. Inappropriate activity could also be due to overexpression of GPR65 in diseased tissue compared with healthy adjacent tissue.

Preferred diseases or disorders that the compounds described herein may be useful in preventing include proliferative disorders and immune disorders as described hereinbefore, as well as asthma and chronic obstructive pulmonary disease.

Thus, the present invention further provides use of compounds as defined herein in the preparation of a medicament for the treatment of a disease where it is desirable to modulate GPR65. Such diseases include proliferative disorders and immune disorders as described hereinbefore, as well as asthma and chronic obstructive pulmonary disease.

As used herein the phrase “preparation of a medicament” includes the use of the components of the invention directly as the medicament in addition to their use in any stage of the preparation of such a medicament.

In one preferred embodiment, the compound prevents the increase in cytoplasmic cAMP levels expected following GPR65 activation at acidic pH. This prevention of cAMP accumulation in turn prevents downstream signalling through ICER, as described above. The “Human GPR65 cyclic adenosine monophosphate (cAMP) Homogeneous Time Resolved Fluorescence (HTRF) antagonist assay”, or simply “cAMP assay”, as described in the accompanying examples, can be used to measure the potency of GPR65 modulators, which is expressed as the concentration of compound required to reduce the increase in cAMP concentration upon GPR65 activation by 50% (i.e. an IC₅₀).

In one preferred embodiment, the compound exhibits an IC₅₀ value in the aforementioned cAMP assay of less than about 25 μM. More preferably, the compound exhibits an IC₅₀ value in the cAMP assay of less than about 10 μM, more preferably, less than about 5 μM, even more preferably, less than about 1 μM, even more preferably, less than about 0.1 μM.

In another preferred embodiment, the compound exhibits an hGPR65 IC₅₀ value of less than <5 μM, more preferably less than <500 nM in the aforementioned assay.

In one preferred embodiment, the compound according to the invention exhibits an IC₅₀ of >500 nM and <5 μM in the aforementioned assay. In one preferred embodiment, the compound is selected from those denoted “high” or “medium” in Table 1.

In a more preferred embodiment, the compound according to the invention exhibits an IC₅₀ of <500 nM in the aforementioned assay. In one preferred embodiment, the compound is selected from those denoted “high” in Table 1.

### Pharmaceutical Compositions

For use according to the present invention, the compounds or physiologically acceptable salt, ester or other physiologically functional derivative thereof, described herein, may be presented as a pharmaceutical formulation, comprising the compounds or physiologically acceptable salt, ester or other physiologically functional derivative thereof, together with one or more pharmaceutically acceptable carriers, excipients or diluents therefor and optionally other therapeutic and/or prophylactic ingredients. The carrier(s) must be acceptable in the sense of being compatible with the other ingredients of the formulation and not deleterious to the recipient thereof. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller. The carrier, or, if more than one be present, each of the carriers, must be acceptable in the sense of being compatible with the other ingredients of the formulation and not deleterious to the recipient.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s), buffer(s), flavouring agent(s), surface active agent(s), thickener(s), preservative(s) (including anti-oxidants) and the like, and substances included for the purpose of rendering the formulation isotonic with the blood of the intended recipient.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release—controlling matrix, or is coated with a suitable release—controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds. Pharmaceutical formulations suitable for parenteral administration include sterile solutions or suspensions of an active compound in aqueous or oleaginous vehicles.

Injectable preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

According to a further aspect of the invention, there is provided a process for the preparation of a pharmaceutical or veterinary composition as described above, the process comprising bringing the active compound(s) into association with the carrier, for example by admixture.

In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound as described herein into conjunction or association with a pharmaceutically or veterinarily acceptable carrier or vehicle.

### Salts/Esters

The compounds of the invention can be present as salts or esters, in particular pharmaceutically and veterinarily acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. hydrohalic acids such as hydrochloride, hydrobromide and hydroiodide, sulphuric acid, phosphoric acid sulphate, bisulphate, hemisulphate, thiocyanate, persulphate and sulphonic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Salts which are not pharmaceutically or veterinarily acceptable may still be valuable as intermediates.

Preferred salts include, for example, acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonates, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Enantiomers/Tautomers

In all aspects of the present invention previously discussed, the invention includes, where appropriate all enantiomers, diastereoisomers and tautomers of the compounds of the invention. The person skilled in the art will recognise compounds that possess optical properties (one or more chiral carbon atoms) or tautomeric characteristics. The corresponding enantiomers and/or tautomers may be isolated/prepared by methods known in the art.

Enantiomers are characterised by the absolute configuration of their chiral centres and described by the R- and S-sequencing rules of Cahn, Ingold and Prelog. Such conventions are well known in the art (e.g. see ‘Advanced Organic Chemistry’, 3^{rd} edition, ed. March, J., John Wiley and Sons, New York, 1985).

Compounds of the invention containing a chiral centre may be used as a racemic mixture, an enantiomerically enriched mixture, or the racemic mixture may be separated using well-known techniques and an individual enantiomer may be used alone.

### Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those compounds, and mixtures thereof. The terms used in the claims encompass these forms, provided said forms retain the appropriate functional activity (though not necessarily to the same degree).

The present invention also includes all suitable isotopic variations of the compound or a pharmaceutically acceptable salt thereof. An isotopic variation of a compound of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. For example, the invention includes compounds of general formula (I) where any hydrogen atom has been replaced by a deuterium atom. Isotopic variations of the agent of the present invention and pharmaceutically acceptable salts thereof of this invention can generally be prepared by conventional procedures using appropriate isotopic variations of suitable reagents.

### Atropisomers

Some of the compounds of the invention may exist as atropisomers. Atropisomers are stereoisomers arising because of hindered rotation about a single bond, where energy differences due to steric strain or other contributors create a barrier to rotation that is high enough to allow for isolation of individual conformers. The invention encompasses all such atropisomers.

### Prodrugs

The invention further includes the compounds of the present invention in prodrug form, i.e. covalently bonded compounds which release the active parent drug in vivo. Such prodrugs are generally compounds of the invention wherein one or more appropriate groups have been modified such that the modification may be reversed upon administration to a human or mammalian subject. Reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Solvates

The present invention also includes solvate forms of the compounds of the present invention. The terms used in the claims encompass these forms. Preferably, the solvate is a hydrate.

### Combinations

A further aspect of the invention relates to a combination comprising a compound as described herein and one or more additional active agents. In a particularly preferred embodiment, the one or more compounds of the invention are administered in combination with one or more additional active agents, for example, existing drugs available on the market. In such cases, the compounds of the invention may be administered consecutively, simultaneously or sequentially with the one or more other active agents.

Drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance.

Beneficial combinations may be suggested by studying the activity of the test compounds with agents known or suspected of being valuable in the treatment of a particular disorder. This procedure can also be used to determine the order of administration of the agents, i.e. before, simultaneously, or after delivery. Such scheduling may be a feature of all the active agents identified herein.

In the context of cancer, compounds of the invention can be used in combination with immunotherapies such as cancer vaccines and/or with other immune-modulators such as agents that block the PD1/PDL-1 interaction. Other examples of agents for use in combination with the presently claimed compounds include immune modulators that block CTLA-4 or LAG-3. Thus, in one preferred embodiment, the additional active agent is an immunotherapy agent, more preferably a cancer immunotherapy agent. An “immunotherapy agent” refers to a treatment that uses the subject's own immune system to fight diseases such as cancer. For other disorders the compounds of the invention can be used in combination agents that block or decrease inflammation such as antibodies that target pro-inflammatory cytokines. The compounds of the invention can also be used in combination with other chemotherapy agents and/or in conjunction with radiotherapy.

### Polymorphs

The invention further relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Administration

The pharmaceutical compositions of the present invention may be adapted for rectal, nasal, intrabronchial, topical (including buccal and sublingual), vaginal or parenteral (including subcutaneous, intramuscular, intravenous, intraarterial and intradermal), intraperitoneal or intrathecal administration. Preferably the formulation is an orally administered formulation. The formulations may conveniently be presented in unit dosage form, i.e., in the form of discrete portions containing a unit dose, or a multiple or sub-unit of a unit dose. By way of example, the formulations may be in the form of tablets and sustained release capsules, and may be prepared by any method well known in the art of pharmacy.

Formulations for oral administration in the present invention may be presented as: discrete units such as capsules, gellules, drops, cachets, pills or tablets each containing a predetermined amount of the active agent; as a powder or granules; as a solution, emulsion or a suspension of the active agent in an aqueous liquid or a non-aqueous liquid; or as an oil-in-water liquid emulsion or a water-in-oil liquid emulsion; or as a bolus etc. Preferably, these compositions contain from 1 to 250 mg and more preferably from 10-100 mg, of active ingredient per dose.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropyl-methylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Other forms of administration comprise solutions or emulsions which may be injected intravenously, intraarterially, intrathecally, subcutaneously, intradermally, intraperitoneally or intramuscularly, and which are prepared from sterile or sterilisable solutions. Injectable forms typically contain between 10-1000 mg, preferably between 10-250 mg, of active ingredient per dose.

The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

The dosage amount will further be modified according to the mode of administration of the compound. For example, to achieve an “effective amount” for acute therapy, parenteral administration of a compound is typically preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg; preferably between 0.1 and 20 mg, in a manner to maintain the concentration of drug in the plasma at a concentration effective to modulate GPR65. The compounds may be administered one to four times daily at a level to achieve a total daily dose of about 0.4 to about 400 mg. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to achieve one or more of the therapeutic indications disclosed herein. Typically, a pharmaceutical composition containing the compound is administered at an oral dose of between about 0.1 to about 500 mg or about 0.1 to about 50 mg in a manner consistent with the condition of the patient. Preferably the oral dose would be about 0.5 to about 50 mg or about 0.5 to about 20 mg.

No unacceptable toxicological effects are expected when compounds of the present invention are administered in accordance with the present invention. The compounds of this invention, which may have good bioavailability, may be tested in one of several biological assays to determine the concentration of a compound which is required to have a given pharmacological effect.

The invention is further described by way of the following non-limiting examples.

### EXAMPLES

Where the preparation of starting materials is not described, these are commercially available, known in the literature, or readily obtainable by those skilled in the art using standard procedures. Where it is indicated that compounds were prepared analogously to earlier examples or intermediates, it will be appreciated by the skilled person that the reaction time, number of equivalents of reagents, solvent, concentration and temperature can be modified for each specific reaction and that it may be necessary or desirable to employ different work-up or purification techniques.

### General Schemes

### Abbreviations

A list of some common abbreviations is shown below—where other abbreviations are used which are not listed, these will be understood by the person skilled in the art.

CAN: ceric ammonium nitrate; d: doublet; DCM: dichloromethane; DIPEA: N,N-diisopropylethylamine; DMF: N,N-dimethylformamide; DMSO: dimethylsulfoxide; (ES+): electrospray ionization positive mode; Et₃N: triethylamine; EtOAc: ethyl acetate; h: hours; HPLC: high performance liquid chromatography; Hz: hertz; IPA: iso-propyl alcohol; J: coupling constant; I: litre; M: molar; m: multiplet [M+H]+: protonated molecular ion; MeCN: acetonitrile; MeOH: methanol; MHz: megahertz; min: minutes; ml: millilitres; MS: mass spectrometry; MTBE: methyl tert-butyl ether; m/z: mass-to-charge ratio; NMR: nuclear magnetic resonance; Pd-161: chloro(crotyl)[(p-dimethylaminophenyl)(di-tert-butylphosphine)]palladium(II); Pd-178: chloro(crotyl)(tricyclohexylphosphine)palladium(II); PDA: photodiode array; PMP: para-methoxyphenyl; RT: room temperature; Rt: retention time; s: singlet; SCX: solid supported cation exchange (resin); t: triplet; THF: tetrahydrofuran; TLC: thin layer chromatography; UPLC: ultra performance liquid chromatography; UV: ultra-violet.

Other abbreviations are intended to convey their generally accepted meaning.

### General Experimental Conditions

All starting materials and solvents were obtained either from commercial sources or prepared according to literature methods. The appropriate isocyanate and aniline starting materials were obtained from Sigma Aldrich, Fluorochem or Enamine store, or were synthesised as described herein. The appropriate tricyclic amine starting materials were obtained from Enamine store or were synthesised as described herein. Reaction mixtures were magnetically stirred and reactions performed at room temperature (approximately 20° C.) unless otherwise indicated.

Silica gel chromatography was performed on an automated flash chromatography system, such as CombiFlash Companion, CombiFlash Rf system or Reveleris X2 flash system using RediSep® Rf or Reveleris® or the GraceResolv™ pre-packed silica (230-400 mesh, 40-63 μm) cartridges.

Analytical UPLC-MS experiments to determine retention times and associated mass ions were performed using a Waters ACQUITY UPLC® H-Class system, equipped with ACQUITY PDA Detector and ACQUITY QDa mass spectrometer or Waters SQD mass spectrometer, running the analytical method described below.

Analytical LC-MS experiments to determine retention times and associated mass ions were performed using an Agilent 1200 series HPLC system coupled to an Agilent 1956, 6100 or 6120 series single quadrupole mass spectrometer running one of the analytical methods described below.

Preparative HPLC purifications were performed either using either a Waters X-Bridge BEH C18, 5 μm, 19×50 mm column, or a Waters Xbridge Prep OBD C18, 10 μm, 40×150 mm column, or a Phenomenex Gemini-NX C18, 3 μm, 30×75 mm column using a gradient of MeCN and 10 mM aqueous ammonium bicarbonate. Fractions were collected following UV detection across all wavelengths with PDA and in some cases an SQD2 or ACQUITY QDa mass spectrometer.

NMR spectra were recorded using either a Bruker Avance III HD 500 MHz instrument or a Bruker Avance Neo 400 MHz, using either residual non-deuterated solvent or tetra-methylsilane as reference or Varian Y 400 MHz instrument, using tetra-methylsilane as reference.

In the absence of the absolute stereochemistry being explicitly indicated through wedged and dashed bonds, chemical structures disclosed throughout the examples are to be interpreted as depicting the racemate. For the avoidance of doubt, the invention encompasses the compounds in either configuration, as well as mixtures thereof.

### Analytical Methods

### Method 1—Basic 3 Min Method

Column: Waters ACQUITY UPLC® BEH C18, 1.7 μm, 2.1×30 mm at 40° C.

Detection: UV at 210-400 nm unless otherwise indicated, MS by electrospray ionisation

Solvents: A: 10 mM aqueous ammonium bicarbonate, B: MeCN

Gradient:

| Time | % A | % B | Flow rate (ml/min) |
|---|---|---|---|
| 0.00 | 95 | 5 | 0.77 |
| 0.11 | 95 | 5 | 0.77 |
| 2.15 | 5 | 95 | 0.77 |
| 2.56 | 5 | 95 | 0.77 |
| 2.83 | 95 | 5 | 0.77 |
| 3.00 | 95 | 5 | 0.77 |

### Method 2—Basic 4 Min Method

Column: Waters X-Bridge BEH C18, 2.5 μm, 4.6×30 mm at 40° C.

Detection: UV at 254 nm unless otherwise indicated, MS by electrospray ionisation

Solvents: A: 10 mM ammonium bicarbonate(aq), B: MeCN

Gradient:

| Time | % A | % B | Flow rate (ml/min) |
|---|---|---|---|
| 0.0 | 95.0 | 5.0 | 2.5 |
| 3.0 | 5.0 | 95.0 | 2.5 |
| 3.01 | 5.0 | 95.0 | 4.5 |
| 3.6 | 5.0 | 95.0 | 4.5 |
| 3.7 | 95.0 | 5.0 | 2.5 |
| 4.0 | 95.0 | 5.0 | 2.5 |

### Method 3—Acidic 3 Min Method

Column: Waters CSH C18, 1.7 μm, 2.1×30 mm at 40° C.

Detection: UV at 210-400 nm unless otherwise indicated, MS by electrospray ionisation

Solvents: A: 0.1% Formic acid in water, B: MeCN

Gradient:

| Time | % A | % B | Flow rate (ml/min) |
|---|---|---|---|
| 0.00 | 98 | 2 | 0.77 |
| 2.50 | 0 | 100 | 0.77 |
| 3.00 | 0 | 100 | 0.77 |

### Method 4—Acidic 3 Min Method 2

Column: Waters Cortecs C18, 30×2.1 mm, 2.7 μm at 40° C.

Detection: UV at 210-400 nm unless otherwise indicated, MS by electrospray ionisation

Solvents: A: 0.1% Formic acid in water, B: MeCN

Gradient:

| Time | % A | % B | Flow rate (ml/min) |
|---|---|---|---|
| 0.00 | 95 | 5 | 1.35 |
| 2.50 | 0 | 100 | 1.35 |
| 3.00 | 0 | 100 | 1.35 |

### Experimental scheme 1

### Compound 1 (5R,8S)—N-(5-Bromo-2-fluoro-4-(trifluoromethyl)phenyl)-1-fluoro-6,7,8,9-tetrahydro-5H-5,8-epiminocyclohepta[c]pyridine-10-carbothioamide

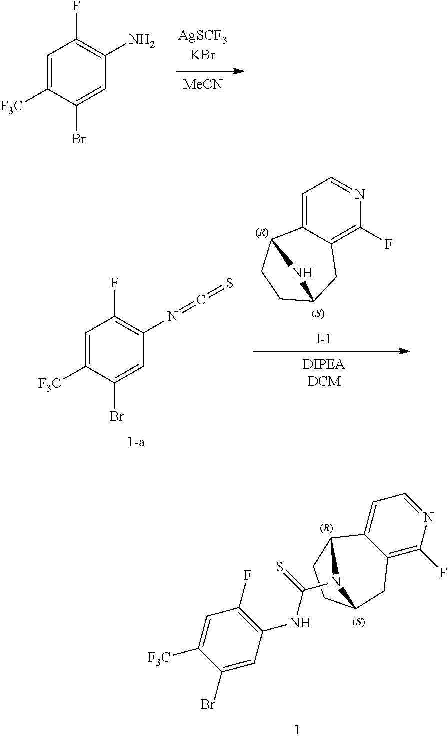

Step 1: To a solution of silver trifluoromethanethiolate (176 mg, 842 μmol) in MeCN (3 ml), under a nitrogen atmosphere, potassium bromide (100 mg, 842 μmol) was added, followed by a solution of 5-bromo-2-fluoro-4-(trifluoromethyl)aniline (130 mg, 505 μmol) in MeCN (3 ml). Light was excluded and the mixture was stirred at RT for 48 h. The reaction mixture was diluted with more MeCN (5 ml) and filtered through a Celite pad. The cake was washed with more MeCN (10 ml). The filtrate was concentrated in vacuo. The product was purified by chromatography on silica gel (100% isohexane) to afford 1-bromo-4-fluoro-5-isothiocyanato-2-(trifluoromethyl)benzene) as a yellow liquid. ¹H NMR (400 MHz, CDCl₃) δ 7.57-7.44 (m, 2H). ¹⁹F NMR (376 MHz, CDCl₃) δ −62.8 (3F), −119.2 (1F).

Step 2: To a solution of 1-bromo-4-fluoro-5-isothiocyanato-2-(trifluoromethyl)benzene) (84 mg, 260 mol) in DCM (1 ml) and added to a solution of (5R,8S)-1-fluoro-6,7,8,9-tetrahydro-5H-5,8-epiminocyclohepta[c]pyridine I-1 (75 mg, 421 μmol) and DIPEA (73 μl, 421 μmol) in DCM (1 ml). The reaction was stirred at RT for 16 h. The reaction mixture was diluted with 10% MeOH in DCM (5 mL) and then concentrated in vacuo. The product was purified by chromatography on silica gel (0-100% EtOAc/isohexane) to afford (5R,8S)—N-(5-bromo-2-fluoro-4-(trifluoromethyl)phenyl)-1-fluoro-6,7,8,9-tetrahydro-5H-5,8-epiminocyclohepta[c]pyridine-10-carbothioamide as an off-white solid. LCMS (method 1) m/z 475.9, 477.9 (M−H)⁻ (ES⁻), at 1.28 min. ¹H NMR (400 MHz, DMSO-d6) δ 9.65 (s, 1H), 8.04 (d, J=5.0 Hz, 1H), 7.91 (s, 1H), 7.81 (d, J=10.3 Hz, 1H), 7.22 (s, 1H), 5.78 (s, 1H), 5.17 (s, 1H), 3.42-3.31 (m, 1H), 3.29 (s, 1H), 2.68 (d, J=17.4 Hz, 1H), 2.45-2.21 (m, 1H), 2.02-1.78 (m, 2H).

The following compounds were prepared using appropriate starting materials in an analogous procedure to that described in Experimental Scheme 1. Where the starting materials are not described in the literature, their synthesis is described below.

Key: 1: [M−H]⁻ reported as [M+H]⁺ not observed

| | | LCMS | |
|---|---|---|---|
| | | method 1 | |
| Compound | Structure | Rt [M + H]⁺ | NMR |
| 2 | 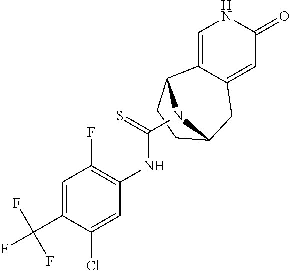 [SMILES: O=c1cc2c(c[nH]1)[C@H]1CC[C@@H](C2)N1C(=S)Nc1cc(Cl)c(C(F)(F)F)cc1F] | 430.1, 432.2 at 1.00 min | ¹H NMR (400 MHz, DMSO-d6) δ 11.36 (s, 1H), 9.46 (s, 1H), 7.83 (d, J = 10.1 Hz, 1H), 7.77 (d, J = 6.6 Hz, 1H), 7.32-7.12 (m, 1H), 6.15 (s, 1H), 5.79-5.33 (m, 1H), 5.21 − 4.79 (m, 1H), 3.30 (s, 1H), 2.64 (d, J = 18.6 Hz, 1H), 2.41 − 2.00 (m, 2H), 1.90-1.65 (s, 2H) |
| | (6S,9R)-N-(5-Chloro-2-fluoro-4- | | |
| | (trifluoromethyl)phenyl)-3-oxo- | | |
| | 3,5,6,7,8,9-hexahydro-2H-6,9- | | |
| | epiminocyclohepta[c]pyridine- | | |
| | 10-carbothioamide | | |
| 3 | 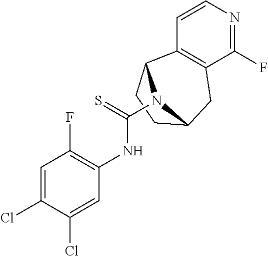 [SMILES: Fc1cc(Cl)c(Cl)cc1NC(=S)N1[C@H]2CC[C@@H]1c1ccnc(F)c1C2] | 398.1, 400.1 at 1.54 min¹ | ¹H NMR (400 MHz, DMSO-d6) δ 9.48 (s, 1H), 8.03 (d, J = 5.0 Hz, 1H), 7.78-7.54 (dd, J = 20.7, 8.3 Hz, 2H), 7.21 (d, J = 5.0 Hz, 1H), 5.77 (s, 1H), 5.16 (s, 1H), 3.41-3.26 (m, 1H), 2.66 (d, J = 17.3 Hz, 1H), 2.45-2.18 (m, 2H), 2.01-1.75 (m, 2H) |
| | (5R,8S)-N-(4,5-dichloro-2- | | |
| | fluorophenyl)-1-fluoro-6,7,8,9- | | |
| | tetrahydro-5H-5,8-epiminocyclo- | | |
| | hepta[c]pyridine-10- | | |
| | carbothioamide | | |
| 4 | 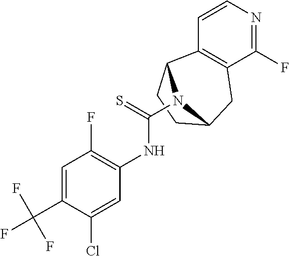 [SMILES: Fc1cc(C(F)(F)F)c(Cl)cc1NC(=S)N1[C@H]2CC[C@@H]1c1ccnc(F)c1C2] | | ¹H NMR (400 MHz, DMSO-d6) δ 9.66 (s, 1H), 8.04 (d, J = 5.0 Hz, 1H), 7.87-7.72 (m, 2H), 7.22 (s, 1H), 5.78 (s, 1H), 5.18 (s, 1H), 3.34-3.28 (m, 1H), 2.69 (d, J = 17.1 Hz, 1H), 2.46-2.19 (t, J = 1.9 Hz, 2H), 2.04-1.77 (s, 2H) |
| | (5R,8S)-N-(5-chloro-2-fluoro-4- | | |

(trifluoromethyl)phenyl)-1-fluoro-
6,7,8,9-tetrahydro-5H-5,8-
epiminocyclohepta[c]pyridine-
10-carbothioamide

### Intermediate 1 (I-1)

The following scheme makes use of synthetic methodology described in Schultz and Wolfe, Organic Letters, 2011, 13 (11), 2962-2965.

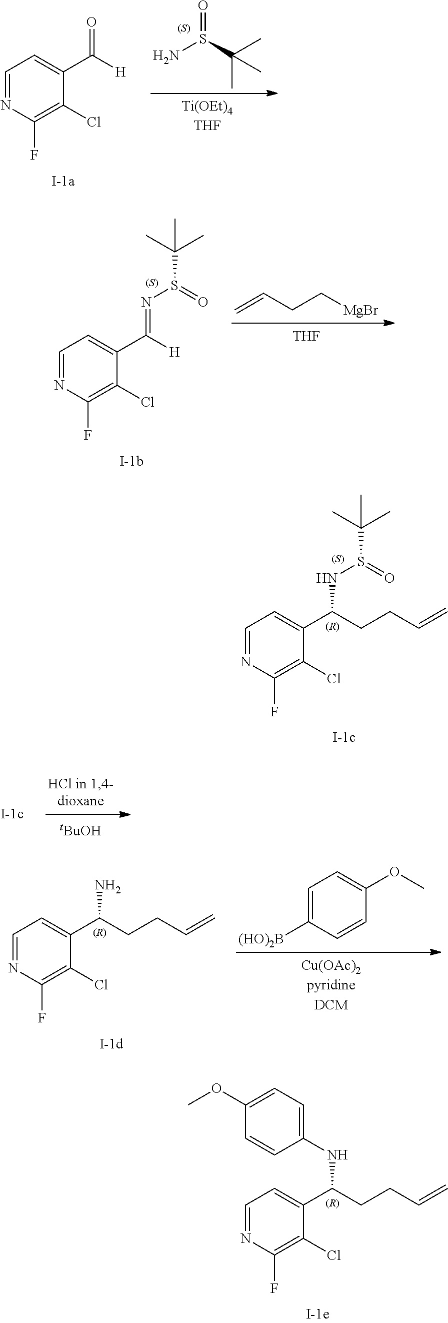

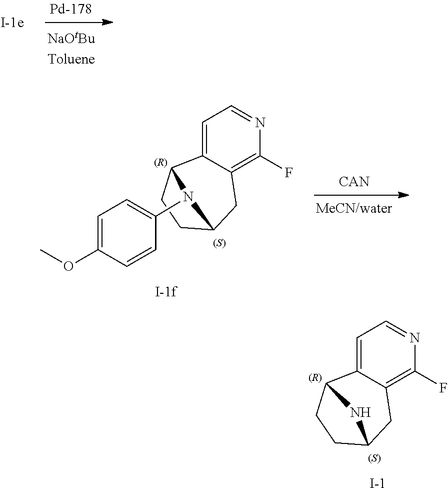

Step 1: To a solution of 3-chloro-2-fluoroisonicotinaldehyde I-1a (1.00 g, 6.27 mmol) in THF (17 ml) was added titanium (IV) ethoxide (2.63 ml, 12.5 mmol) in one portion at RT. The mixture was stirred at RT for 5 min before (S)-tert-butylsulfinamide (760 mg, 6.27 mmol) was added in one portion. The resulting mixture was stirred at RT for 16 h. Brine (30 ml) was added, and the mixture stirred for 10 minutes then filtered through celite. The filtrate was extracted with EtOAc (2×20 ml). The combined organic layers were dried over magnesium sulfate and concentrated in vacuo to give (S,E)-N-((3-chloro-2-fluoropyridin-4-yl)methylene)-2-methylpropane-2-sulfinamide (I-1b) as a pale yellow solid. LCMS (method 1) m/z 227.5 (M-Cl)⁺ (ES+), at 1.36 min. ¹H NMR (500 MHz, DMSO-d6) δ 8.81 (s, 1H), 8.33 (dt, J=5.1, 0.8 Hz, 1H), 7.90 (d, J=5.1 Hz, 1H), 1.22 (s, 9H).

Step 2: To a solution of (S,E)-N-((3-chloro-2-fluoropyridin-4-yl)methylene)-2-methylpropane-2-sulfinamide (1.3824 g, 5.2617 mmol) (I-1b) in THF (22 ml) was added but-3-en-1-yl magnesium bromide (0.5 M, 31.57 ml, 15.79 mmol) dropwise at −78° C. The mixture was warmed to RT slowly and stirred for 72 h. Saturated ammonium chloride solution (10 ml) was added and the product was extracted with EtOAc (3×10 ml). The combined organics were dried with magnesium sulfate and concentrated in vacuo. The product was purified by silica gel chromatography (0-100% EtOAc/isohexane) to give (S)—N—((R)-1-(3-chloro-2-fluoropyridin-4-yl)pent-4-en-1-yl)-2-methylpropane-2-sulfinamide (I-1c) as a pale yellow solid. LCMS (method 1) m/z 316.7, 319.1 (M−H)⁻ (ES⁻), at 1.39 min. 1H NMR (500 MHz, DMSO-d6) δ 8.21 (dd, J=5.2, 0.8 Hz, 1H), 7.53 (d, J=5.2 Hz, 1H), 5.90 (d, J=7.3 Hz, 1H), 5.81 (dddd, J=17.3, 10.2, 7.1, 6.1 Hz, 1H), 5.10 (dq, J=17.2, 1.7 Hz, 1H), 5.02 (ddt, J=10.2, 2.3, 1.2 Hz, 1H), 4.66 (ddd, J=8.6, 7.3, 5.3 Hz, 1H), 2.27-2.08 (m, 2H), 1.97-1.87 (m, 1H), 1.80-1.70 (m, 1H), 1.07 (s, 9H).

Step 3: To a solution of (S)—N—((R)-1-(3-chloro-2-fluoropyridin-4-yl)pent-4-en-1-yl)-2-methylpropane-2-sulfinamide (I-1c) (714 mg, 2.015 mmol) in ^{t}BuOH (7.2 ml) was added a solution of HCl in 1,4-dioxane (4 M, 3.0 ml, 12.09 mmol) at RT and stirred for 2.5 h. The reaction mixture was quenched with saturated aqueous sodium bicarbonate solution (100 ml) and extracted with DCM (3×30 ml). The organic layers were combined, dried over magnesium sulfate and concentrated in vacuo to give (R)-1-(3-chloro-2-fluoropyridin-4-yl)pent-4-en-1-amine as a pale yellow liquid (I-1d) as an orange/brown oil. LCMS (method 1): m/z 215.4, 217.3 (M+H)+(ES+); at 1.17 min, 1H NMR (500 MHz, DMSO-d6) δ 8.16 (dd, J=5.1, 0.9 Hz, 1H), 7.62 (d, J=5.1 Hz, 1H), 5.80 (ddt, J=16.9, 10.2, 6.6 Hz, 1H), 5.02 (dq, J=17.4, 1.8 Hz, 1H), 4.96 (ddt, J=10.2, 2.3, 1.3 Hz, 1H), 4.22 (dd, J=8.1, 5.1 Hz, 1H), 2.23-2.01 (m, 2H), 1.76-1.50 (m, 2H), 2 NH protons not observed.

Step 4: To a solution of (R)-1-(3-chloro-2-fluoropyridin-4-yl)pent-4-en-1-amine (I-1d) (644.3 mg, 3.001 mmol), (4-methoxyphenyl)boronic acid (1.37 g, 9.0 mmol), and Cu(OAc)₂ (820 mg, 4.50 mmol) in DCM (100 ml) was added pyridine (1.2 ml, 15.0 mmol) dropwise. The mixture was stirred at RT open to air for 16 h. 2M NaOH aqueous solution (20 ml) was added followed by water (20 ml) and the mixture extracted with DCM (3×20 ml). The combined organics were dried over magnesium sulfate and concentrated in vacuo. The product was purified by silica gel chromatography (0%-100% DCM/isohexane) to give (R)—N-(1-(3-chloro-2-fluoropyridin-4-yl)pent-4-en-1-yl)-4-methoxyaniline (I-1e) as a yellow oil.

LCMS (method 1): m/z 321.1, 323.4 (M+H)+(ES+); at 1.73 min, ¹H NMR (500 MHz, DMSO-d6) δ 8.10 (d, J=5.1 Hz, 1H), 7.41 (d, J=5.1 Hz, 1H), 6.65 (d, J=8.9 Hz, 2H), 6.38 (d, J=8.9 Hz, 2H), 6.09 (d, J=8.3 Hz, 1H), 5.84 (ddt, J=17.0, 10.2, 6.6 Hz, 1H), 5.10-4.88 (m, 2H), 4.68 (td, J=8.5, 4.7 Hz, 1H), 3.57 (s, 3H), 2.34-2.24 (m, 1H), 2.23-2.12 (m, 1H), 1.87-1.69 (m, 2H).

Step 5: A three-neck flask was charged with Pd-178 (7.44 mg, 15.6 μmol) and sodium tert-butoxide (22.5 mg, 234 μmol) and purged with N₂. A solution of (R)—N-(1-(3-chloro-2-fluoropyridin-4-yl)pent-4-en-1-yl)-4-methoxyaniline (I-1e) (50.0 mg, 156 μmol) in toluene (1 ml) was added dropwise. The resulting mixture was heated to 95° C. for 1.5 h. The reaction mixture was cooled to Rt and filtered through celite, washing with EtOAc (3×20 ml). The filtrate was concentrated in vacuo. The product was purified by silica gel chromatography (0%-50% EtOAc/heptane) to give (5R,8S)-1-fluoro-10-(4-methoxyphenyl)-6,7,8,9-tetrahydro-5H-5,8-epiminocyclohepta[c]pyridine (I-1f) a white solid. LCMS (method 1) m/z 285.3 (M+H)+(ES+), at 1.35 min. ¹H NMR (500 MHz, DMSO-d6) δ 7.95 (d, J=4.9 Hz, 1H), 7.29 (dd, J=5.0, 1.7 Hz, 1H), 6.80 (d, J=9.1 Hz, 2H), 6.71 (d, J=9.1 Hz, 2H), 4.92 (d, J=5.6 Hz, 1H), 4.56 (t, J=5.9 Hz, 1H), 3.61 (s, 3H), 2.92 (dd, J=17.6, 4.9 Hz, 1H), 2.35 (d, J=17.5 Hz, 1H), 2.32-2.19 (m, 2H), 1.91-1.82 (m, 1H), 1.81-1.74 (m, 1H).

Step 6: A solution of (5R,8S)-1-fluoro-10-(4-methoxyphenyl)-6,7,8,9-tetrahydro-5H-5,8-epiminocyclohepta[c]pyridine (I-1f) (69 mg, 232 μmol) in MeCN (3.2 ml) was cooled to 0° C. before a solution of CAN (381 mg, 696 μmol) in water (3.2 ml) was added dropwise. After the addition was completed the reaction was stirred for 1 h at 0° C. 2 M aqueous sodium hydroxide (5 ml) and water (5 ml) were added and the mixture extracted with DCM (3×10 ml). The combined organic layers were dried over magnesium sulfate and concentrated in vacuo to give (5R,8S)-1-fluoro-6,7,8,9-tetrahydro-5H-5,8-epiminocyclohepta[c]pyridine (I-1) as a pale pink solid. LCMS (method 1): m/z 179.2 (M+H)+(ES+); at 0.69 min, ¹H NMR (500 MHz, DMSO-d6) δ 7.91 (dd, J=5.0, 0.9 Hz, 1H), 7.04 (dd, J=5.0, 1.9 Hz, 1H), 4.17 (d, J=5.3 Hz, 1H), 3.78 (t, J=6.0 Hz, 1H), 2.85 (dd, J=17.1, 5.2 Hz, 1H), 2.74 (s, 1H), 2.38 (d, J=17.0 Hz, 1H), 2.01-1.85 (m, 2H), 1.73 (t, J=9.1 Hz, 1H), 1.51 (dt, J=9.4, 5.4 Hz, 1H).

### Intermediate 2 (I-2) Route 1

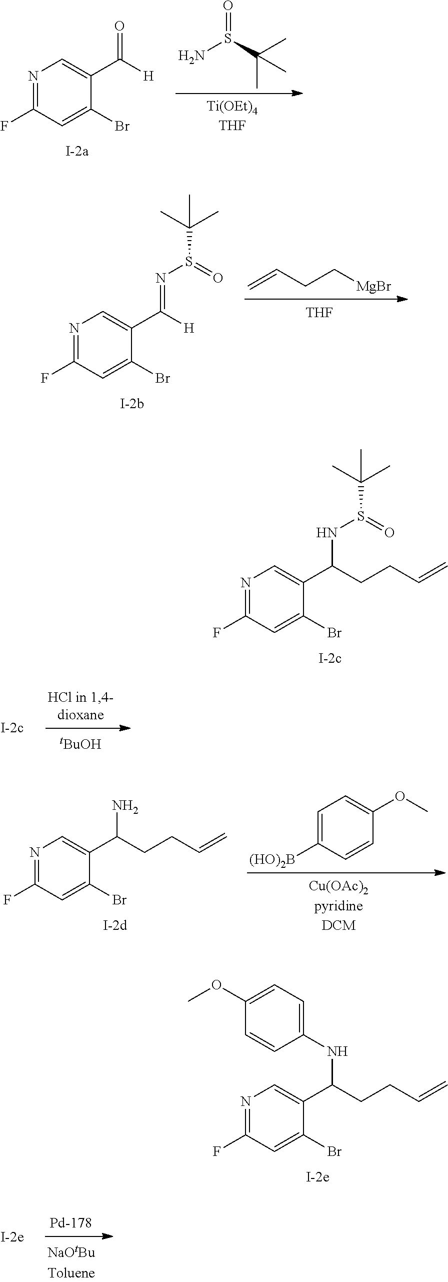

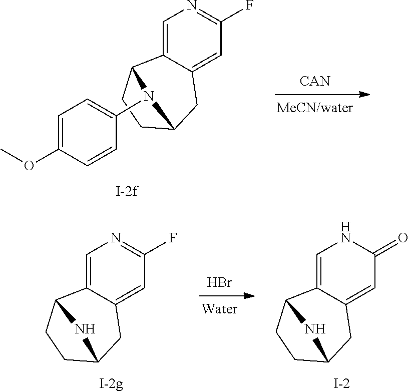

Step 1: (S,E)-N-((4-bromo-6-fluoropyridin-3-yl)methylene)-2-methylpropane-2-sulfinamide I-2b was synthesised from 4-bromo-6-fluoronicotinaldehyde I-2a using a procedure essentially the same as for I-1b. LCMS (method 2): m/z 307.0, 308.9 (M+H)+(ES+); at 2.05 min, ¹H NMR (500 MHz, DMSO-d6) δ 8.81 (s, 1H), 8.73 (s, 1H), 7.88 (d, J=2.4 Hz, 1H), 1.21 (s, 9H).

Step 2: To a solution of ((S,E)-N-((4-bromo-6-fluoropyridin-3-yl)methylene)-2-methylpropane-2-sulfinamide I-2b (7.00 g, 22.8 mmol) in THF (114 ml) was added but-3-en-1-yl magnesium bromide (2.54 M, 17.9 ml, 45.6 mmol) dropwise at −78° C. The mixture was warmed to RT slowly and stirred for 16 h. Saturated ammonium chloride solution (10 ml) was added and the product was extracted with EtOAc (2×10 ml). The combined organics were dried with magnesium sulfate and concentrated in vacuo. The product was purified by silica gel chromatography (0-10% IPA/isohexane) to give a 2:1 mixture of the diastereomers of (S)—N-(1-(4-bromo-6-fluoropyridin-3-yl)pent-4-en-1-yl)-2-methylpropane-2-sulfinamide I-2c as a pale yellow oil. LCMS (method 1) m/z 363.3, 365.4 (M+H)+(ES+), at 1.39 and 1.42 min.

Major diastereomer: ¹H NMR (500 MHz, DMSO-d6) δ 8.43 (s, 1H), 7.60 (d, J=2.6 Hz, 1H), 6.00 (d, J=9.5 Hz, 1H), 5.82 (ddt, J=16.7, 10.3, 6.6 Hz, 1H), 5.12-4.97 (m, 2H), 4.64-4.50 (m, 1H), 2.26-2.14 (m, 1H), 2.16-2.05 (m, 1H), 1.94-1.78 (m, 1H), 1.78-1.66 (m, 1H), 1.13 (s, 9H).

Minor diastereomer: ¹H NMR (500 MHz, DMSO-d6) δ 8.33 (s, 1H), 7.61 (d, J=2.5 Hz, 1H), 5.82 (ddt, J=16.7, 10.3, 6.6 Hz, 1H), 5.76 (d, J=7.0 Hz, 1H), 5.12-4.97 (m, 2H), 4.64-4.50 (m, 1H), 2.26-2.14 (m, 1H), 2.16-2.05 (m, 1H), 2.03-1.92 (m, 1H), 1.94-1.78 (m, 1H), 1.07 (s, 9H).

Step 3: 1-(4-Bromo-6-fluoropyridin-3-yl)pent-4-en-1-amine I-2d was synthesised from (S)—N-(1-(4-bromo-6-fluoropyridin-3-yl)pent-4-en-1-yl)-2-methylpropane-2-sulfinamide I-2c using a procedure essentially the same as for I-1d. LCMS (method 1) m/z 259.2, 261.2 (M+H)+(ES+), at 1.21 min. ¹H NMR (500 MHz, DMSO-d6) δ 8.41 (s, 1H), 7.55 (d, J=2.7 Hz, 1H), 5.82 (ddt, J=16.9, 10.2, 6.6 Hz, 1H), 5.03 (dq, J=17.2, 1.8 Hz, 1H), 4.95 (ddt, J=10.2, 2.3, 1.3 Hz, 1H), 4.12 (dd, J=8.2, 4.9 Hz, 1H), 2.25-1.98 (m, 4H), 1.75-1.64 (m, 1H), 1.64-1.54 (m, 1H).

Step 4: N-(1-(4-Bromo-6-fluoropyridin-3-yl)pent-4-en-1-yl)-4-methoxyaniline I-2e was synthesised from 1-(4-bromo-6-fluoropyridin-3-yl)pent-4-en-1-amine I-2d using a procedure essentially the same as for I-1e. LCMS (method 1) m/z 365.0, 367.1 (M+H)+(ES+), at 1.76 min. ¹H NMR (500 MHz, DMSO-d6) δ 8.21 (s, 1H), 7.61 (d, J=2.5 Hz, 1H), 6.66 (d, J=8.9 Hz, 2H), 6.40 (d, J=9.0 Hz, 2H), 6.04 (d, J=8.3 Hz, 1H), 5.86 (ddt, J=17.0, 10.2, 6.6 Hz, 1H), 5.08-4.90 (m, 2H), 4.59 (td, J=8.5, 4.8 Hz, 1H), 3.58 (s, 3H), 2.35-2.26 (m, 1H), 2.23-2.13 (m, 1H), 1.92-1.71 (m, 2H).

Step 5: A three-neck flask was charged with Pd-178 (0.29 g, 0.61 mmol) and NaO^{t}Bu (0.88 g, 9.2 mmol) and purged with N₂. A solution of N-(1-(4-bromo-6-fluoropyridin-3-yl)pent-4-en-1-yl)-4-methoxyaniline I-2e (2.3 g, 6.1 mmol) in toluene (66 ml) was added dropwise. The resulting mixture was heated to 95° C. for 2 h. The reaction mixture was cooled to RT and filtered through celite, washing the solid with EtOAc (150 ml). The filtrate was concentrated in vacuo. The product was purified by silica gel chromatography (0%-50% EtOAc/isohexane) to give a mixture of enantiomers which were dissolved to 30 mg/ml in DCM:methanol (1:4) and was then separated by chiral SFC on a Waters prep 15 with UV detection by at 210 nm, 40° C., 100 bar on a Lux C3 column (21.2 mm×250 mm, 5 μm particle size), flow rate 50 ml/min⁻¹ using 40% methanol to give (6S,9R)-3-fluoro-10-(4-methoxyphenyl)-6,7,8,9-tetrahydro-5H-6,9-epiminocyclohepta[c]pyridine I-2f as a pale yellow oil. LCMS (method 1) m/z 285.3 (M+H)+(ES+), at 1.35 min. ¹H NMR (500 MHz, DMSO-d6) δ 8.10 (s, 1H), 6.79 (t, J=9.7 Hz, 3H), 6.70 (d, J=9.1 Hz, 2H), 4.95 (d, J=5.5 Hz, 1H), 4.47 (t, J=6.0 Hz, 1H), 3.61 (s, 3H), 3.11 (dd, J=18.2, 4.9 Hz, 1H), 2.31-2.19 (m, 2H), 1.88-1.55 (m, 2H), 1H obscured by residual DMSO peak.

Step 6: (6S,9R)-3-fluoro-6,7,8,9-tetrahydro-5H-6,9-epiminocyclohepta[c]pyridine I-2g was synthesised from (6S,9R)-3-fluoro-10-(4-methoxyphenyl)-6,7,8,9-tetrahydro-5H-6,9-epiminocyclohepta[c]pyridine I-2f using a procedure essentially the same as for I-1. LCMS (method 1) m/z 179.2 (M+H)+(ES+), at 0.71 min.

Step 7: (6S,9R)-3-fluoro-6,7,8,9-tetrahydro-5H-6,9-epiminocyclohepta[c]pyridine I-2g (300 mg, 1.68 mmol) was dissolved in HBr (48% w/w in water, 1.90 ml, 16.8 mmol) and was heated at 100° C. for 18 h. The reaction mixture was cooled to RT, diluted with MeOH, loaded onto a SCX cartridge (20 g) and the product was eluted with NH₃ in MeOH solution (0.7M) to give (6S,9R)-2,5,6,7,8,9-hexahydro-3H-6,9-epiminocyclohepta[c]pyridin-3-one I-2 as an off white solid. LCMS (method 1) m/z 176.9 (M+H)+(ES+), at 0.41 min

### Intermediate 2 (I-2)-route 2

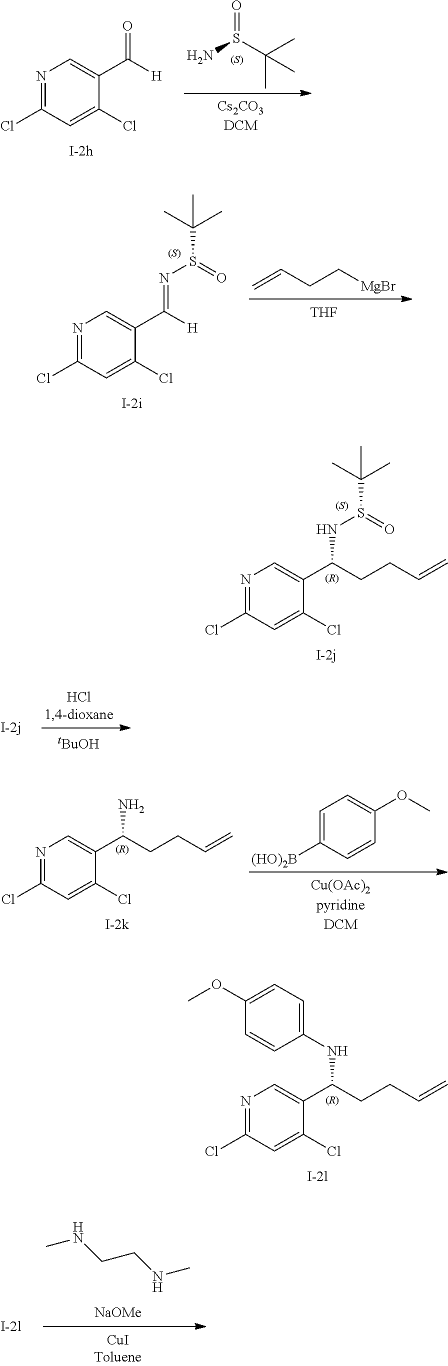

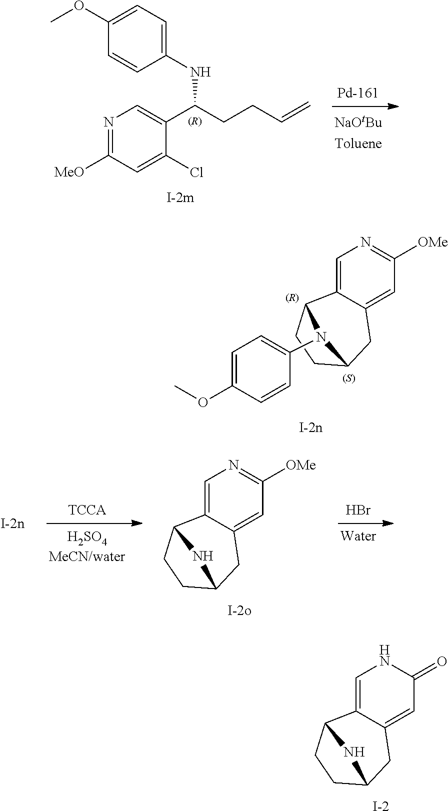

Step 1: To a solution of 4,6-dichloronicotinaldehyde I-2h (17.3 g, 88.5 mmol) and (S)-2-methylpropane-2-sulfinamide (10.7 g, 88.5 mmol) in DCM (150 ml) was added cesium carbonate (28.8 g, 88.5 mmol). The resultant mixture was stirred at RT for 16 h. The material was filtered and the solid residue was washed with DCM (150 ml). The solvent was evaporated to give (S)—N-((4,6-dichloropyridin-3-yl)methylene)-2-methylpropane-2-sulfinamide I-2i as an off white solid. ¹H NMR (400 MHz, DMSO-d6) δ 8.97 (s, 1H), 8.76 (s, 1H), 8.04 (s, 1H), 1.21 (s, 9H).

Step 2: A solution of but-3-en-1-ylmagnesium bromide (0.5 M in THF) (60.9 ml, 30.4 mmol) was slowly added to a solution of (S)—N-((4,6-dichloropyridin-3-yl)methylene)-2-methylpropane-2-sulfinamide I-2i (5.00 g, 1 Eq, 17.9 mmol) in THF (100 mL) at −78° C. The reaction mixture was allowed to slowly warm up to RT over 16 h. The reaction was cooled to 0-10° C. and saturated aqueous ammonium chloride (100 ml) was added and stirred for 5 minutes. The layers were separated and the aqueous phase was extracted with EtOAc (2×250 ml). The combined organics were washed with brine (50 ml), dried with Na₂SO₄ and concentrated in vacuo. The product was purified by chromatography on silica gel (0-30% MTBE in DCM) to afford (S)—N—((R)-1-(4,6-dichloropyridin-3-yl)pent-4-en-1-yl)-2-methylpropane-2-sulfinamide I-2j as a clear yellow oil. ¹H NMR (400 MHz, DMSO-d6) δ 8.53 (s, 1H), 7.77 (s, 1H), 5.85-5.75 (m, 2H), 5.07 (dq, J=17.2, 1.6 Hz, 1H), 5.00 (ddt, J=10.3, 2.3, 1.3 Hz, 1H), 4.66-4.56 (m, 1H), 2.20-2.05 (m, 2H), 1.98 (dtd, J=13.9, 8.0, 5.7 Hz, 1H), 1.83 (ddt, J=13.3, 8.7, 6.5 Hz, 1H), 1.07 (s, 9H).

Step 3: A solution of HCl (4M in dioxane) (41 ml, 0.16 mol) was added to a solution of (S)—N—((R)-1-(4,6-dichloropyridin-3-yl)pent-4-en-1-yl)-2-methylpropane-2-sulfinamide I-2j (11 g, 33 mmol) in ^{t}BuOH (50 ml) and the reaction mixture was stirred at RT for 90 min. The reaction mixture was cooled in an ice bath and water (220 ml) was added and stirred for 10 min. The aqueous was extracted with MTBE (3×30 ml). The organic layer was extracted with water (2×30 ml). The aqueous was basified using saturated aqueous NaHCO₃ solution and more solid NaHCO₃ and the mixture was stirred for 15 min. The product was extracted with MTBE (3×100 ml). The combined organics were washed with water (500 ml), dried with Na₂SO₄ and concentrated in vacuo to to give (R)-1-(4,6-dichloropyridin-3-yl)pent-4-en-1-amine I-2k as an orange oil. ¹H NMR (400 MHz, DMSO-d6) δ 8.60 (s, 1H), 7.69 (s, 1H), 5.80 (ddt, J=16.9, 10.1, 6.6 Hz, 1H), 5.01 (dq, J=17.2, 1.7 Hz, 1H), 4.94 (ddt, J=10.2, 2.3, 1.3 Hz, 1H), 4.15 (dd, J=8.0, 5.2 Hz, 1H), 2.17-2.01 (m, 4H), 1.74-1.54 (m, 2H).

Step 4: To a solution of (R)-1-(4,6-dichloropyridin-3-yl)pent-4-en-1-amine I-2k (7.04 g, 30.5 mmol) in DCM (70 ml) was added (4-methoxyphenyl)boronic acid (13.9 g, 91.4 mmol), copper (II) acetate (6.09 g, 33.5 mmol) and Et₃N (21.2 ml, 152 mmol). The resultant mixture was stirred at RT for 20 h. A further portion of copper (II) acetate (2.21 g, 12.2 mmol), (4-methoxyphenyl)boronic acid (5.55 g, 36.6 mmol) and Et₃N (5.09 ml, 36.6 mmol) was added and the mixture was stirred for a further 20 h. 1 M HCl (200 ml) was added and the layers were separated. Ammonium hydroxide solution (28% w/v, 100 and 200 ml) was added to the organic and aqueous layers respectively. The layers were separated and the aqueous was extracted with DCM (100 ml). The combined organics were washed with water (100 ml) and dried with MgSO₄. The product was purified by chromatography on silica gel (0-50% EtOAc/isohexane) to afford I-N-(1-(4,6-dichloropyridin-3-yl)pent-4-en-1-yl)-4-methoxyaniline I-2l as a thick colourless oil. ¹H NMR (400 MHz, DMSO) δ 8.42 (s, 1H), 7.75 (s, 1H), 6.70-6.59 (m, 2H), 6.46-6.34 (m, 2H), 6.01 (d, J=8.5 Hz, 1H), 5.90-5.77 (m, 1H), 5.08-4.94 (m, 2H), 4.64 (td, J=8.6, 4.9 Hz, 1H), 3.58 (s, 3H), 2.27 (ddd, J=12.7, 9.3, 6.1 Hz, 1H), 2.22-2.08 (m, 1H), 1.92-1.72 (m, 2H).

Step 5: To a solution of I-N-(1-(4,6-dichloropyridin-3-yl)pent-4-en-1-yl)-4-methoxyaniline I-2l (2.73 g, 8.10 mmol) in toluene (20 ml) was added N,N-dimethylethane-1,2-diamine (86.8 μL, 810 μmol), copper(I) iodide (30.8 mg, 162 μmol) and sodium methoxide (656 mg, 1.5 Eq). The resultant mixture was heated at 100° C. for 96 h. The reaction mixture was filtered through a pad of celite and the filtrate was concentrated in vacuo. The product was purified by chromatography on silica gel (80 g cartridge, 0-30% MTBE in isohexane) to afford I-N-(1-(4-chloro-6-methoxypyridin-3-yl)pent-4-en-1-yl)-4-methoxyaniline I-2m as a thick yellow oil. ¹H NMR (400 MHz, DMSO) δ 8.17 (s, 1H), 6.94 (s, 1H), 6.66-6.62 (m, 2H), 6.43-6.37 (m, 2H), 5.91 (d, J=8.5 Hz, 1H), 5.89-5.78 (m, 1H), 5.07-4.93 (m, 2H), 4.57 (td, J=8.5, 5.1 Hz, 1H), 3.79 (s, 3H), 3.58 (s, 3H), 2.27 (dt, J=14.3, 7.3 Hz, 1H), 2.22-2.07 (m, 1H), 1.80 (dddd, J=20.8, 13.8, 10.1, 5.1 Hz, 2H).

Step 6: Pd-161 (763.6 mg, 1.65 mmol) and sodium 2-methylpropan-2-olate (2.38 g, 24.8 mmol) were placed in a 3-necked RB flask, which was purged under vacuum and backfilled with N₂ (3 times). N-(1-(4-Chloro-6-methoxypyridin-3-yl)pent-4-en-1-yl)-4-methoxyaniline I-2m (5.79 g, 16.52 mmol) was purged under vacuum and backfilled with N₂ (3 times). Toluene (180 ml) was added to amine and the resultant solution was transferred to the 3-necked RB flask. The RB flask was purged under vacuum and backfilled with N₂ (3 times). The resultant mixture was heated at 95° C. for 2 h. The reaction was cooled and filtered through a pad of celite. The filter cake was washed with EtOAc. The filtrate was concentrated in vacuo. The product was purified by chromatography on silica gel (0-50% EtOAc/isohexane) to afford (6S,9R)-3-methoxy-10-(4-methoxyphenyl)-6,7,8,9-tetrahydro-5H-6,9-epiminocyclohepta[c]pyridine I-2n as a light orange solid. ¹H NMR (500 MHz, DMSO-ds) δ 8.01 (s, 1H), 6.82-6.73 (m, 2H), 6.73-6.64 (m, 2H), 6.39 (s, 1H), 4.83 (d, J=5.5 Hz, 1H), 4.43 (m, 1H), 3.74 (s, 3H), 3.61 (s, 3H), 3.04 (dd, J=18.0, 4.9 Hz, 1H), 2.41 (d, J=17.9 Hz, 1H), 2.32-2.14 (m, 2H), 1.84-1.63 (m, 2H).

Step 7: To a solution of (6S,9R)-3-methoxy-10-(4-methoxyphenyl)-6,7,8,9-tetrahydro-5H-6,9-epiminocyclohepta[c]pyridine I-2n (2.00 g, 6.61 mmol) in MeCN (75 ml) and water (75 m) was added sulfuric acid (6.613 ml, 1 M, 6.61 mmol) was added followed by trichloroisocyanuric acid (769 mg, 3.31 mmol). The reaction mixture was stirred at RT for 16 h. The mixture was extracted with DCM (3×200 ml). The combined organics were extracted with water (50 ml). The aqueous layer was basified with KOH (5 M, 3.6 ml) and extracted with 10% MeOH in DCM (300 ml). More KOH (5 M, 1.8 ml) was added and the aqueous layer was extracted with 10% MeOH in DCM (100 ml). A further portion of KOH (5 M, 1.8 ml) was added and the aqueous layer was extracted with 10% MeOH in DCM (150 ml). The combined organics were dried over Na₂SO₄ and concentrated in vacuo to give (6S,9R)-3-methoxy-6,7,8,9-tetrahydro-5H-6,9-epiminocyclohepta[c]pyridine I-2o as a brown oil. ¹H NMR (400 MHz, DMSO) δ 7.78 (s, 1H), 6.47 (s, 1H), 4.17-4.11 (m, 1H), 3.76 (s, 3H), 3.66 (td, J=5.2, 2.7 Hz, 1H), 2.95 (ddd, J=17.4, 3.9, 2.4 Hz, 1H), 2.57 (s, 1H), 2.46 (dt, J=17.3, 1.2 Hz, 1H), 1.96-1.81 (m, 2H), 1.72-1.60 (m, 1H), 1.50-1.36 (m, 1H).

Step 8: A solution of (6S,9R)-3-methoxy-6,7,8,9-tetrahydro-5H-6,9-epiminocyclohepta[c]pyridine I-2o (0.99 g, 5.2 mmol) in HBr (48% in water) (8.8 ml, 78 mmol) was heated at reflux for 16 h. The mixture was concentrated in vacuo and the concentrate was diluted with MeOH, loaded onto a SCX cartridge (80 g), the cartridge was washed with MeOH and the product was eluted with NH₃ in MeOH solution (0.7M). To give (6S,9R)-2,5,6,7,8,9-hexahydro-3H-6,9-epiminocyclohepta[c]pyridin-3-one I-2 as a brown solid. ¹H NMR (400 MHz, DMSO) δ 11.11 (s, 1H), 7.03 (s, 1H), 6.01 (s, 1H), 4.04 (d, J=5.4 Hz, 1H), 3.62 (t, J=5.9 Hz, 1H), 2.83 (dd, J=17.7, 5.1 Hz, 1H), 2.40 (dt, J=17.7, 1.3 Hz, 1H), 1.92-1.78 (m, 2H), 1.68-1.56 (m, 1H), 1.45 (dt, J=9.3, 4.8 Hz, 1H), Exchangeable NH not observed

### Experimental Scheme 2

### Compound 5 (±)-N-(3,4-dichlorophenyl)-6,7,8,9-tetrahydro-5H-5,8-epiminocyclo-hepta[d]pyrimidine-10-carbothioamide

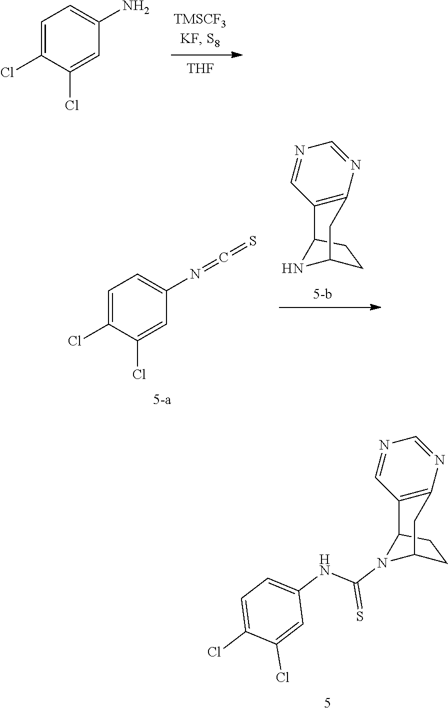 [SMILES: Nc1ccc(Cl)c(Cl)c1 ; S=C=Nc1ccc(Cl)c(Cl)c1 ; c1ncc2c(n1)CC1CCC2N1 ; S=C(Nc1ccc(Cl)c(Cl)c1)N1C2CCC1c1cncnc1C2 ; "THF"]

To a solution of elemental sulfur (127 mg, 494 μmol) and potassium fluoride (22 mg, 370 μmol) in THF (1 ml), under a nitrogen atmosphere was added trimethyl(trifluoromethyl)silane (146 μl, 988 μmol) was added, followed by a solution of 3,4-dichloroaniline (20.0 mg, 123 μmol) in THF (1 ml). The mixture was stirred at RT for 1 h. A solution of (5R,8S)-6,7,8,9-tetrahydro-5H-5,8-epiminocyclohepta[d]pyrimidine 5-b (22 mg, 136 μmol) in THF (0.5 ml) was added and the reaction mixture was stirred at RT for 16 h. The reaction mixture was diluted with 10% MeOH in DCM (5 ml) and poured into a saturated sodium bicarbonate aqueous solution (10 ml). The layers were separated and the aqueous layer was extracted with 10% MeOH in DCM (2×10 ml). The organic layers were combined, dried over magnesium sulfate and concentrated in vacuo. The product was purified by chromatography on silica gel (0-10% MeOH/DCM) to afford (±)-N-(3,4-dichlorophenyl)-6,7,8,9-tetrahydro-5H-5,8-epiminocyclohepta[d]pyrimidine-10-carbothioamide 5 as a light yellow solid. LCMS (method 1) m/z 365.1, 367.1 (M+H)+(ES+), at 1.28 min. ¹H NMR (400 MHz, DMSO-d6) δ 9.64 (s, 1H), 8.98 (s, 1H), 8.59 (s, 1H), 7.90-7.05 (m, 3H), 5.83 (s, 1H), 5.21 (s, 1H), 3.47 (s, 1H), 2.82 (d, J=18.1 Hz, 1H), 2.38-2.12 (m, 2H), 1.98-1.72 (m, 2H).

### Experimental Scheme 3

### Compound 6 (5R,8S,)—N′-Cyano-N-(3,4-dichlorophenyl)-1-fluoro-6,7,8,9-tetrahydro-5H-5,8-epiminocyclohepta[c]pyridine-10-carboximidamide

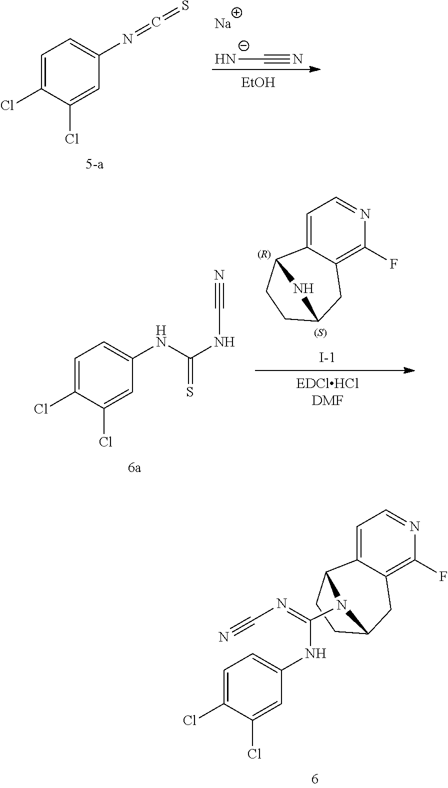

A solution of sodium hydrogencyanamide (35 mg, 539.0 μmol) and 1,2-dichloro-4-isothiocyanatobenzene (100 mg, 490.0 μmol) in EtOH (1 ml) was refluxed for 2 h. The reaction mixture was allowed to cool down to RT. EDCl·HCl (103 mg, 539.0 μmol) and (5R,8S)-1-fluoro-6,7,8,9-tetrahydro-5H-5,8-epiminocyclohepta[c]pyridine (107 mg, 509.6 μmol) and DMF (1 ml) were added, and the mixture was stirred at RT for 16 h. The reaction mixture was diluted with 10% MeOH in DCM (10 ml) and washed with water (3×10 ml) and brine (10 ml). The combined organics were dried over magnesium sulfate and concentrated in vacuo. The product was purified by chromatography on silica gel (0-100% EtOAc/isohexane) to afford (5R,8S)—N′-cyano-N-(3,4-dichlorophenyl)-1-fluoro-6,7,8,9-tetrahydro-5H-5,8-epiminocyclohepta[c]pyridine-10-carboximidamide as a pink solid. LCMS (method 1) m/z 390.2, 392.1 (M+H)+(ES+), at 1.27 min, ¹H NMR (500 MHz, DMSO-d6) δ 9.65 (s, 1H), 8.03 (d, J=5.0 Hz, 1H), 7.55 (d, J=8.7 Hz, 1H), 7.36 (d, J=2.5 Hz, 1H), 7.19 (s, 1H), 7.09 (dd, J=8.7, 2.6 Hz, 1H), 5.29 (s, 1H), 4.81 (s, 1H), 3.26-3.16 (m, 1H), 2.67 (d, J=17.4 Hz, 1H), 2.32-2.17 (m, 2H), 1.93-1.84 (m, 1H), 1.82-1.73 (m, 1H)

The following compounds were prepared using appropriate starting materials in an analogous procedure to that described in Experimental Scheme 3. Where the starting materials are not described in the literature, their synthesis is described below.

Key: ¹ purified by reverse phase Prep HPLC

| | | LCMS | |
|---|---|---|---|
| | | method 1 | |
| Compound | Structure | Rt [M + H]⁺ | NMR |
| 11¹ | 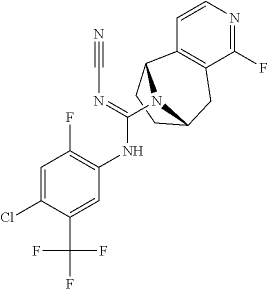 [SMILES: N#C/N=C(/Nc1cc(C(F)(F)F)c(Cl)cc1F)N1[C@H]2CC[C@@H]1c1ccnc(F)c1C2] | 442.1 at 1.12 min | ¹H NMR (400 MHz, DMSO-d6) δ 9.94 (s, 1H), 8.02 (d, J = 5.0 Hz, 1H), 7.78 (d, J = 10.9 Hz, 1H), 7.49 (d, J = 6.7 Hz, 1H), 7.19 (d, J = 5.0 Hz, 1H), 5.32 (d, J = 5.4 Hz, 1H), 4.85 (s, 1H), 3.29 − 3.20 (m, 1H), 2.65 (d, J = 17.1 Hz, 1H), 2.35 − 2.17 (m, 2H), 1.96 − 1.83 (m, 1H), 1.84 − 1.72 (m, 1H) |
| | (5R,8S)-N-(4-Chloro-2-fluoro-5- | | |
| | (trifluoromethyl)phenyl)-N′- | | |
| | cyano-1-fluoro-6,7,8,9- | | |
| | tetrahydro-5H-5,8-epimino- | | |
| | cyclohepta[c]pyridine-10- | | |
| | carboximidamide | | |
| 12¹ | 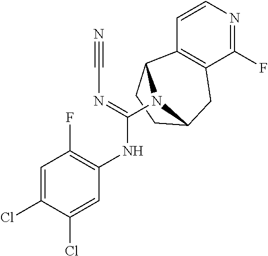 [SMILES: N#C/N=C(/Nc1cc(Cl)c(Cl)cc1F)N1[C@H]2CC[C@@H]1c1ccnc(F)c1C2] | 408.2 at 1.08 min | ¹H NMR (400 MHz, DMSO) δ 9.63 (s, 1H), 8.03 (d, J = 5.0 Hz, 1H), 7.71 (d, J = 10.1 Hz, 1H), 7.57 (d, J = 7.7 Hz, 1H), 7.22 − 7.15 (m, 1H), 5.32 (d, J = 5.7 Hz, 1H), 4.90 − 4.81 (m, 1H), 3.25 (dd, J = 17.4, 5.0 Hz, 1H), 2.65 (d, J = 17.4 Hz, 1H), 2.32 − 2.17 (m, 2H), 1.94 − 1.84 (m, 1H), 1.84 − 1.73 (m, 1H). |
| | (5R,8S)-N′-cyano-N-(4,5- | | |
| | dichloro-2-fluorophenyl)-1- | | |
| | fluoro-6,7,8,9-tetrahydro-5H- | | |
| | 5,8-epiminocyclohepta- | | |
| | [c]pyridine-10-carboximidamide | | |

### Experimental Scheme 4

### Compound 7: 2-(3,4-dichlorophenyl)-1-((5R,8S)-1-fluoro-6,7,8,9-tetrahydro-5H-5,8-epiminocyclohepta[c]pyridin-10-yl)-2-hydroxyethan-1-one

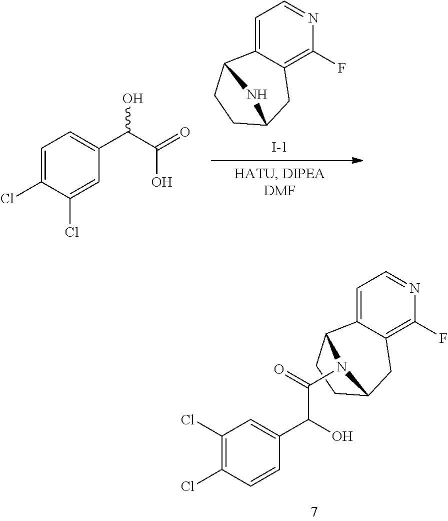

A solution of 2-(3,4-dichlorophenyl)-2-hydroxyacetic acid (25 mg, 112 μmol) and HATU (51 mg, 135 μmol) in DMF (0.75 ml) was stirred at RT for 15 min. A solution of (5R,8S)-1-fluoro-6,7,8,9-tetrahydro-5H-5,8-epiminocyclohepta[c]pyridine I-1 (20 mg, 112 μmol) and DIPEA (98 μL, 561 μmol) in DMF (0.75 ml) was added, and the reaction mixture was stirred at RT for 16 h. The reaction mixture was diluted with EtOAc (5 ml) and washed with brine (3×10 ml). The organic layer was dried over magnesium sulfate and concentrated in vacuo. The product was purified by prep-HPLC (25-55% MeCN/0.3% ammonia in water) to obtain 2-(3,4-dichlorophenyl)-1-((5R,8S)-1-fluoro-6,7,8,9-tetrahydro-5H-5,8-epiminocyclohepta[c]pyridin-10-yl)-2-hydroxyethan-1-one 7 as a colourless solid (Isomer 1; absolute stereochemistry unknown). LCMS (method 1) m/z 381.1, 383.1 (M+H)+(ES+) at 1.25 min. ¹H NMR (500 MHz, DMSO-d6) δ 7.90 (s, 1H), 7.50 (d, J=28.8 Hz, 2H), 7.30 (d, J=8.3 Hz, 1H), 7.00 (s, 1H), 6.07 (s, 1H), 5.44 (d, J=5.4 Hz, 1H), 5.35 (s, 1H), 4.86 (s, 1H), 2.55 (d, J=17.3 Hz, 1H), 2.15-2.00 (m, 2H), 1.84 (t, J=9.5 Hz, 1H), 1.68 (s, 1H), 1H obscured by residual water peak. NMR run at 90° C. as rotamers were observed at RT.

The following compounds were prepared using appropriate starting materials in an analogous procedure to that described in Experimental Scheme 4.

| | | LCMS | |
|---|---|---|---|
| | | method 1 | |
| Compound | Structure | Rt [M + H]⁺ | NMR |
| 8 | 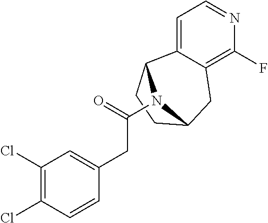 [SMILES: O=C(Cc1ccc(Cl)c(Cl)c1)N1[C@H]2CC[C@@H]1c1ccnc(F)c1C2] 2-(3,4-dichlorophenyl)-1- ((5R,8S)-1-fluoro-6,7,8,9- tetrahydro-5H-5,8-epimino- cyclohepta[c]pyridin-10- yl)ethan-1-one | 365.4, 367.4 at 1.39 min | 1H NMR (500 MHz, DMSO- d6) δ 8.00 (d, J = 5.0 Hz, 1H), 7.93 (d, J = 5.0 Hz, 1H), 7.54 (d, J = 8.3 Hz, 1H), 7.51 (d, J = 2.0 Hz, 1H), 7.42 (d, J = 8.3 Hz, 1H), 7.38 (d, J = 2.0 Hz, 1H), 7.28 − 7.19 (m, 2H), 7.12 (dd, J = 8.3, 2.1 Hz, 1H), 7.02 (dd, J = 5.0, 1.6 Hz, 1H), 5.76 − 5.07 (m, 2H), 4.82 (q, J = 6.1 Hz, 2H), 4.07 − 3.57 (m, 4H), 3.13 (dd, J = 17.4, 5.1 Hz, 1H), 2.99 (dd, J = 17.1, 5.0 Hz, 1H), 2.65 (d, J = 17.2 Hz, 1H), 2.55 (d, J = 17.1 Hz, 1H), 2.23 (ddd, J = 18.2, 12.4, 6.9 Hz, 2H), 2.18 − 2.02 (m, 2H), 1.92 − 1.74 (m, 3H), 1.66 (dt, J = 15.6, 8.3 Hz, 1H) NMR shows 1:1 mixture of |
| | | | rotamers |

### Experimental Scheme 5

### Compound 9: (5R,8S)-10-(4,5-dichloro-2-fluorobenzyl)-1-fluoro-6,7,8,9-tetrahydro-5H-5,8-epiminocyclohepta[c]pyridine

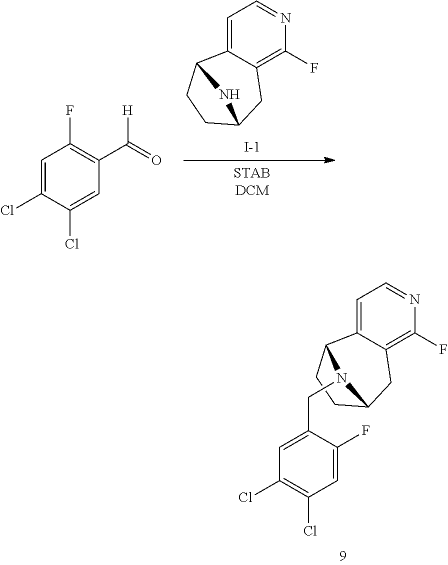 [SMILES: O=Cc1cc(Cl)c(Cl)cc1F ; Fc1nccc2c1C[C@@H]1CC[C@H]2N1 ; [I-] ; Fc1cc(Cl)c(Cl)cc1CN1[C@H]2CC[C@@H]1c1ccnc(F)c1C2]

To a solution of (5R,8S)-1-fluoro-6,7,8,9-tetrahydro-5H-5,8-epiminocyclohepta[c]pyridine I-1 (30 mg, 168 μmol) in DCM (2 ml) was added, 4,5-dichloro-2-fluorobenzaldehyde (33 mg, 168 μmol) and sodium triacetoxyborohydride (36 mg, 168 μmol). The reaction was stirred at RT for 3 days. The reaction mixture was diluted with DCM (5 ml) and water was added (5 ml). The organic layer was washed with brine (5 ml), dried over magnesium sulfate and concentrated in vacuo. The product was purified by product mass directed HPLC (50-80% MeCN/0.3% ammonia in water, C18) to afford (5R,8S)-10-(4,5-dichloro-2-fluorobenzyl)-1-fluoro-6,7,8,9-tetrahydro-5H-5,8-epiminocyclohepta[c]pyridine 9 as a colourless solid. LCMS (method 1) m/z 355.3, 357.3 (M+H)+(ES+) at 1.83 min. ¹H NMR (500 MHz, DMSO-d6) δ 7.97 (d, J=4.9 Hz, 1H), 7.68 (d, J=7.1 Hz, 1H), 7.64 (d, J=9.5 Hz, 1H), 7.06 (dd, J=5.0, 1.7 Hz, 1H), 4.00 (d, J=6.1 Hz, 1H), 3.69 (d, J=14.3 Hz, 1H), 3.63-3.46 (m, 2H), 2.99 (dd, J=17.7, 5.0 Hz, 1H), 2.38 (d, J=17.6 Hz, 1H), 2.20 (ddt, J=19.3, 12.5, 6.9 Hz, 2H), 1.79-1.65 (m, 1H), 1.65-1.47 (m, 1H)

### Experimental Scheme 6

### Compound 10: 3,4-dichlorophenyl (±)-6,7,8,9-tetrahydro-5H-5,8-epiminocyclohepta-[d]pyrimidine-10-carboxylate

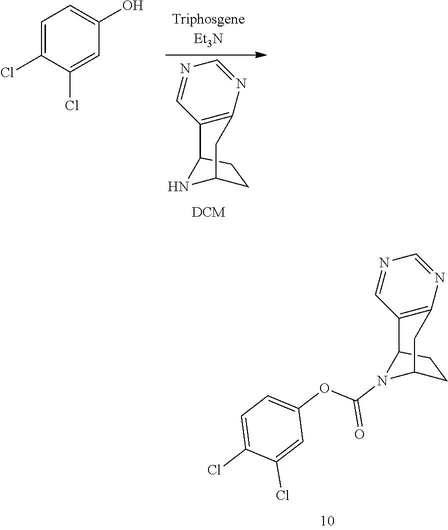 [SMILES: Oc1ccc(Cl)c(Cl)c1 ; c1ncc2c(n1)CC1CCC2N1 ; O=C(Oc1ccc(Cl)c(Cl)c1)N1C2CCC1c1cncnc1C2]

To a solution of triphosgene (26.5 mg, 89.3 μmol) in DCM (1 ml) and a solution of 3,4-dichlorophenol (35.6 mg, 218 μmol) in DCM 1 ml was added, followed by a slow addition of Et₃N (86 μl, 615 μmol) was added. The reaction mixture was stirred for 10 min at RT. This solution was added dropwise to a solution of (±)-6,7,8,9-tetrahydro-5H-5,8-epiminocyclohepta[d]pyrimidine (32.0 mg, 198 μmol) in DCM (2 ml) and the mixture was stirred at RT for 1 h. The reaction was diluted with 10% MeOH in DCM solution (8 ml) and saturated sodium bicarbonate solution (5 ml). The layers were separated and the aqueous was extracted with 10% MeOH in DCM solution (2×10 ml). The combined organic layers were passed through a hydrophobic frit and concentrated in vacuo. The product was purified by chromatography on silica gel (0-100% DCM/isohexane) to afford 3,4-dichlorophenyl (±)-6,7,8,9-tetrahydro-5H-5,8-epiminocyclohepta[d]pyrimidine-10-carboxylate as a colourless solid. LCMS (method 1) m/z 350.3, 352.3 (M+H)+(ES+) at 1.83 min. ¹H NMR (500 MHz, DMSO-d6) δ 9.00 (s, 1H), 8.66 (s, 1H), 7.63 (d, J=8.8 Hz, 1H), 7.55 (s, 1H), 7.19 (s, 1H), 5.22 (s, 1H), 4.75 (s, 1H), 3.41 (s, 1H), 2.85 (d, J=18.4 Hz, 1H), 2.32 (s, 2H), 1.95 (t, J=9.6 Hz, 1H), 1.80 (d, J=8.8 Hz, 1H).

### Experimental Scheme 7

### Compound 13: 1-(3,4-Dichlorophenyl)-2-((5R,8S)-1-fluoro-6,7,8,9-tetrahydro-5H-5,8-epiminocyclohepta[c]pyridin-10-yl)ethane-1,2-dione

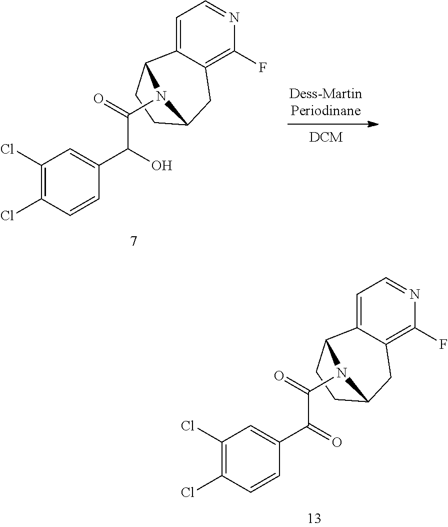 [SMILES: O=C(C(O)c1ccc(Cl)c(Cl)c1)N1[C@H]2CC[C@@H]1c1ccnc(F)c1C2 ; O=C(C(=O)N1[C@H]2CC[C@@H]1c1ccnc(F)c1C2)c1ccc(Cl)c(Cl)c1]

To a solution of 2-(3,4-dichlorophenyl)-1-((5R,8S)-1-fluoro-6,7,8,9-tetrahydro-5H-5,8-epiminocyclohepta[c]pyridin-10-yl)-2-hydroxyethan-1-one (0.043 g, 0.11 mmol) in DCM (5 ml) was added Dess-Martin periodinane (111 mg, 262 μmol) and the resultant mixture was stirred at RT for 16 h. A further portion of Dess-Martin periodinane (66 mg, 0.16 mmol) was added and the mixture was stirred at RT for 48 h. The reaction mixture was quenched with saturated sodium bicarbonate. The product was extracted with DCM (30 ml) and dried by passing through a hydrophobic frit. The solvent was removed in vacuo. The product was purified by chromatography on silica gel (0-50% EtOAc/isohexane) to afford 1-(3,4-dichlorophenyl)-2-((5R,8S)-1-fluoro-6,7,8,9-tetrahydro-5H-5,8-epiminocyclohepta[c]pyridin-10-yl)ethane-1,2-dione 13 as a thick colourless oil. LCMS (method 3) m/z 379.2, 381.2 (M+H)+(ES+) at 1.96 min. ¹H NMR (400 MHz, DMSO) δ 8.15-8.06 (m, 1H), 8.01-7.97 (m, 1H), 7.92-7.84 (m, 1H), 7.84-7.76 (m, 1H), 7.36 (dd, J=4.9, 1.6 Hz, 1H), 7.07 (dd, J=5.1, 1.6 Hz, 1H), 5.56 (d, J=6.0 Hz, 0.5H), 5.04 (d, J=5.9 Hz, 1H), 4.52 (t, J=6.0 Hz, 0.5H), 3.14 (ddd, J=44.2, 17.4, 5.1 Hz, 1H), 2.74 (dd, J=28.8, 17.3 Hz, 1H), 2.23 (ddt, J=17.7, 11.8, 6.1 Hz, 2H), 2.01-1.85 (m, 1H), 1.85-1.73 (m, 1H). Rotamers observed.

### Experimental Scheme 8

### Compound 15: (5R,8S)—N-(4,5-dichloro-2-fluorophenyl)-1-fluoro-N′-hydroxy-6,7,8,9-tetrahydro-5H-5,8-epiminocyclohepta[c]pyridine-10-carboximidamide

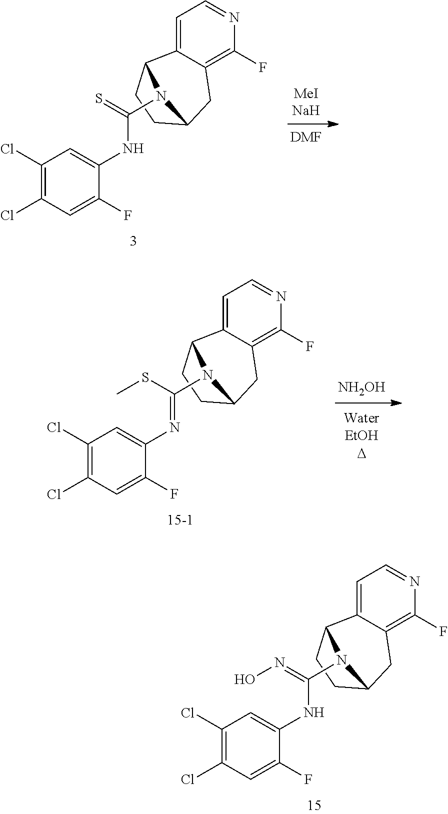

Step 1: To a solution of (5R,8S)—N-(4,5-dichloro-2-fluorophenyl)-1-fluoro-6,7,8,9-tetrahydro-5H-5,8-epiminocyclohepta[c]pyridine-10-carbothioamide 3 (500 mg, 1.15 mmol) in DMF (15 ml) was added sodium hydride (59.8 mg, 60% Wt, 1.49 mmol) and the mixture was stirred for 5 minutes. Iodomethane (101 μl, 1.61 mmol) was added and the mixture was stirred at RT for 2.5 h. Additional iodomethane (50 μl, 0.80 mmol) was added and the reaction mixture was stirred at RT for an additional 1.5 h. The mixture was diluted with MTBE (60 ml) and water (60 ml). The layers were separated, and the aqueous layer was further extracted with MTBE (2×60 ml). The combined organics were washed with brine, dried over MgSO₄ and concentrated in vacuo. The product was purified by chromatography on silica gel (0-100% DCM/isohexane) to afford methyl (5R,8S)—N-(4,5-dichloro-2-fluorophenyl)-1-fluoro-6,7,8,9-tetrahydro-5H-5,8-epiminocyclohepta[c]pyridine-10-carbimidothioate 15-1 as a thick colourless oil. LCMS (method 4) m/z 414.0, 416.0 (M+H)+(ES+) at 2.35 min. ¹H NMR (400 MHz, DMSO) δ 8.01 (d, J=5.0 Hz, 1H), 7.55 (d, J=10.0 Hz, 1H), 7.23 (dd, J=5.0, 1.6 Hz, 1H), 7.13 (d, J=8.0 Hz, 1H), 5.25 (app. d, J=5.3 Hz, 1H), 4.79-4.70 (m, 1H), 3.26 (dd, J=17.6, 5.2 Hz, 1H), 2.62 (d, J=17.3 Hz, 1H), 2.35-2.23 (m, 2H), 2.21 (s, 3H), 1.93-1.83 (m, 1H), 1.82-1.73 (m, 1H).

Step 2: To a solution of methyl (5R,8S)—N-(4,5-dichloro-2-fluorophenyl)-1-fluoro-6,7,8,9-tetrahydro-5H-5,8-epiminocyclohepta[c]pyridine-10-carbimidothioate 15-1 (50.0 mg, 05 μmol) in EtOH (1.0 ml) was added hydroxylamine in water (141 μl, 50% Wt, 2.30 mmol) and the mixture was stirred at 80° C. for 1.5 h. The solvent was removed in vacuo. The residue was redissolved in EtOH (1.0 ml) and stirred at 80° C. for 2 h. Additional hydroxylamine in water (150 μl, 50% Wt, 2.45 mmol) was added and the reaction was stirred at 80° C. for 3 days. The solvent was removed in vacuo. The product was purified by chromatography on silica gel (0-10% MeOH/DCM) to afford (5R,8S)—N′-(4,5-dichloro-2-fluorophenyl)-1-fluoro-N-hydroxy-6,7,8,9-tetrahydro-5H-5,8-epiminocyclohepta[c]pyridine-10-carboximidamide 15 as a yellow solid. LCMS (method 1) m/z 399.1, 401.1 (M+H)+(ES+) at 1.66 min. ¹H NMR (400 MHz, DMSO) δ 9.37 (s, 1H), 7.95 (d, J=5.0 Hz, 1H), 7.85 (s, 1H), 7.56 (d, J=10.8 Hz, 1H), 7.10 (dd, J=5.1, 1.6 Hz, 1H), 7.01 (d, J=8.1 Hz, 1H), 4.65 (d, J=5.9 Hz, 1H), 4.17 (t, J=6.0 Hz, 1H), 2.98 (dd, J=17.3, 5.0 Hz, 1H), 2.45 (d, J=17.3 Hz, 1H), 2.26-2.07 (m, 2H), 1.82-1.73 (m, 1H), 1.70-1.60 (m, 1H).

### Experimental Scheme 9

### Compound 16: (5R,8S)—N-(4,5-dichloro-2-fluorophenyl)-1-fluoro-N′-methoxy-6,7,8,9-tetrahydro-5H-5,8-epiminocyclohepta[c]pyridine-10-carboximidamide

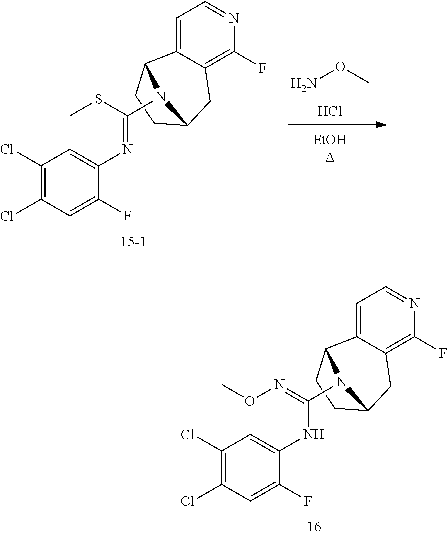

To a solution of methyl (5R,8S)—N-(4,5-dichloro-2-fluorophenyl)-1-fluoro-6,7,8,9-tetrahydro-5H-5,8-epiminocyclohepta[c]pyridine-10-carbimidothioate 15-1 (50 mg, 0.10 mmol) and pyridine (17 μL, 0.21 mmol) in EtOH (1.0 ml) was added O-methylhydroxylamine hydrochloride (8.8 mg, 0.10 mmol) and the resulting mixture was stirred at 50° C. for 5 h. The mixture was then stirred at 80° C. overnight. Saturated sodium bicarbonate solution (4 ml) was added and the product was extracted with EtOAc (2×4 ml). The combined organics were dried with magnesium sulfate and concentrated in vacuo. The product was purified by chromatography on silica gel (0-50% EtOAc/isohexane) followed by reverse phase Prep HPLC purification (55-100% MeCN/0.3% Ammonia in water) to give (5R,8S)—N-(4,5-dichloro-2-fluorophenyl)-1-fluoro-N′-methoxy-6,7,8,9-tetrahydro-5H-5,8-epiminocyclohepta[c]pyridine-10-carboximidamide 16 as a colourless solid. LCMS (method 1) m/z 412.4 (M+H)+(ES+) at 1.85 min. ¹H NMR (400 MHz, DMSO) δ 8.10 (s, 1H), 7.95 (d, J=5.0 Hz, 1H), 7.55 (d, J=10.7 Hz, 1H), 7.09 (dd, J=5.0, 1.6 Hz, 1H), 6.99 (d, J=8.0 Hz, 1H), 4.67 (d, J=5.7 Hz, 1H), 4.22 (t, J=6.0 Hz, 1H), 3.46 (s, 3H), 3.03 (dd, J=17.4, 5.1 Hz, 1H), 2.44 (d, J=17.3 Hz, 1H), 2.24-2.08 (m, 2H), 1.84-1.73 (m, 1H), 1.73-1.58 (m, 1H).

### Experimental Scheme 10

### Compound 17: (5R,8S)—N′-(4,5-dichloro-2-fluorophenyl)-1-fluoro-6,7,8,9-tetrahydro-5H-5,8-epiminocyclohepta[c]pyridine-10-carboximidamide

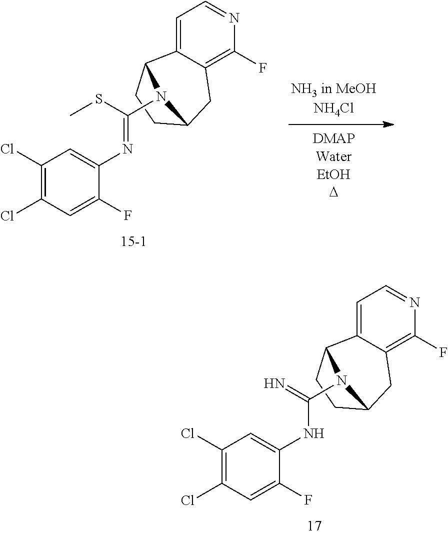 [SMILES: CS/C(=N\c1cc(Cl)c(Cl)cc1F)N1[C@H]2CC[C@@H]1c1ccnc(F)c1C2 ; "DMAPWaterEtOHD" ; "NH3 in MeOHNH4Cl" ; N=C(Nc1cc(Cl)c(Cl)cc1F)N1[C@H]2CC[C@@H]1c1ccnc(F)c1C2]

To a solution of methyl (5R,8S)—N-(4,5-dichloro-2-fluorophenyl)-1-fluoro-6,7,8,9-tetrahydro-5H-5,8-epiminocyclohepta[c]pyridine-10-carbimidothioate 15-1 (31 mg, 75 μmol) and DMAP (27 mg, 0.22 mmol) in EtOH (2 ml) was treated with ammonia in methanol (0.50 ml, 7 M, 3.5 mmol). The resulting mixture was stirred at 150° C. under microwave conditions for 30 min. The reaction mixture was treated with ammonium chloride (270 mg, 5.05 mmol) in water (2 ml) and the reaction mixture was stirred at 150° C. under microwave conditions for 1.5 h. The reaction mixture was treated with sat. ammonium chloride solution (10 ml) and extracted with EtOAc (3×10 ml). The combined organics were dried over sodium sulfate, filtered and concentrated in vacuo. The product was purified by chromatography on silica gel (0-100% EtOAc/isohexane) to afford (5R,8S)—N′-(4,5-dichloro-2-fluorophenyl)-1-fluoro-6,7,8,9-tetrahydro-5H-5,8-epiminocyclohepta[c]pyridine-10-carboximidamide 17 as a pale brown solid. LCMS (method 1) m/z 383.0, 385.0 (M+H)+(ES+) at 1.07 min. ¹H NMR (400 MHz, DMSO) δ 7.96 (d, J=5.0 Hz, 1H), 7.39 (d, J=10.1 Hz, 1H), 7.13-7.08 (m, 1H), 6.89 (d, J=8.1 Hz, 1H), 5.91 (s, 2H), 5.13 (d, J=5.1 Hz, 1H), 4.64 (s, 1H), 2.42 (d, J=17.1 Hz, 1H), 2.23 (d, J=7.0 Hz, 2H), 1.80 (t, J=9.4 Hz, 1H), 1.72 (d, J=9.6 Hz, 1H). (1H under water peak)

### Human GPR65 Cyclic Adenosine Monophosphate (cAMP) Homogeneous Time Resolved Fluorescence (HTRF) Antagonist Assay Procedure

IC₅₀ data was obtained by the following procedure:

1321N1 human astrocytoma cells stably expressing human recombinant GPR65 (1321N1-hrGPR65 cells, EuroscreenFast) were cultured according to the vendor's instructions.

Compounds were tested for their ability to antagonise GPR65, through measuring the concentration of cytoplasmic cAMP following treatment of the cells at a pH of 7.2 to activate GPR65 signalling and addition of the compound to be tested. The extent to which the expected rise in cAMP concentration upon GPR65 activation was suppressed by the added compound is indicative of its potency. The assay was carried out according to EuroscreenFast assay methodology as follows.

On the day of the assay, test compounds were added to 384-well, low volume, white microtiter plates by acoustic dispensing. KRH buffer (5 mM KCl, 1.25 mM MgSO₄, 124 mM NaCl, 25 mM HEPES, 13.3 mM Glucose, 1.25 mM KH₂PO₄ and 1.45 mM CaCl₂) was adjusted to pH 6.5, pH 7.6 and pH 8.4 by adding NaOH. 1321N1-hGPR65 cells were rapidly thawed and diluted in KRH, pH 7.6 prior to centrifugation at 300×g for 5 min and resuspension in assay buffer (KRH, pH 7.6, supplemented with 1 mM 3-isobutyl-1-methylxanthine (IBMX) and 200 μM ethylenediaminetetraacetic acid (EDTA)). Cells were added to assay plates at a density of 2,000 cells per well in a volume of 5 μl. Assay plates were briefly centrifuged at 100×g and then incubated at room temperature for 30 min. Cells were stimulated by the addition of 5 μL KRH, pH 6.5, to achieve an assay pH of 7.2, while control wells received 5 μl KRH, pH 8.4 to achieve an assay pH of 7.9. Assay plates were briefly centrifuged at 100×g and then incubated at room temperature for 30 min.

Accumulation of cAMP was detected by cAMP HTRF kit (Cisbio). d2-labeled cAMP and cryptate-labeled anti-cAMP antibody in Lysis and Detection Buffer (Cisbio) were added to assay plates, and the plates were incubated at room temperature for 1 h. HTRF measurements were performed using a Pherastar FSX instrument. Acceptor and donor emission signals were measured at 665 nm and 620 nm, respectively, and HTRF ratios were calculated as signal_{665 nm}/signal_{620 nm}×10⁴. Data were normalised to high and low control values and fitted with 4-parameter logistic regression to determine hGPR65 IC50 values for the test compounds, which are shown in Table 1.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

TABLE 1
Activity of selected compounds according to the invention

| | |
|---|---|
| 1 | High |
| 2 | High |
| 3 | High |
| 4 | High |
| 5 | High |
| 6 | High |
| 7 | Low |
| 8 | Medium |
| 9 | Medium |
| 10 | Medium |
| 11 | High |
| 12 | High |
| 13 | Medium |
| 15 | High |
| 16 | High |
| 17 | Low |

High = IC50 <500 nM; Medium = IC50 >500 nM and <5 μM; Low >5 μM

### REFERENCES

Bohn, T. et al. (2018). Tumor immunoevasion via acidosis-dependent induction of regulatory tumor-associated macrophages. Nature Immunology, 1319-1326.
Damaghi, M. et al. (2013). pH Sensing and Regulation in Cancer. Frontiers in Physiology.
Gaublomme, J. et al. (2015). Single-Cell Genomics Unveils Critical Regulators of Th17 Cell Pathogenicity. Cell, 1400-1412.
Hernandez, J. (2018). GPR65, a critical regulator of Th17 cell pathogenicity, is regulated by the CRTC2/CREB pathway. The Journal of Immunology, 200 (Supplement).
Korn, T. et al. (2009). IL-17 and Th17 Cells. Annual Reviews in Immunology, 485-517.
Wang, J. et al. (2004). TDAG8 is a proton-sensing and psychosine-sensitive G-protein-coupled receptor. Journal of Biological Chemistry, 45626-45633.
Yoshida, N. et al. (2016). ICER is requisite for Th17 differentiation. Nature Communications, 12993.
Hardin, M. et al. (2014). The clinical and genetic features of COPD-asthma overlap syndrome. Eur Respir J. 2014 August; 44(2):341-50.
Kottyan, L. et al. (2009). Eosinophil viability is increased by acidic pH in a cAMP- and GPR65-dependent manner. Blood. 2009 Sep. 24; 114(13):2774-82.
Tsurumaki, H. et al (2015). Int J Mol Sci. Protective Role of Proton-Sensing TDAG8 in Lipopolysaccharide-Induced Acute Lung Injury. December 4; 16(12):28931-42.
Schultz and Wolfe (2011), Organic Letters, Intramolecular Alkene Carboamination Reactions for the Synthesis of Enantiomerically Enriched Tropane Derivatives, 13 (11), 2962-2965

1. A compound of formula (I), or a pharmaceutically acceptable salt or solvate thereof, 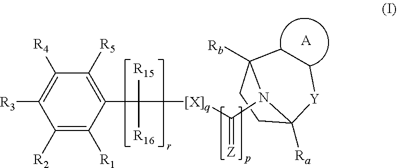
wherein:
ring A is a 5 or 6 membered aromatic or heteroaromatic ring, wherein said aromatic or heteroaromatic ring is optionally substituted with one or more substituents selected from halo, CN, alkoxy, NR₁₁R_{11′}, OH, CONR₁₉R₂₀, SO₂NR₁₉R₂₀, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R_{11′}, OH, alkyl, haloalkyl, and aralkyl;
Y is selected from CH₂, C═N—OH, and CR₁₀R_{10′};
Rₐ and R_{b} are each independently selected from H and alkyl;
Z is selected from O, S, NR₁₇ and CR₁₈;
X is selected from O and NH;
p is 0, 1 or 2;
q is 0 or 1; and
r is 0 or 1;
wherein at least one of p, q and r is other than zero, and with the proviso that:
(i) when r is 0, p is 1 and q is 1, when X is NH, Z is other than 0;
(ii) when r and q are both 0, and p is 1, Z is not O;
R₁, R₄, and R₅ are each independently selected from H, alkyl, alkoxy, OH, F, Cl, Br, and I;
R₂ and R₃ are each independently selected from H, F, Cl, Br, I, CN, methoxy, haloalkyl, haloalkoxy and CO₂-alkyl;
R₁₀ and R_{10′} are each independently selected from H, F, alkyl, and haloalkyl;
R₁₁ and R₁˜ are each independently selected from H, alkyl, haloalkyl, COR₁₂, CO₂R₁₂ and SO₂R₁₃;
R₁₂ and R₁₃ are each independently alkyl;
R₁₅ and R₁₆ are each independently selected from H, alkoxy, alkyl and OH;
R₁₇ is selected from H, CN, OH, alkoxy and alkyl;
R₁₈ is NO₂ or CN;
each R₁₉ is independently H or alkyl; and
each R₂₀ is independently alkyl;
and wherein the compound is other than:
(6S,9R)-10-benzyl-4-chloro-6,7,8,9-tetrahydro-5H-6,9-epiminocyclohepta[d]-pyrimidine;
(6S,9R)-10-benzyl-1,5,6,7,8,9-hexahydro-4H-6,9-epiminocyclohepta[d]pyrimidin-4-one;
(6R,9S)-10-benzyl-1,5,6,7,8,9-hexahydro-4H-6,9-epiminocyclohepta[d]pyrimidin-4-one;
10-benzyl-1,5,6,7,8,9-hexahydro-4H-6,9-epiminocyclohepta[d]pyrimidin-4-one;
10-(2,4,6-trimethoxybenzyl)-6,7,8,9-tetrahydro-5H-5,8-epiminocyclohepta[d]-pyrimidine; and
2-methoxy-5-((6,7,8,9-tetrahydro-5H-5,8-epiminocyclohepta[d]pyrimidin-10-yl)methyl)-benzonitrile.

2. A compound according to claim 1, wherein p is 1, q is 1 and r is 0.

3. A compound according to claim 2, wherein X is NH, and Z is selected from S and NR₁₇.

4. A compound according to claim 2 or claim 3, wherein X is NH, and Z is S.

5. A compound according to claim 2 or claim 3, wherein X is NH, and Z is NR₁₇, where R₁₇ is preferably CN.

6. A compound according to claim 2, wherein X is O and Z is O.

7. A compound according to claim 1, wherein p is 1, q is 0 and r is 1.

8. A compound according to claim 7, wherein Z is O and R₁₅ and R₁₆ are both H, or one of R₁₅ and R₁₆ is H and the other is selected from H and OH.

9. A compound according to claim 1, wherein p is 0, q is 0 and r is 1.

10. A compound according to claim 9, wherein R₁₅ and R₁₆ are both H.

11. A compound according to claim 1, wherein p is 2, q is 0 and r is 0.

12. A compound according to claim 11, wherein Z is O.

13. A compound according to any preceding claim, wherein ring A is selected from a pyridine moiety and a pyrimidine moiety, each of which may be optionally substituted.

14. A compound according to any one of claims 1 to 12, wherein ring A is selected from the groups (i)-(xix): 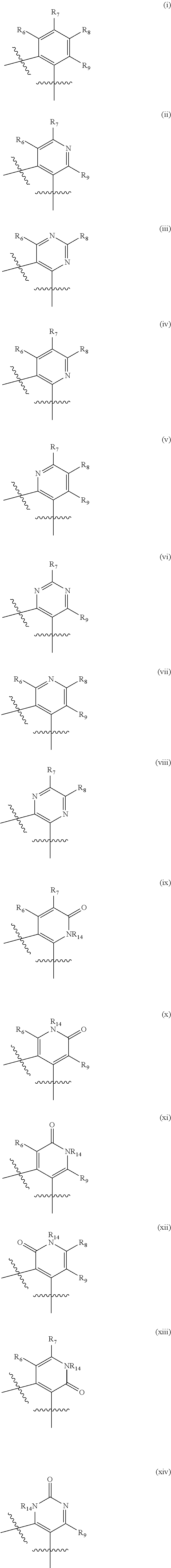 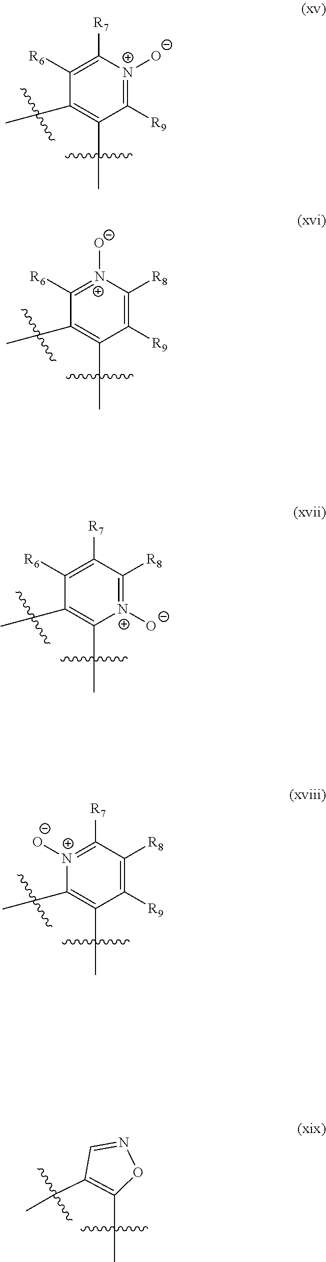
wherein R₆, R₇, R₈, and R₉ are each independently selected from H, F, Cl, Br, I, CN, alkoxy, NR₁₁R_{11′}, OH, alkyl, phenyl, and haloalkyl, and R₁₄ is H or alkyl, more preferably H.

15. A compound according to claim 14, wherein ring A is selected from (ii), (iii) and (x).

16. A compound according to any preceding claim, wherein Y is selected from CH₂ and C═N—OH, and is preferably CH₂.

17. A compound according to any preceding claim, wherein R₂ and R₃ are each independently selected from F, Cl, Br, I, CN, C₁-C₆ haloalkyl, C₁-C₆ haloalkoxy and CO₂-alkyl.

18. A compound according to any preceding claim, wherein R₂ and R₃ are each independently selected from Cl, Br, and CF₃, and more preferably are each independently selected from Cl and CF₃.

19. A compound according to any preceding claim, wherein R₂ and R₃ are both Cl, or one of R₂ and R₃ is CF₃ and the other is selected from Cl and Br.

20. A compound according to any preceding claim, wherein R₁ and R₄ are both H.

21. A compound according to any preceding claim, wherein R₅ is selected from H, F and CN, and is preferably H or F.

22. A compound according to any preceding claim, wherein:
R₁ is H;
R₂ is selected from Br and Cl;
R₃ is selected from CF₃ and Cl;
R₄ is H; and
R₅ is selected from H and F.

23. A compound according to claim 14, wherein R₆, R₇, R₈, and R₉ are each independently selected from H, F, C, Br, CN, OMe, NH₂, NHBu, NHCO₂Bu and OH, more preferably H and F.

24. A compound according to any preceding claim, which is of formula (I.1): 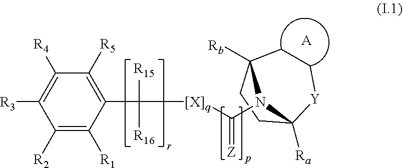
where A, X, Y, Z, p, q, r, Rₐ, R_{b}, R₁-R₅, R₁₅ and R₁₆ are as defined in any of claims 1-23.

25. A compound according to any of claims 1-23, which is in the form of a mixture that is enantiomerically enriched with a compound of formula (I.1): 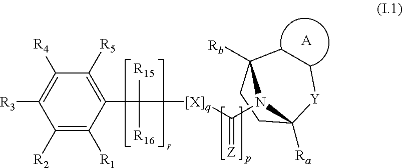
where A, X, Y, Z, p, q, r, Rₐ, R_{b}, R₁-R₅, R₁₅ and R₁₆ are as defined in any of claims 1-23.

26. A compound according to any preceding claim which is selected from the following: 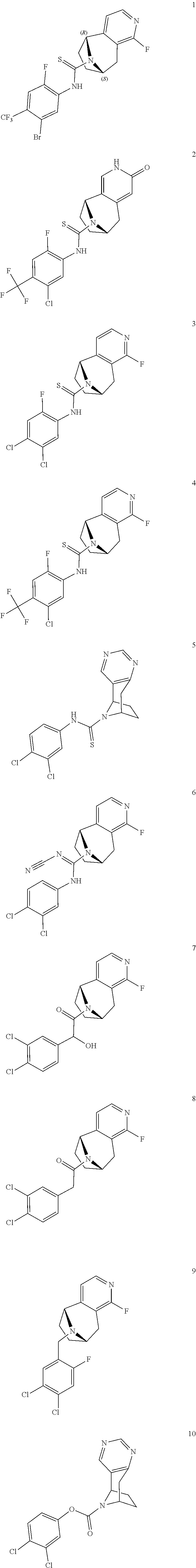 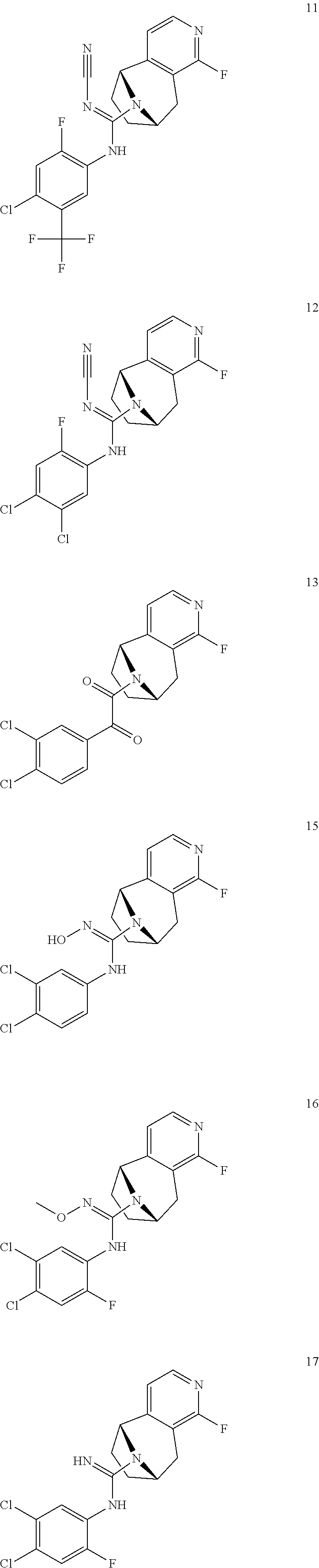 [SMILES: N#C/N=C(/Nc1cc(C(F)(F)F)c(Cl)cc1F)N1[C@H]2CC[C@@H]1c1ccnc(F)c1C2 ; N#C/N=C(/Nc1cc(Cl)c(Cl)cc1F)N1[C@H]2CC[C@@H]1c1ccnc(F)c1C2 ; O=C(C(=O)N1[C@H]2CC[C@@H]1c1ccnc(F)c1C2)c1ccc(Cl)c(Cl)c1 ; O/N=C(\Nc1ccc(Cl)c(Cl)c1)N1[C@H]2CC[C@@H]1c1ccnc(F)c1C2 ; CO/N=C(\Nc1cc(Cl)c(Cl)cc1F)N1[C@H]2CC[C@@H]1c1ccnc(F)c1C2 ; N=C(Nc1cc(Cl)c(Cl)cc1F)N1[C@H]2CC[C@@H]1c1ccnc(F)c1C2]
and enantiomers thereof, and mixtures of enantiomers thereof, including racemic mixtures, and pharmaceutically acceptable salts and solvates thereof.

27. A pharmaceutical composition comprising a compound according to any of claims 1-26, and a pharmaceutically acceptable diluent, excipient, or carrier.

28. A compound according to any one of claims 1-26, or a pharmaceutical composition according to claim 27, for use as a medicament.

29. A compound according to any one of claims 1-26, or a pharmaceutical composition according to claim 27, for use in treating or preventing a disorder selected from a proliferative disorder, an immune disorder, asthma, chronic obstructive pulmonary disease (COPD) and acute respiratory distress syndrome (ARDS).

30. A compound or pharmaceutical composition for use according to claim 29, wherein the use comprises modulating GPR65, preferably wherein the use comprises inhibiting GPR65 signalling.

31. A compound or pharmaceutical composition for use according to claim 29, wherein the disorder is a proliferative disorder.

32. A compound or pharmaceutical composition for use according to claim 31, wherein the proliferative disorder is a cancer, and is preferably a solid tumour and/or metastases thereof.

33. A compound or pharmaceutical composition for use according to claim 32, wherein the proliferative disorder is a cancer is selected from melanoma, renal cell carcinoma (RCC), gastric cancer, acute myeloid leukaemia (AML), triple negative breast cancer (TNBC), head and neck cancer, colorectal cancer, colorectal adenocarcinoma, pancreatic adenocarcinoma, sarcoma, lung cancer, ovarian cancer and gliomas, preferably glioblastoma (GBM).

34. A compound or pharmaceutical composition for use according to claim 29 wherein the disorder is an immune disorder.

35. A compound or pharmaceutical composition for use according to claim 34, wherein the immune disorder is an autoimmune disease.

36. A compound or pharmaceutical composition for use according to claim 35, wherein the autoimmune disease is selected from psoriasis, psoriatic arthritis, rheumatoid arthritis (RA), multiple sclerosis (MS), systemic lupus erythematosus (SLE), autoimmune thyroiditis (Hashimoto's thyroiditis), Graves' disease, uveitis (including intermediate uveitis), ulcerative colitis, Crohn's disease, autoimmune uveoretinitis, systemic vasculitis, polymyositis-dermatomyositis, systemic sclerosis (scleroderma), Sjogren's Syndrome, ankylosing spondylitis and related spondyloarthropathies, sarcoidosis, autoimmune hemolytic anemia, immunological platelet disorders, and autoimmune polyendocrinopathies.

37. A compound or pharmaceutical composition for use according to claim 36, wherein the autoimmune disease is selected from psoriasis, psoriatic arthritis, ankylosing spondylitis, Crohn's disease, and multiple sclerosis (MS).

38. A compound or pharmaceutical composition for use according to claim 29, wherein the use comprises treating or preventing a disorder selected from asthma, chronic obstructive pulmonary disease (COPD) and acute respiratory distress syndrome (ARDS).

39. A method of treating a disorder as defined in any of claims 29-38, comprising administering to a subject a compound as defined in any of claims 1-26, or a pharmaceutical composition as defined in claim 27.

40. A compound as defined in any one of claims 1-26, or a pharmaceutically acceptable salt or solvate thereof, for use in treating or preventing a GPR65-associated disease or disorder.

41. Use of a compound as defined in any one of claims 1-26, or a pharmaceutically acceptable salt or solvate thereof, in the preparation of a medicament for treating or preventing a GPR65-associated disease or disorder in a subject.

42. Use of a compound as defined in any one of claims 1-26, or a pharmaceutically acceptable salt or solvate thereof, in the preparation of a medicament for treating or preventing a disorder selected from a proliferative disorder, an immune disorder, asthma, chronic obstructive pulmonary disease (COPD) and acute respiratory distress syndrome (ARDS).

43. A compound of formula (Ih), or a pharmaceutically acceptable salt or solvate thereof, 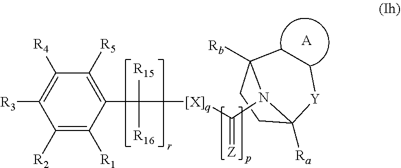
wherein:
ring A is a 5 or 6 membered aromatic or heteroaromatic ring, wherein said aromatic or heteroaromatic ring is optionally substituted with one or more substituents selected from halo, CN, alkoxy, NR₁₁R_{11′}, OH, CONR₁₉R₂₀, SO₂NR₁₉R₂₀, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R_{11′}, OH, alkyl, haloalkyl, and aralkyl;
Y is selected from CH₂, C═N—OH, and CR₁₀R_{10′};
Rₐ and R_{b} are each independently selected from H and alkyl;
Z is selected from O, S, NR₁₇ and CR₁₈;
X is selected from O and NH;
p is 0, 1 or 2;
q is 0 or 1; and
r is 0 or 1;
wherein at least one of p, q and r is other than zero, and with the proviso that:
(i) when r is 0, p is 1 and q is 1, when X is NH, Z is other than 0;
(ii) when r and q are both 0, and p is 1, Z is not O;
R₁, R₄, and R₅ are each independently selected from H, alkyl, alkoxy, OH, F, C, Br, and I;
R₂ and R₃ are each independently selected from H, F, Cl, Br, I, CN, methoxy, haloalkyl, haloalkoxy and CO₂-alkyl;
R₁₀ and R_{10′} are each independently selected from H, F, alkyl, and haloalkyl;
R₁₁ and R_{11′} are each independently selected from H, alkyl, haloalkyl, COR₁₂, CO₂R₁₂ and SO₂R₁₃;
R₁₂ and R₁₃ are each independently alkyl;
R₁₅ and R₁₆ are each independently selected from H, alkoxy, alkyl and OH;
R₁₇ is selected from H, CN, OH, alkoxy and alkyl;
R₁₈ is NO₂ or CN;
each R₁₉ is independently H or alkyl; and
each R₂₀ is independently alkyl;
for use as a medicament.

44. A compound for use according to claim 43, for use in treating a disorder as defined in any one of claims 29 to 38 or claim 40.

45. A compound of formula (Ii), or a pharmaceutically acceptable salt or solvate thereof, 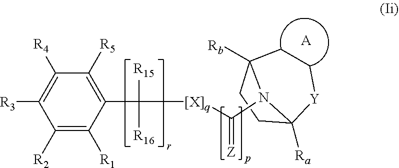
wherein:
ring A is a 5 or 6 membered aromatic or heteroaromatic ring, wherein said aromatic or heteroaromatic ring is optionally substituted with one or more substituents selected from halo, CN, alkoxy, NR₁₁R_{11′}, OH, CONR₁₉R₂₀, SO₂NR₁₉R₂₀, alkyl, haloalkyl, aralkyl, aryl, and heteroaryl, and wherein said aryl and heteroaryl substituents are in turn optionally substituted with one or more substituents each independently selected from halo, CN, alkoxy, NR₁₁R_{11′}, OH, alkyl, haloalkyl, and aralkyl;
Y is selected from CH₂, C═N—OH, and CR₁₀R_{10′};
Rₐ and R_{b} are each independently selected from H and alkyl;
Z is selected from O, S, NR₁₇ and CR₁₃;
X is selected from O and NH;
p is 0
q is 0; and
r is 1;
R₁, R₄, and R₅ are each independently selected from H, alkyl, alkoxy, OH, F, C, Br, and I;
R₂ is selected from H, F, C, Br, I, CN, methoxy, haloalkyl, haloalkoxy and CO₂-alkyl;
R₃ is selected from H, F, C, Br, I, CN, haloalkyl, haloalkoxy and CO₂-alkyl with the proviso that at least one of R₁-R₅ must be other than H;
R₁₀ and R_{10′} are each independently selected from H, F, alkyl, and haloalkyl;
R₁₁ and R_{11′} are each independently selected from H, alkyl, haloalkyl, COR₁₂, CO₂R₁₂ and SO₂R₁₃;
R₁₂ and R₁₃ are each independently alkyl;
R₁₅ and R₁₆ are each H;
R₁₇ is selected from H, CN, OH, alkoxy and alkyl;
R₁₈ is NO₂ or CN;
each R₁₉ is independently H or alkyl; and
each R₂₀ is independently alkyl.
